(19) United States
(12) Patent Application Publication
(10) Pub. No.: US 2012/0184548 A1
(43) Pub. Date: Jul. 19, 2012

(54) CARBOXYLIC ACID ARYL AMIDES

(76) Inventors: Romyr Dominique, Ridgewood, NJ (US); Kin-Chun Luk, North Caldwell, NJ (US); Qi Qiao, Bloomfield, NJ (US); Pamela Loreen Rossman, Nutley, NJ (US)

(21) Appl. No.: 13/347,726

(22) Filed: Jan. 11, 2012

Related U.S. Application Data
(60) Provisional application No. 61/434,001, filed on Jan. 19, 2011.

(51) Int. Cl. A61K 31/4704; C07D 413/12; A61P 25/28; A61P 35/00; A61P 43/00; C07D 215/54; A61K 31/5377

(52) U.S. Cl. 514/235.2; 546/156; 514/312; 544/128

(57) ABSTRACT

Compounds of formula

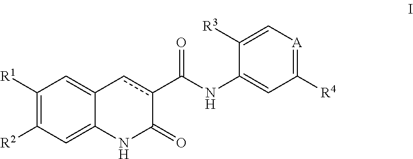

and pharmaceutically acceptable salts thereof are described, as well as the pharmaceutical compositions containing said compounds and their pharmaceutically acceptable salts, and the use of said compounds and pharmaceutical compositions for the treatment, control or amelioration of proliferative diseases, including cancer.

### PRIORITY TO RELATED APPLICATIONS

This application claims the benefit of U.S. Provisional Application No. 61/434,001, filed Jan. 19, 2011, which is hereby incorporated by reference in its entirety.

### FIELD OF THE INVENTION

The present invention relates to 2-oxo-1,2-dihydro-quinoline-3-carboxylic acid aryl amides which act as inhibitors of DYRK1B and/or DYRK1A and are useful in the amelioration, treatment or control of cancer, especially solid tumors, or in the amelioration, treatment or control of Down syndrome or Alzheimer's disease.

### BACKGROUND OF THE INVENTION

Kinases are known to be important cellular enzymes that regulate cellular functions such as regulating cell division and proliferation. WO 2008/047307. Dual-specificity tyrosine-phosphorylation-regulated kinases (DYRKs) are a subfamily of protein kinases that have dual-specificity and are believed to play roles in cell proliferation and apoptosis induction. See, e.g., Kiyotsugu Yoshida, “Role for DYRK family kinases on regulation of apoptosis,” Biochemical Pharmacology 76 (2008) pp 1389-1394; Jinghun Gao et al., “Mirk/Dyrk1B, a novel therapeutic target, mediates cells survival in non-small cell lung cancer cells,” Cancer Biology & Therapy 8:17 (2009) pp. 1671-1679. DYRK1A is believed to be implicated in neural differentiation. Yoshida, id. at 1390. Over expression of this kinase is believed to be involved in Down syndrome and Alzheimer's disease. See Nam Kim, “Putative therapeutic agents for learning and memory deficits of people with Down syndrome,” Bioorganic & Medicinal Chemistry Letters,” 16 (2006) pp 3772-76 and Joongkyu Park et al, “Function and regulation of Dyrk1A: towards understanding Down syndrome,” Cell. Mol. Life. Sci 66 (2009) pp 3235-3240. Thus, inhibition of this kinase is believed to be of benefit in controlling or ameliorating the effects of Down syndrome and early onset Alzheimer's disease. See, e.g., Kim, id; Park, id, and Kyung Koo et al., “QSAR analysis of pyrazolidine-3,5-diones derivatives as Dyrk1A inhibitors,” Bioorganic & Medicinal Chemistry Letters 19 (2009) pp 2324-2328.

DYRK1B (also referred to as MIRK) mediates survival and differentiation in many tissues. It is believed to be implicated in certain cancers, particularly solid tumors. See, e.g., Gao, supra (lung cancer cells); Kangmoon Lee et al, “Mirk Protein Kinase is a Mitogen-activated Protein Kinase Substrate that Mediates Survival of Colon Cancer Cells”, Cancer Research 60 (2000):3631-3637 and Xiaobing Deng et al, “The Kinase Mirk/Dyrk1B Mediates Cell Survival in a Pancreatic Ductal Adenocarcinoma,” Cancer Res 66:8 (2006) pp 4149-58 (pancreatic cancer cells). Thus, inhibition of this kinase is believed to be of benefit in controlling or ameliorating cancer. See, Cao Yang et al, “The kinase Mirk is a potential therapeutic target in osteosarcoma,” Carcinogenesis 31:4 (2010) pp 552-558 and Eileen Friedman, “The Kinase Mirk/dyrk1B: A Possible Therapeutic Target in Pancreatic Cancer,” Cancers 2 (2010) 1492-1512.

### SUMMARY OF THE INVENTION

One aspect of the invention is a compound of formula I

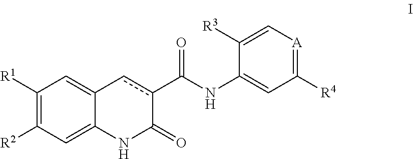

or a pharmaceutically acceptable salt thereof, wherein—, A, R¹, R², R³ and R⁴ are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or controlling cancer, preferably a solid tumor, in a mammal, preferably a human, comprising administering to said mammal a therapeutically effective amount of a compound according to the invention or a pharmaceutically acceptable salt thereof.

The present invention further relates to a method of treating, ameliorating or controlling Down syndrome or Alzheimer's disease in a human, comprising administering to said human a therapeutically effective amount of a compound according to the invention or a pharmaceutically acceptable salt thereof.

### DETAILED DESCRIPTION OF THE INVENTION

### Definitions

As used herein, the following terms shall have the following definitions.

The terms “C₁₋₆ alkyl” or “C₁₋₄ alkyl” refer to straight- or branched-chain saturated hydrocarbon groups having from 1 to 6, or 1 to 4, carbon atoms, respectively. Examples of C₁₋₆ alkyl groups include, but are not limited to, methyl, ethyl, n-propyl, i-propyl, n-butyl, s-butyl, t-butyl, n-pentyl, and s-pentyl.

“Alkoxy, alkoxyl or lower alkoxy” refers to any of the above alkyl groups which is attached to the remainder of the molecule by an oxygen atom (RO—). Typical alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Aryl” means a substituted or unsubstituted monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

The term “cycloalkyl” as used herein means any stable monocyclic or polycyclic system which consists of carbon atoms only, all rings of which are saturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds.

“Heteroaryl” means an aromatic heterocyclic ring system containing up to two rings, each of which independently can be substituted or unsubstituted. Preferred heteroaryl groups include, but are not limited to, thienyl (or thiophenyl), furyl, indolyl, pyrrolyl, pyridinyl, pyrazinyl, oxazolyl, thiaxolyl, quinolinyl, pyrimidinyl, imidazolyl, triazolyl and tetrazolyl.

In the case of a heteroaryl which is bicyclic it should be understood that one ring may be aryl while the other is heteroaryl and both being independently substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidynyl, including pyrrolidin-1-yl, pyrrolidin-2-yl and pyrrolidin-3-yl; piperazinyl; piperidinyl; morpholinyl, including morpholin-4-yl; and the like which in turn can be substituted.

In the case of a heterocycle that is bicyclic it should be understood that one ring may be heterocycle while the other is cycloalkyl, and both can be independently substituted or unsubstituted.

Hydroxy or hydroxyl is a prefix indicating the presence of a monovalent —O—H group.

“IC₅₀” refers to the concentration of a particular compound required to inhibit 50% of a specific measured activity. IC₅₀ can be measured, inter alia, as is described subsequently in Examples 75 and 76.

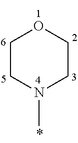

“Morpholin-4-yl” means the heterocycle residue.

The residue is attached to the remainder of the molecule at the 4 position designated by the asterisk.

“Oxazol-2-yl” means the residue

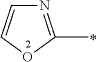

that is attached to the remainder of the molecule at the 2 position designated by the asterisk.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent.

“Tetrazol-5-yl” means the residue

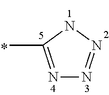

that is attached to the remainder of the molecule at the 5 position as designated by the asterisk.

In one embodiment, the present invention relates to compounds of formula I

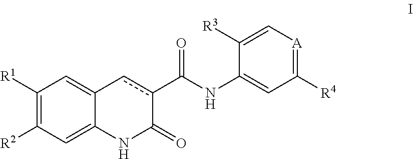

wherein [symbol]
means the presence of a bond (that is, a double bond is present in the above structure) or no bond (meaning a single bond is present);

### A is CH, CF or N;

R¹ is selected from the group consisting of
(a) OH,

(b) OR⁵, and

(c) NR⁶R⁷;

R² is selected from the group consisting of

(a) OR⁵, and

(b) NR⁶R⁷;

R³ is selected from the group consisting of

(a) C₁₋₄ alkyl,

(b) F,

(c) Cl, and

(d) Br;

R⁴ is selected from the group consisting of

(a) COR^{S},

(b) Tetrazol-5-yl, and

(c) Oxazol-2-yl,

and when A is N, R⁴ can also be H;

R⁵ is selected from the group consisting of

(a) C₁₋₄ alkyl,

(b) C₁₋₄ alkyl substituted with up to 3 groups selected from
heterocycle,
OH,
OC₁₋₄ alkyl, wherein the alkyl optionally may be substituted with one or more alkoxy groups,
NR⁹R¹⁰, and
CN;
   R⁶ and R⁷ are each independently selected from the group consisting of

(a) H,

(b) C₁₋₄ alkyl, and

(c) C₂₋₄ alkyl substituted with up to 3 groups selected from
heterocycle,
OH,
OC₁₋₄ alkyl,
NR⁹R¹⁰, and
CN;
   R⁸ is selected from the group consisting of

(a) OR¹¹, and

(b) NR¹²R¹³;

R⁹ and R¹⁹ are independently selected from the group consisting of

(a) H, and

(b) C₁₋₄ alkyl;

R¹¹ is selected from the group consisting of

(a) C₁₋₆ alkyl,

(b) C₁₋₆ alkyl substituted with up to 3 groups selected from
aryl,
aryl optionally substituted with Cl, F, CH₃,
heteroaryl,
cycloalkyl,
heterocycle,
OH,
OC₁₋₄ alkyl,
NR⁹R¹⁰,
CN, and
CONR⁹R¹⁰, and

(c) aryl optionally substituted with Cl, F, CH₃;

R¹² and R¹³ are independently selected from the group consisting of

(a) H,

(b) C₁₋₆ alkyl,

(c) C₁₋₆ alkyl substituted with up to 3 groups selected from
aryl,
aryl optionally substituted with Cl, F, CH₃,
heteroaryl,
cycloalkyl,
heterocycle,
OH,
OC₁₋₄ alkyl,
NR⁹R¹⁰,
CN,
CONR⁹R¹⁰, and

(d) Aryl optionally substituted with Cl, F, CH₃,

or, alternately, NR¹²R¹³ together can form a heterocycle, optionally substituted with
(a) Cl,
(b) F,
(c) CH₃,
(d) aryl optionally substituted with Cl, F, CH₃, and
(e) heteroaryl optionally substituted with Cl, F, CH₃;

or a pharmaceutically acceptable salt thereof.

In another embodiment, the invention relates to compounds of formula I wherein—in the heterocyclic ring is a bond thus indicating the presence of a double bond, namely a compound of formula

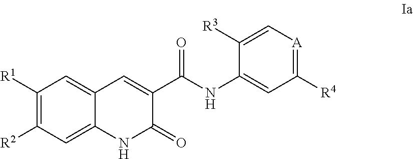

wherein A, R¹, R², R³ and R⁴ are as defined above, or a pharmaceutically acceptable salt thereof.

In another embodiment, the invention relates to compounds of formula I wherein—in the heterocyclic ring is not a bond thus indicating the presence of a single bond, namely a compound of formula

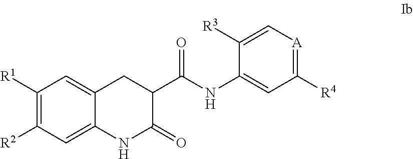

wherein A, R¹, R², R³ and R⁴ are as defined above, or a pharmaceutically acceptable salt thereof.

Another embodiment of the invention relates to compounds of Formula I, or a pharmaceutically acceptable salt thereof, wherein A is CH.

Another embodiment of the invention relates to compounds of Formula I, or a pharmaceutically acceptable salt thereof, wherein A is N.

Another embodiment of the invention relates to compounds of Formula I, or a pharmaceutically acceptable salt thereof, wherein R¹ is OR⁵, and R⁵ is C₁₋₄ alkyl or C₁₋₄ alkyl substituted by heterocycle, OC₁₋₄ alkyl or NR⁹R¹⁰.

Another embodiment of the invention relates to compounds of Formula I, or a pharmaceutically acceptable salt thereof, wherein R¹ is OR⁵ and R⁵ selected from CH₃, CH₂CH₂OCH₃ and CH₂CH₂-heterocycle.

Another embodiment of the invention relates to compounds of Formula I, or a pharmaceutically acceptable salt thereof, wherein R² is OR⁵ and R⁵ is C₁₋₄ alkyl.

Another embodiment of the invention relates to compounds of Formula I, or a pharmaceutically acceptable salt thereof, wherein R² is OCH₃.

Another embodiment of the invention relates to compounds of Formula I, or a pharmaceutically acceptable salt thereof, wherein R³ is C₁₋₄ alkyl or Cl.

Another embodiment of the invention relates to compounds of Formula I, or a pharmaceutically acceptable salt thereof, wherein R³ is CH₃.

Another embodiment of the invention relates to compounds of Formula I, or a pharmaceutically acceptable salt thereof, wherein R⁴ is COR⁸, and R⁸ is OR¹¹.

Another embodiment of the invention relates to compounds of Formula I, or a pharmaceutically acceptable salt thereof, wherein R⁴ is COR⁸, and R⁸ is OR¹¹ and R¹¹ is C₁₋₆ alkyl. In another embodiment, R¹¹ is CH₃.

Another embodiment of the invention relates to compounds of Formula I, or a pharmaceutically acceptable salt thereof, wherein R⁴ is COR⁸ and R⁸ is NR¹²R¹³.

In another embodiment, R⁴ is COR⁸, R⁸ is NR¹²R¹³ and R¹² and R¹³ are independently selected from H and C₁₋₆ alkyl, or a pharmaceutically acceptable salt thereof. In an embodiment R¹² is CH₃, or a pharmaceutically acceptable salt thereof. In another embodiment R¹³ is CH₃, or a pharmaceutically acceptable salt thereof.

In another embodiment, R⁴ is COR⁸, R⁸ is NR¹²R¹³ and R¹² and R¹³ are independently selected from H, C₁₋₆ alkyl, and C₁₋₆ alkyl that is substituted by up to 3 groups selected from aryl, OH, C₁₋₄ alkyl, heterocycle, cycloalkyl, and NR⁹R¹⁰, optionally NH₂, or a pharmaceutically acceptable salt thereof. In an embodiment NR¹²R¹³ is NH₂. In another embodiment R¹² and R¹³ are each CH₃. In another embodiment, one of R¹² or R¹³ is H and the other is C₁₋₆ alkyl substituted with cyclohexyl, heterocycle, OH, C₁₋₄ alkyl, NH₂ or phenyl that is optionally substituted with Cl.

In another embodiment, R⁴ is COR⁸, R⁸ is NR¹²R¹³ and R¹² and R¹³ are independently selected from H and C₁₋₆ alkyl that is substituted with up to 3 groups selected from aryl, heteroaryl, OH, lower alkyl, heterocycle, cycloalkyl and NR⁹R¹⁰, or a pharmaceutically acceptable salt thereof.

In another embodiment, R⁹ and R¹⁰ are independently selected from H

Another embodiment of the invention relates to compounds of Formula I, or a pharmaceutically acceptable salt thereof, wherein A is N and R⁴ is H.

Another embodiment of the invention relates to compounds of Formula I, or a pharmaceutically acceptable salt thereof, wherein R⁴ is tetrazol-5-yl or oxazol-2-yl.

Another embodiment of the invention relates to compounds of Formula

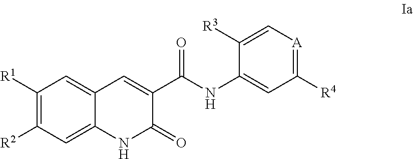

wherein

### A is CH or N

R¹ is selected from the group consisting of OH, OCH₃, OCH₂CH₂OCH₃, OCH₂CH₂-4-morpholinyl and O(CH₂)₂O(CH₂)₂OCH₃;
R² is selected from the group consisting of OCH₃, OCH₂CH₂OCH₃;
R³ is selected from the group consisting of CH₃ and Cl;
R⁴ is selected from the group consisting of C(O)OCH₃, C(O)N(CH₃)₂, tetrazol-5-yl and COR⁸;

### R⁸ is NR¹²R¹³; and

R¹² and R¹³ are each independently selected from

H, and

C₁₋₆ alkyl substituted with NH₂, OH, morpholin-4-yl, cyclohexyl and phenyl optionally substituted with Cl;

or a pharmaceutically acceptable salt thereof.

Another embodiment is a compound of formula Ib wherein

A is CH;

R¹ and R² are OCH₃;

R³ is C₁ or CH₃; and

R⁴ is C(O)OCH₃ or C(O)NHCH₂-phenyl;

or a pharmaceutically acceptable salt thereof.

It is contemplated herein that salts of compounds of formula I such as hydrochloride or trifluoroacetic acid salts include salts with multiple conjugates such as mono HCl, di-HCl, etc.

Compounds according to the invention include:
3-[(6,7-Dimethoxy-2-oxo-1,2-dihydro-quinoline-3-carbonyl)-amino]-4-methyl-benzoic acid methyl ester (Example 17);
4-Chloro-3-[(6,7-dimethoxy-2-oxo-1,2-dihydro-quinoline-3-carbonyl)-amino]-benzoic acid methyl ester (Example 19);
6,7-Dimethoxy-2-oxo-1,2-dihydro-quinoline-3-carboxylic acid (2-chloro-5-dimethylcarbamoyl-phenyl)-amide (Example 20);
6,7-Dimethoxy-2-oxo-1,2-dihydro-quinoline-3-carboxylic acid (3-methyl-pyridin-4-yl)-amide (Example 21);
6,7-Dimethoxy-2-oxo-1,2-dihydro-quinoline-3-carboxylic acid [2-methyl-5-(1H-tetrazol-5-yl)-phenyl]-amide (Example 22);
3-{[7-Methoxy-6-(2-methoxy-ethoxy)-2-oxo-1,2-dihydro-quinoline-3-carbonyl]-amino}-4-methyl-benzoic acid methyl ester (Example 23);
7-Methoxy-6-(2-methoxy-ethoxy)-2-oxo-1,2-dihydro-quinoline-3-carboxylic acid [2-methyl-5-(1H-tetrazol-5-yl)-phenyl]-amide (Example 24);
3-{[6,7-Bis-(2-methoxy-ethoxy)-2-oxo-1,2-dihydro-quinoline-3-carbonyl]-amino}-4-methyl-benzoic acid methyl ester (Example 25);
3-{[6-Methoxy-7-(2-methoxy-ethoxy)-2-oxo-1,2-dihydro-quinoline-3-carbonyl]-amino}-4-methyl-benzoic acid methyl ester (Example 26);
6-Methoxy-7-(2-methoxy-ethoxy)-2-oxo-1,2-dihydro-quinoline-3-carboxylic acid [2-methyl-5-(1H-tetrazol-5-yl)-phenyl]-amide (Example 27);
6,7-Bis-(2-methoxy-ethoxy)-2-oxo-1,2-dihydro-quinoline-3-carboxylic acid [2-methyl-5-(1-tetrazol-5-yl)-phenyl]-amide (Example 28);
6,7-Dimethoxy-2-oxo-1,2-dihydro-quinoline-3-carboxylic acid [5-(2-amino-1-phenyl-ethylcarbamoyl)-2-methyl-phenyl]-amide (Example 31);
6,7-Dimethoxy-2-oxo-1,2-dihydro-quinoline-3-carboxylic acid [2-methyl-5-(3-morpholin-4-yl-propylcarbamoyl)-phenyl]-amide (Example 32);
6,7-Dimethoxy-2-oxo-1,2-dihydro-quinoline-3-carboxylic acid [5-(1-hydroxymethyl-2-methyl-propylcarbamoyl)-2-methyl-phenyl]-amide (Example 33);
6,7-Dimethoxy-2-oxo-1,2-dihydro-quinoline-3-carboxylic acid [5-(3-amino-1-phenyl-propylcarbamoyl)-2-methyl-phenyl]-amide (Example 35);
6,7-Dimethoxy-2-oxo-1,2-dihydro-quinoline-3-carboxylic acid (3-chloro-pyridin-4-yl)-amide (Example 36);
7-Methoxy-6-(2-methoxy-ethoxy)-2-oxo-1,2-dihydro-quinoline-3-carboxylic acid [5-(2-amino-1-phenyl-ethylcarbamoyl)-2-methyl-phenyl]-amide (Example 37);
7-Methoxy-6-(2-methoxy-ethoxy)-2-oxo-1,2-dihydro-quinoline-3-carboxylic acid [5-(3-amino-1-phenyl-propylcarbamoyl)-2-methyl-phenyl]-amide (Example 38);
3-{[7-Methoxy-6-(2-morpholin-4-yl-ethoxy)-2-oxo-1,2-dihydro-quinoline-3-carbonyl]-amino}-4-methyl-benzoic acid methyl ester (Example 39);
6,7-Dimethoxy-2-oxo-1,2-dihydro-quinoline-3-carboxylic acid [5-(3-chloro-benzylcarbamoyl)-2-methyl-phenyl]-amide (Example 40);
6,7-Dimethoxy-2-oxo-1,2-dihydro-quinoline-3-carboxylic acid [5-(cyclohexylmethyl-carbamoyl)-2-methyl-phenyl]-amide (Example 41);
6,7-Dimethoxy-2-oxo-1,2-dihydro-quinoline-3-carboxylic acid (5-benzylcarbamoyl-2-methyl-phenyl)-amide (Example 42);
7-Methoxy-6-(2-morpholin-4-yl-ethoxy)-2-oxo-1,2-dihydro-quinoline-3-carboxylic acid
[5-(3-chloro-benzylcarbamoyl)-2-methyl-phenyl]-amide (Example 43);
7-Methoxy-6-(2-morpholin-4-yl-ethoxy)-2-oxo-1,2-dihydro-quinoline-3-carboxylic acid
[5-(3-amino-1-phenyl-propylcarbamoyl)-2-methyl-phenyl]-amide (Example 44);
6,7-Dimethoxy-2-oxo-1,2-dihydro-quinoline-3-carboxylic acid [5-((R)-3-amino-1-phenyl-propylcarbamoyl)-2-methyl-phenyl]-amide (Example 47);
6,7-Dimethoxy-2-oxo-1,2-dihydro-quinoline-3-carboxylic acid [5-((S)-3-amino-1-phenyl-propylcarbamoyl)-2-methyl-phenyl]-amide (Example 48);
6,7-Dimethoxy-2-oxo-1,2-dihydro-quinoline-3-carboxylic acid [5-[2-amino-1-(3-chloro-phenyl)-ethylcarbamoyl]-2-methyl-phenyl]-amide (Example 50);
6,7-Dimethoxy-2-oxo-1,2-dihydro-quinoline-3-carboxylic acid (2-methyl-5-oxazol-2-yl-phenyl)-amide (Example 51);
7-Methoxy-6-(2-methoxy-ethoxy)-2-oxo-1,2-dihydro-quinoline-3-carboxylic acid [5-(3-chloro-benzylcarbamoyl)-2-methyl-phenyl]-amide (Example 52);
Methyl 4-chloro-3-(7-methoxy-6-(2-methoxyethoxy)-2-oxo-1,2-dihydroquinoline-3-carboxamido)benzoate (Example 53);
N-(2-Chloro-5-(3-chlorobenzylcarbamoyl)phenyl)-7-methoxy-6-(2-methoxyethoxy)-2-oxo-1,2-dihydroquinoline-3-carboxamide (Example 55);
(R)-N-(5-(2-Hydroxy-1-phenylethylcarbamoyl)-2-methylphenyl)-6,7-dimethoxy-2-oxo-1,2-dihydroquinoline-3-carboxamide (Example 56);
(S)-N-(5-(2-Hydroxy-1-phenylethylcarbamoyl)-2-methylphenyl)-6,7-dimethoxy-2-oxo-1,2-dihydroquinoline-3-carboxamide (Example 57);
7-Methoxy-6-(2-methoxy-ethoxy)-2-oxo-1,2-dihydro-quinoline-3-carboxylic acid [5-((R)-3-amino-1-phenyl-propylcarbamoyl)-2-methyl-phenyl]-amide trifluoro-acetic acid salt (Example 58);
7-Methoxy-6-(2-methoxy-ethoxy)-2-oxo-1,2-dihydro-quinoline-3-carboxylic acid [5-((S)-3-amino-1-phenyl-propylcarbamoyl)-2-methyl-phenyl]-amide trifluoro-acetic acid salt (Example 59);
N-(5-(2-Amino-1-(3-chlorophenyl)ethylcarbamoyl)-2-chlorophenyl)-7-methoxy-6-(2-methoxyethoxy)-2-oxo-1,2-dihydroquinoline-3-carboxamide (Example 60);
N-(5-(3-Amino-1-phenylpropylcarbamoyl)-2-chlorophenyl)-7-methoxy-6-(2-methoxyethoxy)-2-oxo-1,2-dihydroquinoline-3-carboxamide (Example 61);
N-(5-(2-Amino-1-phenylethylcarbamoyl)-2-chlorophenyl)-7-methoxy-6-(2-methoxyethoxy)-2-oxo-1,2-dihydroquinoline-3-carboxamide (Example 62);
(R)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide (Example 63);
(S)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide (Example 64);
(S)-N-(5-(1-Hydroxy-4-methylpentan-2-ylcarbamoyl)-2-methylphenyl)-6,7-dimethoxy-2-oxo-1,2-dihydroquinoline-3-carboxamide (Example 65);
(R)-N-(5-(1-Hydroxy-4-methylpentan-2-ylcarbamoyl)-2-methylphenyl)-6,7-dimethoxy-2-oxo-1,2-dihydroquinoline-3-carboxamide (Example 66);
6-Hydroxy-7-methoxy-2-oxo-1,2-dihydro-quinoline-3-carboxylic acid [5-(3-chloro-benzylcarbamoyl)-2-methyl-phenyl]-amide (Example 68);
7-Methoxy-6-[2-(2-methoxy-ethoxy)-ethoxy]-2-oxo-1,2-dihydro-quinoline-3-carboxylic acid [5-(3-chloro-benzylcarbamoyl)-2-methyl-phenyl]amide (Example 70);
4-Chloro-3-[(6,7-dimethoxy-2-oxo-1,2,3,4-tetrahydro-quinoline-3-carbonyl)-amino]-benzoic acid methyl ester (Example 71);
3-[(6,7-Dimethoxy-2-oxo-1,2,3,4-tetrahydro-quinoline-3-carbonyl)-amino]-4-methyl-benzoic acid methyl ester (Example 72);
6,7-Dimethoxy-2-oxo-1,2,3,4-tetrahydro-quinoline-3-carboxylic acid (5-benzylcarbamoyl-2-chloro-phenyl)-amide (Example 73); and
6,7-Dimethoxy-2-oxo-1,2,3,4-tetrahydro-quinoline-3-carboxylic acid (5-benzylcarbamoyl-2-methyl-phenyl)-amide (Example 74);
   and the pharmaceutically acceptable salts of the foregoing compounds.

The compounds of formula I, including compounds of formula Ia and Ib, as well as their salts that have at least one asymmetric carbon atom may be present as racemic mixtures or different stereoisomers. The various isomers can be isolated by known separation methods, e.g., chromatography. In one embodiment, the invention relates to compounds of formula I having the S configuration. In another embodiment, the invention relates to compounds of formula I having the R configuration. Depending on where the chiral centers are for each molecule, for some molecules of the invention the R configuration may be preferred while for others the S configuration may be preferred.

Compounds disclosed herein and covered by formula I, including compounds of formula Ia and Ib, above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention that are inhibitors of DYRK1B are useful in the treatment, amelioration or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, breast, colon, lung and prostate tumors.

Compounds that are inhibitors of DYRK1A are useful in the treatment, amelioration or control of Down Syndrome and Alzheimer's Disease.

A “therapeutically effective amount” or “effective amount” of a compound in accordance with this invention means an amount of compound that is effective to alleviate, ameliorate or control symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof, and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

### General Synthesis of the Compounds according to the Invention

The present invention provides methods for the synthesis of the 2-oxo-1,2-dihydro-quinoline-3-carboxilic acid amides of the invention.

The compounds of the invention can be prepared by processes known in the art. Suitable processes for synthesizing these compounds are also provided in the examples. Generally, compounds of formula I can be synthesized according to one of the below described synthetic routes.

The key transformations are coupling reactions of carbonyl and carboxy starting materials and intermediates.

The starting materials are either commercially available or can be synthesized by methods known to those of ordinary skill in the art. Preparations of intermediates 3, 5, and 6 are illustrated in Schemes 1-4 below. In general, a suitable amino-aldehyde or ketone can be reacted with dialkyl malonate and base to generate the bicyclic ester 2 as a crude product, which can be hydrolyzed to the corresponding carboxylic acid 3. A standard amide coupling reaction between 3 and 4 can provide compounds of the invention (see Schemes 1-4 below). Alternately, if R″ in intermediate 4 is an ester (Scheme 2), then the ester in intermediate 5 can further be modified by hydrolysis to the corresponding acid 6 (Scheme 3). Acid 6 can then be converted to additional compounds of this invention via another esterification or amide formation (Scheme 4).

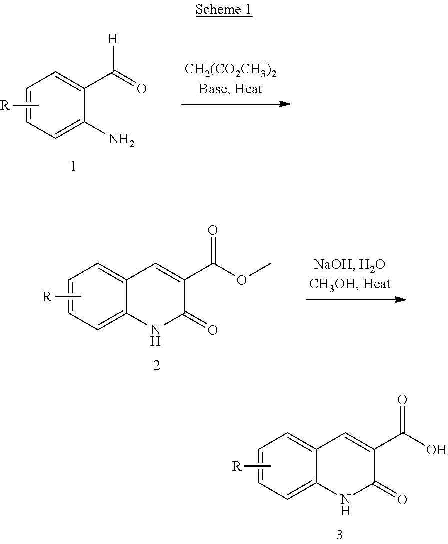

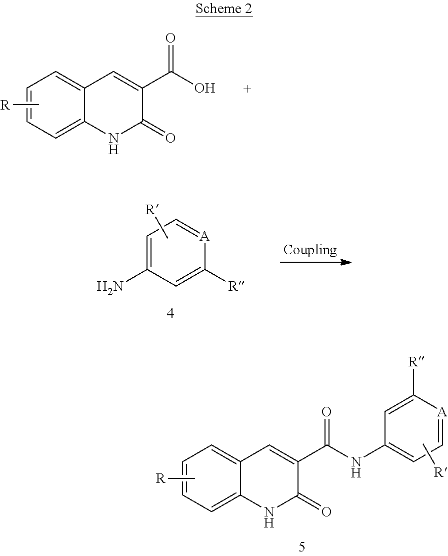

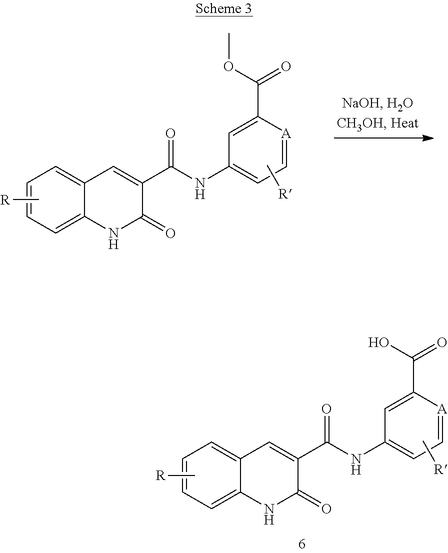

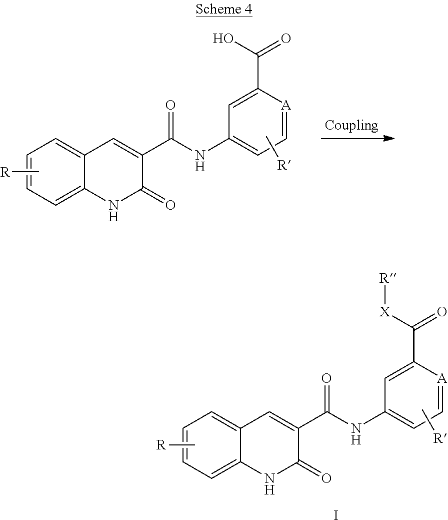

The synthesis of various phenyl components forming the right-hand half of compounds of formula I wherein A is CH, CF or N are shown in Schemes 5-7 below. These starting materials (e.g. compound 8) can be used in Scheme 2 above in place of compound 4 to yield the compound of formula I.

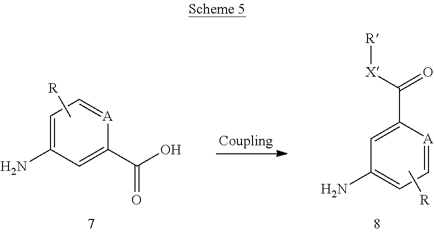

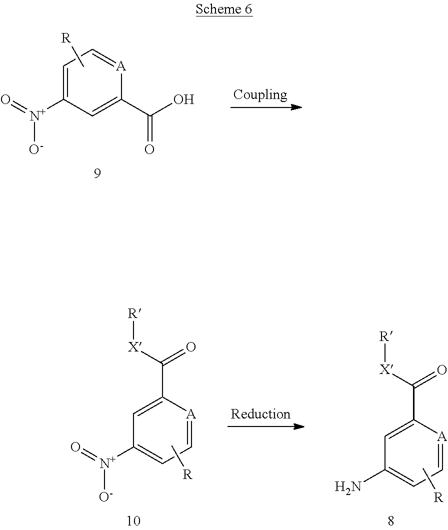

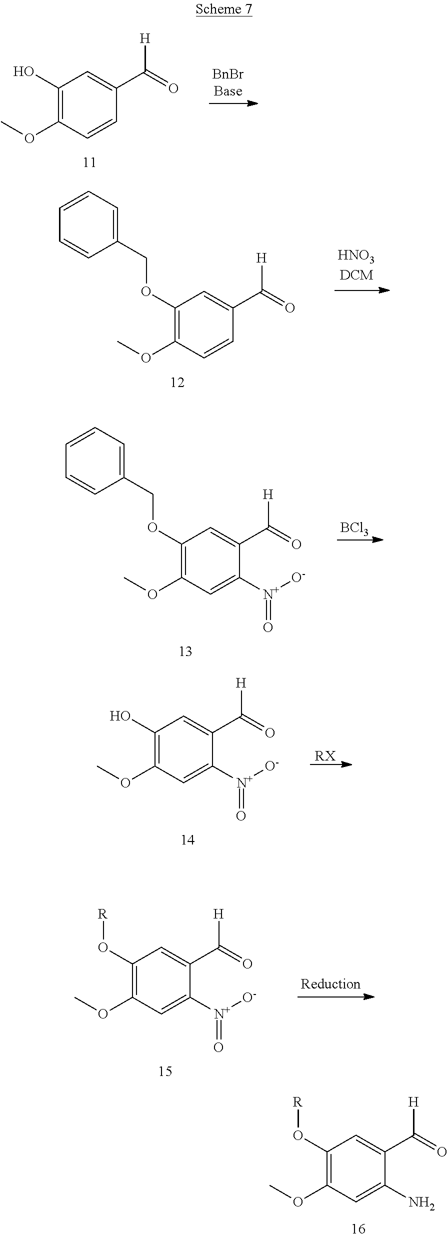

The synthesis of compounds of formula Ib can be achieved by modifying the first step of the synthetic scheme (see Scheme 8). In general, a suitable amino-aldehyde or ketone can be condensed with dialkyl malonate, acetic anhydride and base to generate 17. The double bond in compound 17 can then be reduced and the resulting amino-diester 18 can be cyclized to form the bicyclic ester 19. Compound 19 can be converted to compounds Ib of this invention following similar procedures to the synthesis of compounds 1a supra.

In case of certain amines that contain additional functional groups, appropriate protecting groups (for example tert-butoxy-carbonyl group) may be employed to facilitate synthesis. If such protecting groups are employed, the removal of such protecting groups to generate the compounds of the invention can be accomplished by standard methods known to those skilled in the art of organic synthesis.

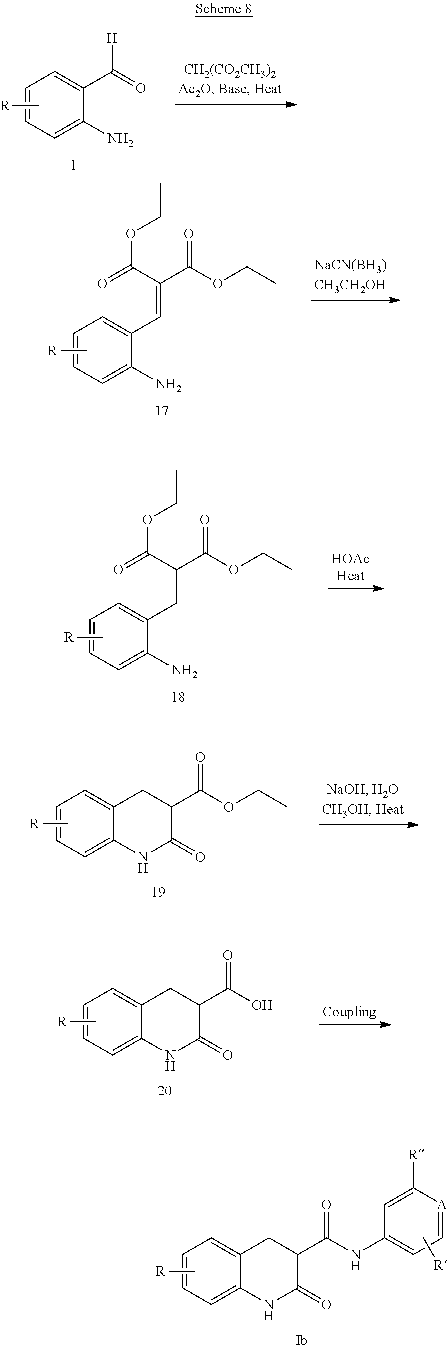

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using AutoNom 2000 Add-in v4.0 SP2 (function in ISIS Draw, Elsevier/MDL), or AutoNom 2000 TT v4.01.305 (Elsevier/MDL), or functions available in ChemDraw Pro Control 11.0.2 (CambridgeSoft Corp.).

### Abbreviations Used in the Examples

HRMS High Resolution Mass Spectrometry
LC-MS Liquid Chromatography Mass Spectrometry
RT (or rt) Room temperature
Min Minutes
H Hours
Ac₂O acetic anhydride
Boc₂O di-tert-butyl dicarbonate
Bu₄NI tetrabutyl ammonium iodide
BuOH butanol
tBuONa sodium t-butoxide
CDCl₃ chloroform-d
CD₃OD methanol-d₄
CF₃CO₂H trifluoroacetic acid
CH₂Cl₂ dichloromethane
CH₃ CN acetonitrile
CH₂(CO₂CH₃)₂ dimethyl malonate
C₂H₅OH ethanol
m-CPBA meta-chloroperbenzoic acid
Dave-PHOS 2-(2-dicyclohexylphosphanylphenyl)-N,N-dimethylaniline
DCM dichloromethane
DEAD diethyl azodicarboxylate
DIPEA N,N-diisopropylethylamine
DMF N,N-dimethylformamide
DMSO dimethylsulfoxide
D₂O deuterium oxide
Et₃N triethylamine
EtOAc ethyl acetate
(EtO)₃CH triethyl orthoformate
EtOH ethanol
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HCl hydrogen chloride
HCO₂NH₄ ammonium formate
H₂O water
HOAc acetic acid
HPLC high performance liquid chromatography
H₂SO₄ sulfuric acid
IPA 2-propanol
LAH lithium aluminum hydride
LiAlH₄ lithium aluminum hydride
LiOH lithium hydroxide
KCN potassium cyanide
K₂CO₃ potassium carbonate
K₃PO₄ potassium phosphate
MeCN acetonitrile
MeOH methanol
MgSO₄ magnesium sulfate
MnO₂ manganese dioxide
NaClO sodium hypochlorite
NaH sodium hydride
NaHCO₃ sodium bicarbonate
NaOH sodium hydroxide
Na₂SO₄ sodium sulfate
NH₃ ammonia
NH₄Cl ammonium chloride
Pd₂(dba)₃ tris(dibenzylideneacetone)dipalladium(0)
Pd(OAc)₂ palladium(II)acetate
POCl₃ phosphorous oxychloride
i-PrMgCl isopropyl magnesium chloride
PPh₃ triphenylphosphine
SEM-C1 2-(trimethylsilyl)ethoxymethyl chloride
TBAB tetrabutyl ammonium bromide
TEA triethylamine
TEMPO 2,2,6,6-tetramethylpiperidine 1-oxyl
TFA trifluoroacetic acid
THF tetrahydrofuran
TLC thin layer chromatography

### Example 1

### (2-Amino-2-phenyl-ethyl)-carbamic acid tert-butyl ester

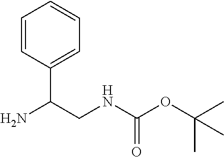

(2-Amino-2-phenyl-ethyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### (2-Hydroxy-2-phenyl-ethyl)-carbamic acid tert-butyl ester

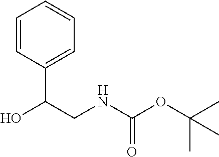

To a stirred solution of 2-amino-1-phenylethanol (20 g, 145.8 mmol) in THF (300 mL) was added the solution of Boc₂O (31.1 g, 153.1 mmol) in THF (100 mL) at 0° C. After addition, the mixture was stirred at room temperature for 0.5 hour. This mixture was concentrated to give the pure (2-hydroxy-2-phenyl-ethyl)-carbamic acid tert-butyl ester as a white solid. (Yield 34.4 g, 100%).

### Step B

### [2-(1,3-Dioxo-1,3-dihydro-isoindol-2-yl)-2-phenyl-ethyl]-carbamic acid tert-butyl ester

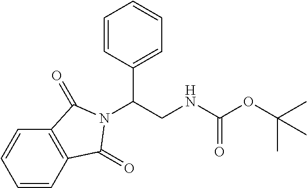

To a solution of (2-hydroxy-2-phenyl-ethyl)-carbamic acid tert-butyl ester (34.4 g, 145.0 mmol), phthalimide (21.3 g, 145 mmol), and PPh₃ (49.4 g, 188.5 mmol) was added drop-wise DEAD (32.8 g, 188.5 mmol) under stirring at 0° C. After addition, the mixture was stirred at room temperature for an additional 1 hour. The mixture was concentrated under reduced pressure. The residue was purified by column chromatography on silica gel (petroleum ether:ethyl acetate, 20:1 to 5:1) to give [2-(1,3-dioxo-1,3-dihydro-isoindol-2-yl)-2-phenyl-ethyl]-carbamic acid tert-butyl ester as a white solid. (Yield 39 g, 74%)

¹H NMR (300 MHz, CDCl₃): δ 7.88-7.80 (m, 2H), 7.74-7.68 (m, 2H), 7.49-7.47 (m, 2H), 7.38-7.26 (m, 3H), 5.56-5.50 (m, 1H), 4.83 (brs, 1H), 4.28-4.22 (m, 1H), 3.93-3.87 (m, 1H), 1.35 (s, 9H). LC-MS: [M-Boc+H]⁺267.

### Step C

### (2-Amino-2-phenyl-ethyl)-carbamic acid tert-butyl ester

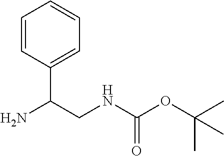

To a solution of [2-(1,3-dioxo-1,3-dihydro-isoindol-2-yl)-2-phenyl-ethyl]-carbamic acid tert-butyl ester (23 g, 63 mmol) in THF (180 mL) and MeOH (180 mL) was added 85% hydrazine hydrate (37 mL, 630 mmol) slowly. The resulting mixture was heated at 65° C. for 15 hours. The reaction mixture was cooled to room temperature, then concentrated to dryness. The residue was purified by column chromatography on silica gel (dichloromethane:MeOH, 100:1, 1% NH₃ H₂O) to give (2-amino-2-phenyl-ethyl)-carbamic acid tert-butyl ester as a white solid. (Yield 7.4 g, 50%).

¹H NMR (300 MHz, CDCl₃): δ 7.35-7.24 (m, 5H), 4.81 (brs, 1H), 4.08-4.03 (m, 1H), 3.38-3.21 (m, 2H), 1.44 (s, 9H). LC-MS: [M+H]⁺237.

### Example 2

### ((S)-2-Amino-2-phenyl-ethyl)-carbamic acid tert-butyl ester

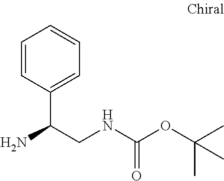

### Step A

### [(R)-2-Phenyl-propyl]-carbamic acid tert-butyl ester

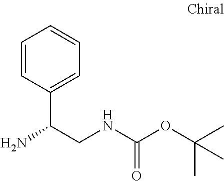

To a solution of R-(−)-2-amino-1-phenylethanol (6.52 g, 47.53 mmol) (Lancaster) in tetrahydrofuran (50 mL) was added di-tert-butyl dicarbonate (11.41 g, 52.78 mmol) (Aldrich) at room temperature with magnetic stirring. After 3 hours, mixture was concentrated and the residue was purified by flash chromatography eluting with 0-40% ethyl acetate in hexanes to give [(R)-2-phenyl-propyl]-carbamic acid tert-butyl ester. (Yield 10.05 g, 89.1%).

### Step B

### [(S)-2-(1,3-Dioxo-indan-2-yl)-2-phenyl-ethyl]carbamic acid tert-butyl ester

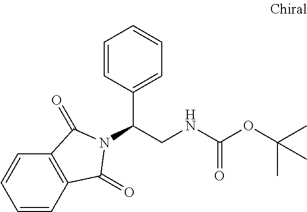

To a solution of [(R)-2-phenyl-propyl]-carbamic acid tert-butyl ester (10.05 g, 42.35 mmol), phthalimide (6.85 g, 46.59 mmol) (Aldrich) and triphenylphosphine (14.44 g, 55.06 mmol) (Aldrich) in tetrahydrofuran (150 mL) was added diisopropylazodicarboxylate (10.84 mL, 55.06 mmol) (Aldrich) dropwise at room temperature with magnetic stirring. After 18 hours, mixture was concentrated and the residue was purified by flash chromatography eluting with 15-30% ethyl acetate in hexanes to give [(S)-2-(1,3-dioxo-indan-2-yl)-2-phenyl-ethyl]carbamic acid tert-butyl ester. (Yield 15.04 g, 96.9%).

### Step C

### ((S)-2-Amino-2-phenyl-ethyl)-carbamic acid tert-butyl ester

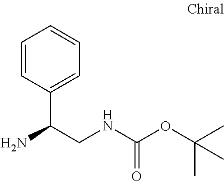

A solution of [(S)-2-(1,3-dioxo-indan-2-yl)-2-phenyl-ethyl]carbamic acid tert-butyl ester (5.9 g, 16.1 mmol) and anhydrous hydrazine (5.1 mL, 0.16 mol) (Aldrich) in methanol (20 mL) was heated at 60° C. for 18 hours. Solid was filtered off and washed with methanol. The solution was concentrate. The residue was diluted with dichloromethane and 1N NaOH solution. The aqueous phase was extracted with dichloromethane (1×). The combined organic phase was washed with brine, dried (MgSO₄) and concentrated. The residue was purified by flash chromatography eluting with 0-10% methanol in dichloromethane to give ((S)-2-amino-2-phenyl-ethyl)-carbamic acid tert-butyl ester. (Yield 1.84 g, 48.4%).

### Example 3

### (3-Amino-3-phenyl-propyl)-carbamic acid tert-butyl ester

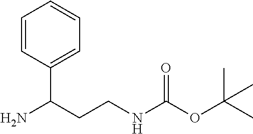

(3-Amino-3-phenyl-propyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-phenyl-propan-1-ol

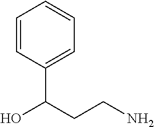

To a stirred suspension of LAH (20 g, 517 mmol) in dry THF (500 mL) was added a solution of 3-oxo-3-phenylpropanenitrile (30 g, 207 mmol) in dry THF (300 mL) drop-wise at 0° C. under nitrogen atmosphere. The mixture was warmed to 25° C. and then heated at 70° C. for 2 hours. After cooling to 0° C., a saturated solution of sodium hydroxide was added drop-wise and extracted with dichloromethane (200 mL). The organic solution was dried over anhydrous sodium sulfate and concentrated to dryness. The residue was purified by column chromatography (methanol:dichloromethane, 1:10) to afford 3-amino-1-phenyl-propan-1-ol. (Yield 30 g, crude). LC-MS: [M+H]⁺152.

### Step B

### (3-Hydroxy-3-phenyl-propyl)-carbamic acid tert-butyl ester

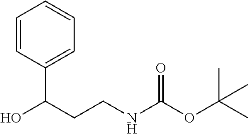

Et₃N (1.36 g, 14 mmol) was added to a solution of 3-amino-1-phenyl-propan-1-ol (1.7 g, 11.3 mmol) in THF (20 mL) under stirring. Boc₂O (3.0 g, 13.7 mmol) in THF (20 mL) was added dropwise to the solution at 0° C. Then the resulting mixture was warmed to room temperature and stirred for an additional 2 hours. The mixture was concentrated under reduced pressure. The residue was purified by column chromatography on silica gel (petroleum ether:ethyl acetate, 3:1) to give (3-hydroxy-3-phenyl-propyl)-carbamic acid tert-butyl ester. (Yield 1.7 g, 60%). LC-MS: [M+Na]⁺274.

### Step C

### [3-(1,3-Dioxo-1,3-dihydro-isoindol-2-yl)-3-phenyl-propyl]-carbamic acid tert-butyl ester

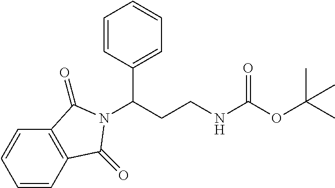

To a solution of (3-hydroxy-3-phenyl-propyl)-carbamic acid tert-butyl ester (10.4 g, 41.4 mmol), phthalimide (5.2 g, 36.6 mmol), and PPh₃ (14.6 g, 55.5 mmol) in THF (204 mL) was added dropwise DEAD (8.9 mL, 55 mmol) with stirring at 0° C. Then the resulting mixture was warmed to room temperature for an additional 2 hours. The mixture was concentrated under reduced pressure. The residue was purified by column chromatography on silica gel (petroleum ether:ethyl acetate, 3:1) to give [3-(1,3-dioxo-1,3-dihydro-isoindol-2-yl)-3-phenyl-propyl]-carbamic acid tert-butyl ester. (Yield 10.5 g, 66.8%).

¹H NMR (300 MHz, CDCl₃): δ 7.81-7.75 (m, 2H), 7.69-7.64 (m, 2H), 7.53-7.50 (m, 2H), 7.34-7.23 (m, 3H), 5.44-5.38 (m, 1H), 4.74 (brs, 1H), 3.29-3.07 (m, 2H), 2.83-2.75 (m, 1H), 2.51-2.42 (m, 1H), 1.42 (s, 9H). LC-MS: [M-Boc+H]⁺281.

### Step D

### (3-Amino-3-phenyl-propyl)-carbamic acid tert-butyl ester

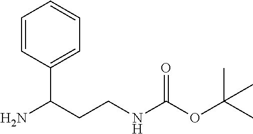

85% Hydrazine hydrate (5.1 mL, 74 mmol) was added to a solution of [3-(1,3-dioxo-1,3-dihydro-isoindol-2-yl)-3-phenyl-propyl]-carbamic acid tert-butyl ester (2.8 g, 7.4 mmol) in THF (25 mL) and MeOH (25 mL). The resulting mixture was heated at 65° C. for 6 hours. Then the precipitate was filtered, and the filtrate was concentrated under reduced pressure to give crude product which was purified by column chromatography on silica gel (dichloromethane:MeOH, 100:1, 1% NH₃ H₂O) to give (3-amino-3-phenyl-propyl)-carbamic acid tert-butyl ester as an off-white solid. (Yield 1.7 g, 92%).

¹H NMR (300 MHz, CDCl₃): δ 7.31-7.18 (m, 5H), 6.82 (brs, 1H), 3.78-3.74 (m, 1H), 2.92 (brs, 2H), 1.82 (s, 2H), 1.63-1.61 (m, 2H), 1.37 (s, 9H). LC-MS: [M+H]⁺251.

### Example 4

### ((S)-3-Amino-3-phenyl-propyl)-carbamic acid tert-butyl ester

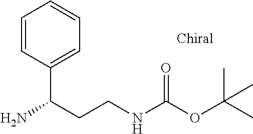

### Step A

### Racemic (3-hydroxy-3-phenyl-propyl)-carbamic acid tert-butyl ester

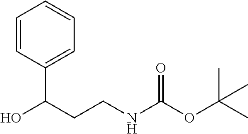

A solution of 3-oxo-3-phenylpropanenitrile (7.26 g, 50 mmol) in tetrahydrofuran (100 mL) was added, over 15 minutes, to an ice cooled solution of a 1.0 M solution of lithium aluminum hydride in tetrahydrofuran (100 mL). The mixture was stirred for 15 minutes at room temperature, then at 60° C. for 2 hours. The mixture was cooled in an ice bath and quenched by the dropwise addition of water (3.8 mL), followed by the dropwise addition of 4 M sodium hydroxide solution (3.8 mL), followed by the dropwise addition of water (11.4 mL). The mixture was stirred at room temperature for an additional 20 minutes, and the solids were then removed by suction filtration, rinsing the solids with tetrahydrofuran. The mixture was stirred overnight with BOC-anhydride (13 g, 59.6 mmol), concentrated under reduced pressure, and the residue purified by chromatography on silica gel, eluting with hexanes-ethyl acetate (70:30) to give racemic (3-hydroxy-3-phenyl-propyl)-carbamic acid tert-butyl ester. (Yield 7.9 g, 62.9%).

### Step B

### Racemic 3-(1,3-dioxo-1,3-dihydro-isoindol-2-yl)-3-phenyl-propyl]-carbamic acid tert-butyl ester

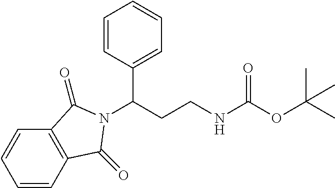

To a solution of racemic (3-hydroxy-3-phenyl-propyl)-carbamic acid tert-butyl ester (5.32 g, 36.1 mmol), and triphenylphosphine (9.48 g, 36.1 mmol) in tetrahydrofuran (200 mL) was added, over 10 minutes, diisopropyl azodicarboxylate (7.31 g, 36.1 mmol). After stirring at room temperature for 3 hours, the mixture was concentrated under reduced pressure and the residue purified by chromatography on silica gel, eluting with dichloromethane-ethyl acetate (90:10) to give pure racemic 3-(1,3-dioxo-1,3-dihydro-isoindol-2-yl)-3-phenyl-propyl-carbamic acid tert-butyl ester. (Yield 2.3 g, 16.7%).

### Step C

### Separation of (R)- and [(S)-3-(1,3-dioxo-1,3-dihydro-isoindol-2-yl)-3-phenyl-propyl]-carbamic acid tert-butyl ester

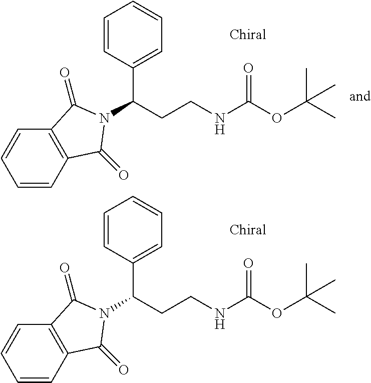

The separation of enantiomers of racemic [3-(1,3-dioxo-1,3-dihydro-isoindol-2-yl)-3-phenyl-propyl]-carbamic acid tert-butyl ester (2.3 g) was accomplished by chromatography under supercritical fluid chromatography (SFC), eluting with carbon dioxide as the carrier and 15% methanol as modifier (AD column), to give [(R)-3-(1,3-dioxo-1,3-dihydro-isoindol-2-yl)-3-phenyl-propyl]-carbamic acid tert-butyl ester as the first eluting peak (Yield 1.14 g), and [(S)-3-(1,3-dioxo-1,3-dihydro-isoindol-2-yl)-3-phenyl-propyl]-carbamic acid tert-butyl ester as the second eluting peak (Yield 1.13 g). Each enantiomer was obtained as a glass.

### Step D

### ((S)-3-Amino-3-phenyl-propyl)-carbamic acid tert-butyl ester

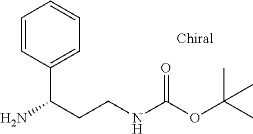

A solution of [(S)-3-(1,3-dioxo-1,3-dihydro-isoindol-2-yl)-3-phenyl-propyl]-carbamic acid tert-butyl ester (1.13 g, 2.98 mmol), hydrazine (0.93 mL, 29.8 mmol) in methanol (20 mL) was heated at reflux for 2 hours. The mixture was cooled, taken up in dichloromethane (400 mL) as was once with 1 M sodium hydroxide (85 mL), dried over anhydrous sodium sulfate plus a few pellets of sodium hydroxide, filtered and concentrated under reduced pressure to give ((S)-3-amino-3-phenyl-propyl)-carbamic acid tert-butyl ester, as an oil, which was used without further purification. (Yield 0.73 g, 92%).

### Example 5

### ((R)-3-Amino-3-phenyl-propyl)-carbamic acid tert-butyl ester

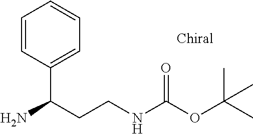

A solution of [(R)-3-(1,3-dioxo-1,3-dihydro-isoindol-2-yl)-3-phenyl-propyl]-carbamic acid tert-butyl ester (1.14 g, 3 mmol) (from Example 4 supra), hydrazine (0.94 mL, 30 mmol) in methanol (20 mL) was heated at reflux for 2 hours. The mixture was cooled, taken up in dichloromethane (400 mL) as was once with 1 M sodium hydroxide (85 mL), dried over anhydrous sodium sulfate plus a few pellets of sodium hydroxide, filtered and concentrated under reduced pressure to give ((R)-3-amino-3-phenyl-propyl)-carbamic acid tert-butyl ester, as an oil, which was used without further purification. (Yield 0.74 g, 92.6%).

### Example 6

### [3-Amino-3-(3-chloro-phenyl)-propyl]-carbamic acid tert-butyl ester

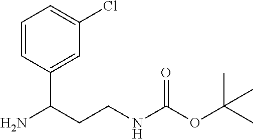

[3-Amino-3-(3-chloro-phenyl)-propyl]-carbamic acid tert-butyl ester was prepared in an analogous process to that described in Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-(3-chloro-phenyl)-propan-1-ol

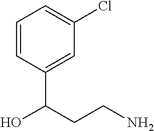

To a stirred suspension of LAH (16 g, 90 mmol) in dry THF (200 mL) was added a solution of 3-(3-chlorophenyl)-3-oxopropanenitrile (10.4 g, 270 mmol) in dry THF (200 mL) dropwise at 0° C. under nitrogen atmosphere. The mixture was warmed to 25° C. and then heated at 60° C. for 3 hours. After cooling to 0° C., a saturated solution of sodium hydroxide was added dropwise and extracted with ethyl acetate (200 mL). The solution was dried over anhydrous sodium sulfate and concentrated to dryness. The crude 3-amino-1-(3-chloro-phenyl)-propan-1-ol obtained was used in the next step without further purification. (Yield 14.5 g, crude).

LC-MS: [M+H]⁺186.

### Step B

### [3-(3-Chloro-phenyl)-3-hydroxy-propyl]carbamic acid tert-butyl ester

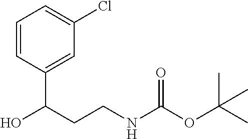

To a stirred solution of crude 3-amino-1-(3-chloro-phenyl)-propan-1-ol (29 g, 156 mmol) in THF (300 mL) was added Boc₂O (40.5 g, 187 mmol). After 0.5 hour, the mixture was concentrated to dryness. The residue was purified by column chromatography (ethyl acetate:petroleum ether, 1:20) to afford [3-(3-chloro-phenyl)-3-hydroxy-propyl]-carbamic acid tert-butyl ester. (Yield 23 g, 52%). LC-MS: [M+Na]⁺308.

### Step C

### [3-(3-Chloro-phenyl)-3-(1,3-dioxo-1,3-dihydro-isoindol-2-yl)-propyl]-carbamic acid tert-butyl ester

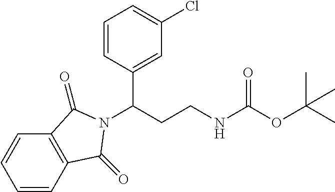

To a stirred solution of [3-(3-chloro-phenyl)-3-hydroxy-propyl]carbamic acid tert-butyl ester (12 g, 42 mmol), phthalimide (6.2 g, 42 mmol), and PPh₃ (14.3 g, 55 mmol) in THF (150 mL) was added DEAD (9.0 mL, 55 mmol) dropwise at about 5° C. After 1 hour, the mixture was concentrated to dryness. The residue was purified by column chromatography (ethyl acetate:petroleum ether, 1:8) to afford [3-(3-chloro-phenyl)-3-(1,3-dioxo-1,3-dihydro-isoindol-2-yl)-propyl]-carbamic acid tert-butyl ester. (Yield 15.65 g, 90%).

LC-MS: [M+H]⁺415.

### Step D

### [3-Amino-3-(3-chloro-phenyl)-propyl]-carbamic acid tert-butyl ester

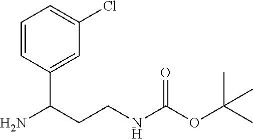

To a stirred solution of [3-(3-chloro-phenyl)-3-(1,3-dioxo-1,3-dihydro-isoindol-2-yl)-propyl]-carbamic acid tert-butyl ester (0.15 g, 0.36 mmol) in THF (2 mL) and methanol (2 mL) was added hydrazine hydrate (0.18 g, 3.6 mmol). The mixture was heated at 55° C. for 2 hours. Then the reaction mixture was concentrated and extracted with ethyl acetate (10 mL). The organic mixture was washed with water (3×1 mL), brine (1 mL), dried over anhydrous sodium sulfate and concentrated to dryness. The residue was purified by column chromatography (methanol:dichloromethane, 1:100) to afford [3-amino-3-(3-chloro-phenyl)-propyl]-carbamic acid tert-butyl ester. (Yield 0.061 g, 60%). LC-MS: [M+H]⁺285.

### Example 7

### [(S)-3-Amino-3-(3-chloro-phenyl)-propyl]-carbamic acid tert-butyl ester

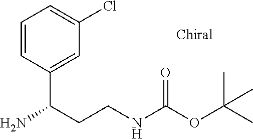

[3-Amino-3-(3-chloro-phenyl)-propyl]-carbamic acid tert-butyl ester (1 g, 3.51 mmol) (from Example 6 supra) was purified by supercritical fluid chromatography [(R,R)-WHELK-O1 column, Regis Technologies, 20% methanol in carbon dioxide as solvent] to give [(S)-3-amino-3-(3-chloro-phenyl)-propyl]-carbamic acid tert-butyl ester. (Yield 0.47 g).

### Example 8

### [2-Amino-2-(3-chloro-phenyl)-ethyl]-carbamic acid tert-butyl ester

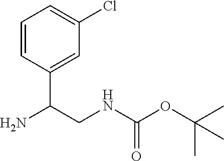

[2-Amino-2-(3-chloro-phenyl)-ethyl]-carbamic acid tert-butyl ester was prepared in an analogous process to that described in Seefeld, M. A.; Rouse, M. B.; Heerding, D. A.; Peace, S.; Yamashita, D. S.; McNulty, K. C. WO 2008/098104, Aug. 14, 2008.

### Step A

### (3-Chloro-phenyl)-hydroxy-acetonitrile

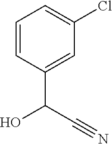

To a stirred suspension of KCN (5.04 g, 78 mmol) in methanol (20 mL) was added 3-chlorobenzaldehyde (7.0 g, 50 mmol) at 0° C. under nitrogen atmosphere. Then acetic acid (4.4 mL) was added dropwise at 0° C. After 30 minutes, the mixture was warmed to 15° C. and stirred for 5 hours. Then the reaction mixture was concentrated to dryness and extracted with ethyl acetate (200 mL). The organic solution was washed with water (3×25 mL), brine (25 mL), dried over anhydrous sodium sulfate and concentrated to dryness. The resulting residue was purified by column chromatography (ethyl acetate:petroleum ether, 1:15) to afford (3-chloro-phenyl)-hydroxy-acetonitrile. (Yield 8.2 g, 97%).

LC-MS: [M+Na]⁺190.

### Step B

### 2-Amino-1-(3-chloro-phenyl)-ethanol

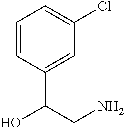

To a stirred suspension of LAH (2.36 g, 59 mmol) in dry THF (70 mL) was added a solution of (3-chloro-phenyl)-hydroxy-acetonitrile (4.0 g, 24 mmol) in dry THF (55 mL) dropwise at 0° C. under nitrogen atmosphere. The mixture was warmed to 25° C. and then heated at 60° C. for 2 hours. After cooling to 0° C., a saturated solution of sodium hydroxide was added dropwise and extracted with dichloromethane (200 mL). The organic solution was dried over anhydrous sodium sulfate and concentrated to dryness. The residue was purified by column chromatography (methanol:dichloromethane, 1:10) to afford 2-amino-1-(3-chloro-phenyl)-ethanol. (Yield 2.86 g, 70%).

LC-MS: [M+H]⁺172.

### Step C

### [2-(3-Chloro-phenyl)-2-hydroxy-ethyl]carbamic acid tert-butyl ester

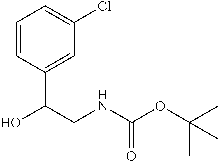

To a stirred solution of 2-amino-1-(3-chloro-phenyl)-ethanol (2.86 g, 16.7 mmol) in THF (100 mL) was added Boc₂O (4.3 g, 20 mmol). After 1 hour, the mixture was concentrated to dryness. The residue was purified by column chromatography (methanol:dichloromethane, 1:100) to afford [2-(3-chloro-phenyl)-2-hydroxy-ethyl]-carbamic acid tert-butyl ester. (Yield 3.9 g, 72%). LC-MS: [M+Na]⁺294.

### Step D

### [2-(3-Chloro-phenyl)-2-(1,3-dioxo-1,3-dihydro-isoindol-2-yl)-ethyl]-carbamic acid tert-butyl

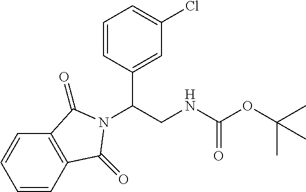

To a stirred solution of [2-(3-chloro-phenyl)-2-hydroxy-ethyl]-carbamic acid tert-butyl ester (20 g, 73.5 mmol), phthalimide (11.1 g, 73.5 mmol) and PPh₃ (25.1 g, 95.5 mmol) in THF (500 mL) was added DEAD (11.4 mL, 95.5 mmol) dropwise at −5 to 0° C. The reaction mixture was stirred at room temperature for 3 hours. Then the mixture was concentrated to dryness. The residue was purified by column chromatography (ethyl acetate:petroleum ether, 1:10) to afford [2-(3-chloro-phenyl)-2-(1,3-dioxo-1,3-dihydro-isoindol-2-yl)-ethyl]-carbamic acid tert-butyl ester. (Yield 20 g, 69%). LC-MS: [M+H]⁺401.

### Step E

### [2-Amino-2-(3-chloro-phenyl)-ethyl]carbamic acid tert-butyl ester

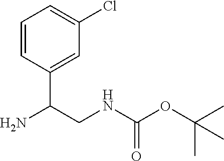

To a stirred solution of [2-(3-chloro-phenyl)-2-(1,3-dioxo-1,3-dihydro-isoindol-2-yl)-ethyl]-carbamic acid tert-butyl ester (2.5 g, 6.2 mmol) in THF (10 mL) and methanol (10 mL) was added hydrazine hydrate (3.1 g, 62 mmol). The mixture was heated at 55° C. for 1 hour. Then it was concentrated to dryness, dissolved in H₂O (5 mL) and extracted with ethyl acetate (50 mL). The organic mixture was concentrated and purified by column chromatography (methanol:dichloromethane, 1:100) to afford [2-amino-2-(3-chloro-phenyl)-ethyl]-carbamic acid tert-butyl ester. (Yield 1.325 g, 79%).

LC-MS: [M+H]⁺271.

### Example 9

### [(R)-2-Amino-2-(3-chloro-phenyl)-ethyl]-carbamic acid tert-butyl ester

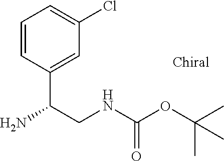

[2-Amino-2-(3-chloro-phenyl)-ethyl]carbamic acid tert-butyl ester (1 g, 3.69 mmol) (from Example 8 supra) was purified by supercritical fluid chromatography [(R,R)-WHELK-O1 column, Regis Technologies, 20% methanol in carbon dioxide as solvent] to give [(R)-2-amino-2-(3-chloro-phenyl)-ethyl]-carbamic acid tert-butyl ester. (Yield 0.47 g).

### Example 10

### [(S)-2-Amino-2-(3-chloro-phenyl)-ethyl]carbamic acid tert-butyl ester

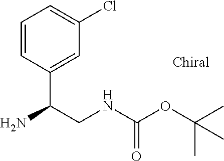

[2-Amino-2-(3-chloro-phenyl)-ethyl]carbamic acid tert-butyl ester (1 g, 3.69 mmol) (from Example 8 supra) was purified by supercritical fluid chromatography [(R,R)-WHELK-O1 column, Regis Technologies, 20% methanol in carbon dioxide as solvent] to give [(S)-2-amino-2-(3-chloro-phenyl)-ethyl]-carbamic acid tert-butyl ester. (Yield 0.50 g).

### Example 11

### (3-Amino-3-thiophen-3-yl-propyl)-carbamic acid tert-butyl ester

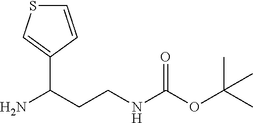

(3-Amino-3-thiophen-3-yl-propyl)-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-thiophen-3-yl-propan-1-ol

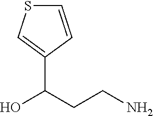

To a stirred suspension of LAH (1.45 g, 38.1 mmol) in dry THF (120 mL) was added a solution of 3-oxo-3-(thiophen-3-yl)propanenitrile (4.8 g, 31.8 mmol) in dry THF (40 mL) dropwise at 0° C. under nitrogen atmosphere. The mixture was warmed to 25° C. and then heated at 65° C. for 6 hours. After cooling to 0° C., a saturated solution of sodium hydroxide (2 mL) was added dropwise and the mixture was filtered. The filtrate was concentrated to dryness to give crude 3-amino-1-thiophen-3-yl-propan-1-ol which was used in next step without further purification.

¹H NMR (300 MHz, CDCl₃): δ 7.29-7.26 (m, 2H), 7.05 (dd, 1H, J₁=4.8 Hz, J₂=1.2 Hz), 5.04 (dd, 1H, J₁=8.1 Hz, J₂=3.0 Hz), 3.10-3.05 (m, 2H), 1.82-1.77 (m, 2H).

### Step B

### (3-Hydroxy-3-thiophen-3-yl-propyl)-carbamic acid tert-butyl ester

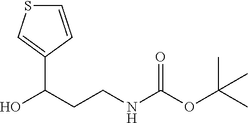

To a stirred solution of crude 3-amino-1-thiophen-3-yl-propan-1-ol (23 g) in THF (100 mL) was added Boc₂O (31.6 g, 146.3 mmol). The mixture was stirred at room temperature for 1 hour and then concentrated to dryness. The residue was purified by column chromatography (ethyl acetate:petroleum ether, 1:10) to afford (3-hydroxy-3-thiophen-3-yl-propyl)-carbamic acid tert-butyl ester. (Yield 21.5 g, 51% for two steps).

¹H NMR (300 MHz, CDCl₃): δ 8.08-8.06 (m, 1H), 7.55-7.53 (m, 1H), 7.34-7.30 (m, 1H), 5.10 (s, 1H), 3.52-3.48 (m, 2H), 3.13-3.09 (m, 2H), 1.42 (s, 9H). LC-MS: [M+Na]⁺280.

### Step C

### 3-(1,3-Dioxo-1,3-dihydro-isoindol-2-yl)-3-thiophen-3-yl-propyl]-carbamic acid tert-butyl ester

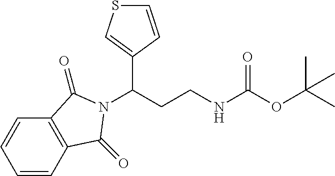

To a stirred solution of (3-hydroxy-3-thiophen-3-yl-propyl)-carbamic acid tert-butyl ester (21.5 g, 83.6 mmol), phthalimide (12.3 g, 83.6 mmol), and PPh₃ (28.5 g, 108.6 mmol) in THF (400 mL) was added DEAD (17.6 mL, 108.6 mmol) dropwise at 25° C. The mixture was stirred at room temperature for 14 hours, then concentrated. The residue was purified by column chromatography (ethyl acetate:petroleum ether, 1:6) to afford 3-(1,3-dioxo-1,3-dihydro-isoindol-2-yl)-3-thiophen-3-yl-propyl]-carbamic acid tert-butyl ester. (Yield 12 g, 38%).

¹H NMR (300 MHz, CDCl₃): δ 7.82-7.77 (m, 2H), 7.72-7.68 (m, 2H), 7.36 (d, 1H, J=1.8 Hz), 7.26-7.18 (m, 2H), 5.50 (dd, 1H, J₁=9.6 Hz, J₂=6 Hz), 4.65 (br, 1H), 3.24-3.07 (m, 2H), 2.72-2.67 (m, 1H), 2.47-2.40 (m, 1H), 1.40 (s, 9H). LC-MS: [M+H-Boc]⁺287.

### Step D

### (3-Amino-3-thiophen-3-yl-propyl)-carbamic acid tert-butyl ester

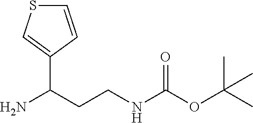

To a stirred solution of 3-(1,3-dioxo-1,3-dihydro-isoindol-2-yl)-3-thiophen-3-yl-propyl]-carbamic acid tert-butyl ester (12 g, 31.1 mmol) in methanol (150 mL) was added hydrazine hydrate (18 mL, 85% aqueous). The mixture was heated at reflux for 14 hours. After cooling to room temperature, the reaction mixture was filtered. The filtrate was concentrated and the residue was purified by column chromatography (methanol:dichloromethane, 1:50 to 1:20, 0.1% NH₃ H₂O) to afford (3-amino-3-thiophen-3-yl-propyl)-carbamic acid tert-butyl ester. (Yield 7.6 g, 95%).

¹H NMR (300 MHz, CDCl₃): δ 7.49 (s, 1H), 7.25-7.08 (m, 2H), 6.82 (brs, 1H), 3.85 (t, 1H, J=6.0 Hz), 3.18-2.95 (m, 4H), 1.75-1.62 (m, 2H), 1.37 (s, 9H). LC-MS: [M+H]⁺257.

### Example 12

### 6,7-Dimethoxy-2-oxo-1,2-dihydro-quinoline-3-carboxylic acid methyl ester

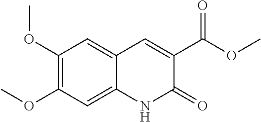

### Step A

### 2-Amino-4,5-dimethoxybenzaldehyde

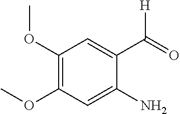

6-Nitroveratraldehyde (tech, 80%, 5.28 g, 20 mmol) (Aldrich) was dissolved in mixture of ethanol (50 mL) and acetic acid (50 mL) with warming. Water (30 mL) was then added followed by conc. hydrochloric acid (0.12 g). Iron powder (4.2 g, 75 mmol) was added with vigorous stirring and mixture was heated at reflux for 30 minutes. Mixture was filtered through Celite while hot. Filter cake was washed with ethanol. Combined filtrate and washing was diluted with water (300 mL) and extracted with ethyl acetate (3×300 mL). Organic layers were washed with saturated aqueous sodium bicarbonate solution (2×200 mL), water (200 mL) and brine (200 mL) and then combined. Combined organic layer was dried (MgSO₄), filtered, and concentrated to give crude 2-amino-4,5-dimethoxybenzaldehyde as brown oil which was used in next step without further purification. (TLC ethyl acetate-dichloromethane 3:7 showed it to be one major spot.) (Yield 2.72 g, 75%).

### Step B

### 6,7-Dimethoxy-2-oxo-1,2-dihydro-quinoline-3-carboxylic acid methyl ester

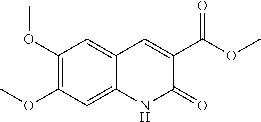

A mixture of 2-amino-4,5-dimethoxybenzaldehyde (crude, 2.72 g, 15 mmol), dimethyl malonate (5.50 g, 41.6 mmol) (Aldrich), piperidine (3.60 g, 42.3 mmol) (Aldrich), and acetic acid (0.1 g, 1.7 mmol) in methanol (60 mL) was heated at 60° C. for 24 hours. After cooling crystals formed was collected and washed with methanol to give 6,7-dimethoxy-2-oxo-1,2-dihydro-quinoline-3-carboxylic acid methyl ester as yellow crystals. Filtrate was concentrated to give a thick brown oil. This was suspended in small amount of methanol and precipitate collected by filtration and washing with methanol to give second crop of product. (Yield 2.34 g, 59%). HR-MS (ES⁺) m/z Calculated for C₁₃H₁₄NO₅ ([M+H]⁺): 264.0867. Found: 264.0866.

### Example 13

### 3-Amino-N-benzyl-4-methyl-benzamide

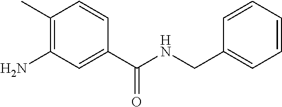

### Step A

### N-Benzyl-4-methyl-3-nitro-benzamide

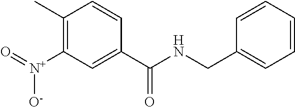

A solution of 4-methyl-3-nitrobenzoyl chloride (2.0 g, 10 mmol) (Aldrich) in ether (20 mL) was added dropwise to a solution of benzylamine (1.08 g, 10 mmol) (Aldrich), triethylamine (1.22 g, 12 mmol) and 4-dimethylamino-pyridine (60 mg, 0.5 mmol) (Fluka) in ether (80 mL) at room temperature with magnetic stirring. After 18 hours, mixture was diluted with water (100 mL). After thorough mixing, layers were separated. Organic layer was washed with 1 N aqueous hydrochloric acid, saturated aqueous sodium bicarbonate solution and brine (100 mL each). Aqueous layers were back washed with ether (100 mL). Ether layers were then combined, dried (MgSO₄), filtered and concentrated under reduced pressure to give N-benzyl-4-methyl-3-nitro-benzamide as white crystalline solid. (Yield 2.42 g, 89.5%).

### Step B

### 3-Amino-N-benzyl-4-methyl-benzamide

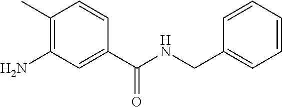

Iron powder (1.5 g, 26.9 mmol) was added slowly to a suspension of benzyl 4-methyl-3-nitro-benzamide (2.42 g, 8.95 mmol) in mixture of acetic acid (20 mL), ethanol (20 mL) and water (10 mL). Mixture was heated in an oil bath at 100° C. for 30 minutes with magnetic stirring. After cooling to room temperature, mixture was diluted with ethyl acetate (150 mL) and filtered through a pad of Celite. Filtrate was mixed thoroughly with mixture of conc. aqueous ammonium hydroxide (40 mL) and ice (100 g). Precipitate was filtered off and layers separated. Organic layer was washed with saturated aqueous sodium bicarbonate solution and brine (100 mL each). Aqueous layers were back washed with ethyl acetate (200 mL). Organic layers were then combined, dried (MgSO₄), filtered and concentrated to give 3-amino-N-benzyl-4-methyl-benzamide as yellow crystalline solid. This was used in next step without further purification. (Yield 2.27 g, 106%).

### Example 14

### 3-Amino-N-benzyl-4-chloro-benzamide

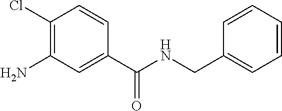

### Step A

### Benzyl 4-chloro-3-nitro-benzamide

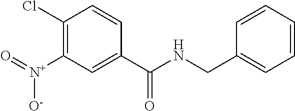

A solution of 4-chloro-3-nitrobenzoyl chloride (2.2 g, 10 mmol) (Aldrich) in ether (20 mL) was added dropwise to a solution of benzylamine (1.08 g, 10 mmol) (Aldrich), triethylamine (1.22 g, 12 mmol) and 4-dimethylamino-pyridine (60 mg, 0.5 mmol) (Fluka) in ether (80 mL) at room temperature with magnetic stirring. After 18 hours, mixture was diluted with water (100 mL). After thorough mixing, layers were separated. Organic layer was washed with 1 N aqueous hydrochloric acid, saturated aqueous sodium bicarbonate solution and brine (100 mL each). Aqueous layers were back washed with ether (100 mL). Ether layers were then combined, dried (MgSO₄), filtered and concentrated under reduced pressure to give benzyl 4-chloro-3-nitro-benzamide as white crystalline solid. (Yield 2.812 g, 96.7%).

### Step B

### 3-Amino-N-benzyl-4-chloro-benzamide

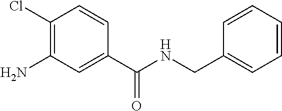

Iron powder (1.62 g, 29 mmol) was added slowly to a suspension of benzyl 4-chloro-3-nitro-benzamide (2.81 g, 9.7 mmol) in mixture of acetic acid (20 mL), ethanol (20 mL) and water (10 mL). Mixture was heated in an oil bath at 100° C. for 30 minutes with magnetic stirring. After cooling to room temperature, mixture was diluted with ethyl acetate (150 mL) and filtered through a pad of Celite. Filtrate was mixed thoroughly with mixture of conc. aqueous ammonium hydroxide (40 mL) and ice (100 g). Precipitate was filtered off and layers separated. Organic layer was washed with saturated aqueous sodium bicarbonate solution and brine (100 mL each). Aqueous layers were back washed with ethyl acetate (200 mL). Organic layers were then combined, dried (MgSO₄), filtered and concentrated to give crude product as yellow crystalline solid. This was re-crystallized from ethyl acetate-heptane to give 3-amino-N-benzyl-4-chloro-benzamide as pale yellow crystals. (Yield 2.26 g, 89.7%).

### Example 15

### 6,7-Dimethoxy-2-oxo-1,2-dihydro-quinoline-3-carboxylic acid

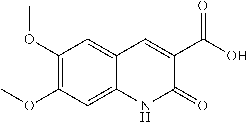

A mixture of 6,7-dimethoxy-2-oxo-1,2-dihydro-quinoline-3-carboxylic acid methyl ester (2.24 g, 8.5 mmol) (from Example 12 supra) and sodium hydroxide (1.04 g, 25.5 mmol) in methanol (25 mL) and water (25 mL) was heated at 65° C. for 18 hours. After cooling, mixture was diluted with water and concentrated under reduced pressure to remove most of the methanol. Clear solution was obtained. This was acidified with 2 N aqueous hydrochloric acid. White precipitate formed was collected by filtration and washed with water and dried in vacuum oven to give crude 6,7-dimethoxy-2-oxo-1,2-dihydro-quinoline-3-carboxylic acid as white powder. (Yield 2.10 g, 99%).

### Example 16

### 4-Methyl-3-[(2-oxo-1,2-dihydro-quinoline-3-carbonyl)-amino]-benzoic acid methyl ester

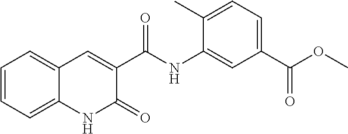

### HATU Method

Triethylamine (65 mg, 0.64 mmol) (Acros) was added to a mixture of 2-quinolone-3-carboxylic acid (100 mg, 0.53 mmol) (Maybridge)) and HATU (241 mg, 0.64 mmol) (Aldrich) in DMF (5.0 mL) at room temperature. Mixture was stirred until clear solution was obtained (light brown). 3-Amino-p-toluic acid methyl ester (0.12 g, 0.70 mmol) (TCI) was added. Mixture was stirred for another 20 hours. Precipitate was formed. Water (50 mL), aqueous saturated sodium bicarbonate solution (10 mL), and ethyl acetate (25 mL) were added. After thorough mixing, white precipitate was collected by filtration, washed with water and ethyl acetate and recrystallized from hot DMF-ethyl acetate-hexanes to give 4-methyl-3-[(2-oxo-1,2-dihydro-quinoline-3-carbonyl)-amino]-benzoic acid methyl ester fine yellow needles. (Yield 0.16 g, 90.0%).

HR-MS (ES⁺) m/z Calculated for C₁₉H₁₇N₂O₄ ([M+H]⁺): 337.1183. Found: 337.1183.

### Example 17

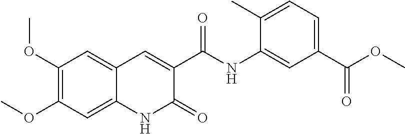

### Acid Chloride Method

To a suspension of the 6,7-dimethoxy-2-oxo-1,2-dihydro-quinoline-3-carboxylic acid (4) (100 mg, 0.4 mmol) (from Example 15 supra) in dichloromethane (5 mL) was added oxalyl chloride (61 mg, 0.48 mmol) (Aldrich) followed by a catalytic amount of DMF in dichloromethane (100 μL). The reaction was then stirred at room temperature for 4 hours. After this time, Et₃N (137 mg, 1.35 mmol) was added followed by a solution of the 3-amino-p-toluic acid methyl ester (0.11 g, 0.675 mmol) (TCI) in dichloromethane (1 mL) and the reaction was stirred at room temperature for 18 h. After this time the mixture was concentrated to dryness and the residue suspended in water and the solid collected by filtration and washed with water then methanol. The collected solid was dissolved in DMSO (2 mL) (with gentle heating) then water (0.5 mL) was added and the precipitate was collected by filtration and washed with methanol to give 3-[(6,7-dimethoxy-2-oxo-1,2-dihydro-quinoline-3-carbonyl)-amino]-4-methyl-benzoic acid methyl ester. HR-MS (ES⁺) m/z Calculated for C₂₁H₂₁N₂O₆ ([M+H]⁺): 397.1394. Found: 397.1395.

### Example 18

### 3-[(6,7-Dimethoxy-2-oxo-1,2-dihydro-quinoline-3-carbonyl)-amino]-4-methyl-benzoic acid

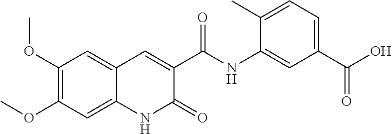

To a solution of 3-[(6,7-dimethoxy-2-oxo-1,2-dihydro-quinoline-3-carbonyl)-amino]-4-methyl-benzoic acid methyl ester (1.0 g, 2.5 mmol) (from Example 17 supra) in MeOH (20 mL) NaOH (1.0 g, 25.2 mmol) and water (40 mL) were added and the reaction mixture was heated at 60° C. for 4 hours. Then, to the reaction mixture was added 4 N HCl solution which resulted in the formation of a paste. Filtration under vacuum, the paste was then washed with MeOH and dried under air overnight to afford 3-[(6,7-dimethoxy-2-oxo-1,2-dihydro-quinoline-3-carbonyl)-amino]-4-methyl-benzoic acid. (Yield 0.8 g, 83%). HR-MS (ES⁺) m/z Calculated for C₂₀H₁₉N₂O₆ ([M+H]⁺): 383.1238. Found: 383.1238.

### Example 19

### 4-Chloro-3-[(6,7-dimethoxy-2-oxo-1,2-dihydro-quinoline-3-carbonyl)-amino]-benzoic acid methyl ester

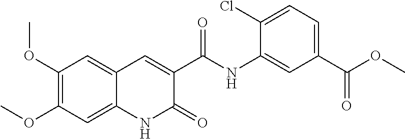

4-Chloro-3-[(6,7-dimethoxy-2-oxo-1,2-dihydro-quinoline-3-carbonyl)-amino]-benzoic acid methyl ester was synthesized in a manner similar to Example 16 with 6,7-dimethoxy-2-oxo-1,2-dihydro-quinoline-3-carboxylic acid (from Example 15 supra) and methyl 3-amino-4-chlorobenzoate (TCI) as reactants. (Yield 0.20 g, 59.8%).

HR-MS (ES⁺) m/z Calculated for C₂₀H₁₈ClN₂O₆ ([M+H]⁺): 417.0848. Found: 417.0848.

### Example 20

### 6,7-Dimethoxy-2-oxo-1,2-dihydro-quinoline-3-carboxylic acid (2-chloro-5-dimethylcarbamoyl-phenyl)-amide

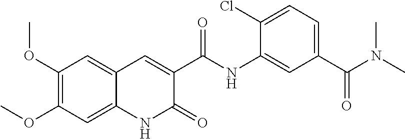

### Step A

### N,N-Dimethyl-3-nitro-4-chloro-benzamide

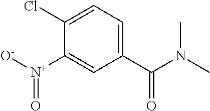

A solution of 4-chloro-3-nitrobenzoyl chloride (11.0 g, 50 mmol) (Aldrich) in ether (100 mL) was added dropwise to a mixture of dimethylamine (2.0 M in THF, 50 mL, 100 mmol) (Aldrich), sodium bicarbonate (8.5 g, 101 mmol), ether (50 mL) and water (100 mL) with cooling in ice bath and magnetic stirring. Mixture was allowed to warm up to room temperature slowly. After stirring at room temperature for 2 hours, layers were separated. Organic layer was washed with water, 1 N aqueous hydrochloric acid and brine. Aqueous layers were back washed with ether. Ether layers were combined, dried (MgSO₄), filtered, and concentrated to give crude N,N-dimethyl-3-nitro-4-chloro-benzamide which was used without further purification. Yield was not determined.

### Step B

### 3-Amino-4-chloro-N,N-dimethyl-benzamide

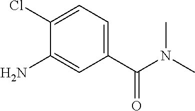

N,N-Dimethyl-3-nitro-4-chloro-benzamide (6.6 g, 28.9 mmol) was dissolved in mixture of ethanol (75 mL) and acetic acid (750 mL). Water (45 mL) was then added followed by conc. hydrochloric acid (0.18 g). Iron powder (6.06 g, 108.6 mmol) was added with vigorous stirring and mixture was heated at reflux for 30 minutes. Mixture was filtered through Celite while hot. Filter cake was washed with ethanol. Combined filtrate and washing was diluted with water (300 mL) and extracted with ethyl acetate (3×300 mL). Organic layers were washed with saturated aqueous sodium bicarbonate solution (2×200 mL), water (200 mL) and brine (200 mL) and then combined. Combined organic layer was dried (MgSO₄), filtered, and concentrated to give crude 3-amino-4-chloro-N,N-dimethyl-benzamide as pale yellow oil which crystallized on standing. (Yield 5.50 g, 95.9%).

### Step C

### 6,7-Dimethoxy-2-oxo-1,2-dihydro-quinoline-3-carboxylic acid (2-chloro-5-dimethylcarbamoyl-phenyl)-amide

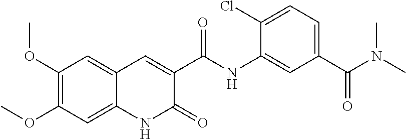

6,7-Dimethoxy-2-oxo-1,2-dihydro-quinoline-3-carboxylic acid (2-chloro-5-dimethylcarbamoyl-phenyl)-amide was synthesized in a manner similar to Example 16 with 6,7-dimethoxy-2-oxo-1,2-dihydro-quinoline-3-carboxylic acid (from Example 15 supra) and 3-amino-4-chloro-N,N-dimethyl-benzamide as reactants. (Yield 0.26 g, 75.4%). HR-MS (ES⁺) m/z Calculated for C₂₁H₂₁ClN₃O₅ ([M+H]⁺): 430.1164. Found: 430.1165.

### Example 21

### 6,7-Dimethoxy-2-oxo-1,2-dihydro-quinoline-3-carboxylic acid (3-methyl-pyridin-4-yl)-amide

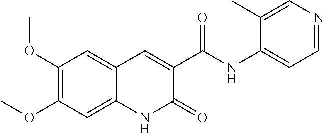

To a solution of 6,7-dimethoxy-2-oxo-1,2-dihydro-quinoline-3-carboxylic acid (0.1 g, 0.40 mmol) (from Example 15 supra) in DMF (5 mL) was added HATU (0.182 g, 0.48 mmol) (Aldrich), triethylamine (0.070 mL, 0.48 mmol) (Aldrich) and 4-amino-3-methylpyridine (0.056 g, 0.52 mmol) (Chontech). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid (3-methyl-pyridin-4-yl)-amide. (Yield 0.07 g, 52%). HR-MS (ES⁺) m/z Calculated for C₁₈H₁₈N₃O₄ ([M+H]⁺): 340.1292. Found: 340.1291.

### Example 22

### 6,7-Dimethoxy-2-oxo-1,2-dihydro-quinoline-3-carboxylic acid [2-methyl-5-(1H-tetrazol-5-yl)-phenyl]-amide

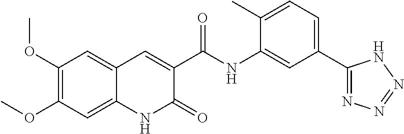

6,7-Dimethoxy-2-oxo-1,2-dihydro-quinoline-3-carboxylic acid [2-methyl-5-(1H-tetrazol-5-yl)-phenyl]-amide was prepared from 6,7-dimethoxy-2-oxo-1,2-dihydro-quinoline-3-carboxylic acid (0.1 g, 0.403 mmol) (from Example 15 supra) with 2-methyl-5-(2H-tetrazol-5-yl)-phenylamine (0.094 g, 0.536 mmol) (CHEMBRDG BB) by following the method of Example 16 using DIPEA instead of triethylamine. (Yield 105 mg, 64%).

¹H NMR (DMSO-d₆): δ 12.46 (br s, 1H), 12.17 (br s, 1H), 8.95 (s, 1H), 8.90 (s, 1H), 7.63 (d, 1H), 7.55 (s, 1H), 7.25 (d, 1H), 6.98 (s, 1H), 3.87 (s, 3H), 3.82 (s, 3H), 2.36 (s, 3H). (One NH not detected). HR-MS (ES⁺) m/z Calculated for C₂₀H₁₉N₆O₄ ([M+H]⁺): 407.1463. Found: 407.1462

### Example 23

### 3-ο[7-Methoxy-6-(2-methoxy-ethoxy)-2-oxo-1,2-dihydro-quinoline-3-carbonyl]-amino}-4-methyl-benzoic acid methyl ester

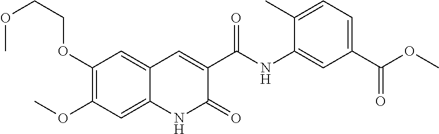

### Step A

### 4-Methoxy-3-(2-methoxy-ethoxy)-benzaldehyde

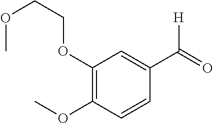

To a solution of 3-hydroxy-4-methoxy-benzaldehyde (10 g, 65.73 mmol) (Aldrich) in DMF (100 mL), 1-chloro-2-methoxy-ethane (6.83 g, 6.6 mL, 72.30 mmol) (Aldrich) was added, followed by K₂CO₃ (31.80 g, 230 mmol), potassium iodide (1 g) (Aldrich), and heated at 85° C. overnight. The reaction was diluted with EtOAc (600 mL), washed with water (2×500 mL), brine, and dried with anhydrous sodium sulfate. Concentration gave 4-methoxy-3-(2-methoxy-ethoxy)-benzaldehyde as an oil which was used in next step without purification. (Yield 14.5 g).

¹H NMR (CDCl₃): δ 9.84 (s, 1H), 7.46 (m, 1H), 7.42 (s, 1H), 6.97 (m, 1H), 4.23 (m, 2H), 3.94 (s, 3H), 3.82 (m, 2H), 3.45 (s, 3H).

### Step B

### 4-Methoxy-5-(2-methoxy-ethoxy)-2-nitro-benzaldehyde

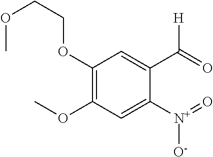

To a solution of 4-methoxy-3-(2-methoxy-ethoxy)-benzaldehyde (5 g, 23.78 mmol) in acetic acid (18 mL) cooled in ice bath, conc. nitric acid (7.5 mL) (Aldrich) was added and stirred for 5 mins. Potassium nitrate (2.35 g, 23.2 mmol) (Aldrich) was added followed by conc. H₂SO₄ (3 mL) (J. T. Baker). The reaction was allowed to warm to room temperature and stirred for 4 hours. A solid was formed. Water (20 mL) was added to the mixture. The mixture was filtered, and washed with water (3×50 mL). The solid was collected and dried under reduced pressure overnight to give 4-methoxy-5-(2-methoxy-ethoxy)-2-nitro-benzaldehyde. (Yield 4.57 g, 75%).

¹H NMR (DMSO-d₆): δ 10.16 (s, 1H), 7.68 (s, 1H), 7.36 (s, 1H), 4.27 (m, 2H), 3.95 (s, 3H), 3.67 (m, 2H), 3.29 (s, 3H).

### Step C

### 7-Methoxy-6-(2-methoxy-ethoxy)-2-oxo-1,2-dihydro-quinoline-3-carboxylic acid methyl ester

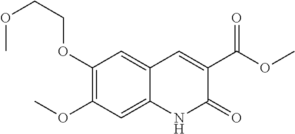

To a suspension of 4-methoxy-5-(2-methoxy-ethoxy)-2-nitro-benzaldehyde (1 g, 3.92 mL) in a mixture of MeOH (50 mL) and EtOAc (50 mL), was added 10% Pd/C (100 mg) (Aldrich). The mixture was agitated under 30 Psi H₂ for 1 hour. The reaction mixture was filtered through a Celite pad, and washed with MeOH. Concentration of the filtrate gave an oil which was used in next step without further purification.

To the above obtained oil in MeOH (40 mL), malonic acid dimethyl ester (0.933 g, 0.81 mL, 7.06 mmol) (Aldrich) was added, followed by piperidine (0.601 g, 0.7 mL, 7.06 mmol) (Aldrich), and AcOH (0.05 mL). The reaction mixture was heated at 60° C. overnight. The solvent was removed under reduced pressure. Purification by flash chromatography (ISCO system, 10% MeOH in dichloromethane) provided 7-methoxy-6-(2-methoxy-ethoxy)-2-oxo-1,2-dihydro-quinoline-3-carboxylic acid methyl ester as a brown solid. (Yield 0.31 g, 26% for two steps). ¹H NMR (DMSO-d₆): δ 11.85 (br s, 1H), 8.42 (s, 1H), 7.35 (s, 1H), 6.81 (s, 1H), 4.06 (m, 2H), 3.82 (s, 3H), 3.74 (s, 3H), 3.65 (m, 2H), 3.27 (s, 3H).

### Step D

### 7-Methoxy-6-(2-methoxy-ethoxy)-2-oxo-1,2-dihydro-quinoline-3-carboxylic acid

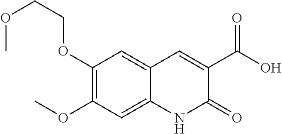

To a solution of 7-methoxy-6-(2-methoxy-ethoxy)-2-oxo-1,2-dihydro-quinoline-3-carboxylic acid methyl ester (2.45 g, 7.97 mmol) in MeOH (80 mL) was added NaOH (1.59 g, 39.9 mmol) and H₂O (8 mL) and the mixture was heated at 60° C. for 4 hours. The reaction was acidified to pH=3, filtered, washed with water, and dried under reduced pressure overnight to give 7-methoxy-6-(2-methoxy-ethoxy)-2-oxo-1,2-dihydro-quinoline-3-carboxylic acid. (Yield 2.16 g, 92%). ¹H NMR (DMSO-d₆): δ 14.93 (br s, 1H), 13.05 (br s, 1H), 8.79 (s, 1H), 7.55 (s, 1H), 6.99 (s, 1H), 4.13 (m, 2H), 3.90 (s, 3H), 3.71 (m, 2H), 3.32 (s, 3H).

### Step E

### 3-{[7-Methoxy-6-(2-methoxy-ethoxy)-2-oxo-1,2-dihydro-quinoline-3-carbonyl]-amino}-4-methyl-benzoic acid methyl ester

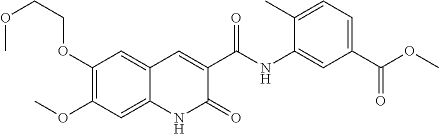

To a suspension of 7-methoxy-6-(2-methoxy-ethoxy)-2-oxo-1,2-dihydro-quinoline-3-carboxylic acid (0.08 g, 0.273 mmol) in DMF (4 mL), HATU (0.125 g, 0.327 mmol) (Aldrich) was added, followed by DIPEA (42 mg, 0.059 mL, 0.326 mmol) (Aldrich). The mixture was stirred for 5 mins. The reaction turned into a clear solution. 3-Amino-4-methyl-benzoic acid methyl ester (0.06 g, 0.363 mmol) (TCI) was added and stirred at room temperature overnight. The reaction was diluted with EtOAc (150 mL), washed with water (50 mL), brine, dried with anhydrous sodium sulfate, and concentrated to give an oil. This oil was purified by preparative HPLC to give 3-{[7-methoxy-6-(2-methoxy-ethoxy)-2-oxo-1,2-dihydro-quinoline-3-carbonyl]-amino}-4-methyl-benzoic acid methyl ester as a yellow solid. (Yield 0.037 g, 31%).

¹H NMR (DMSO-d₆): δ 12.47 (br s, 1H), 12.36 (br s, 1H), 9.01 (s, 1H), 8.86 (s, 1H), 7.62 (d, 1H), 7.53 (s, 1H), 7.39 (d, 1H), 6.98 (s, 1H), 4.13 (m, 2H), 3.89 (s, 3H), 3.85 (s, 3H), 3.68 (m, 2H), 3.32 (s, 3H), 2.42 (s, 3H). HR-MS (ES⁺) m/z Calculated for C₂₃H₂₄N₂NaO₇ ([M+Na]⁺): 463.1476. Found: 463.1475.

### Example 24

### 7-Methoxy-6-(2-methoxy-ethoxy)-2-oxo-1,2-dihydro-quinoline-3-carboxylic acid [2-methyl-5-(1H-tetrazol-5-yl)-phenyl]-amide

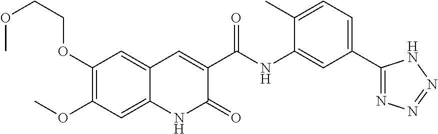

7-Methoxy-6-(2-methoxy-ethoxy)-2-oxo-1,2-dihydro-quinoline-3-carboxylic acid [2-methyl-5-(1H-tetrazol-5-yl)-phenyl]-amide was synthesized in a manner similar to Example 23 Step E with 7-methoxy-6-(2-methoxy-ethoxy)-2-oxo-1,2-dihydro-quinoline-3-carboxylic acid (0.08 g, 0.273 mmol) (from Example 23 Step D supra) and 2-methyl-5-(2H-tetrazol-5-yl)-phenylamine (0.068 g, 0.363 mmol) (CHEMBRDG BB). (Yield 0.036 g, 29%).

¹H NMR (DMSO-d₆): δ 12.46 (br s, 1H), 12.16 (br s, 1H), 8.95 (s, 1H), 8.88 (s, 1H), 7.63 (d, 1H), 7.56 (s, 1H), 7.25 (d, 1H), 6.98 (s, 1H), 4.14 (m, 2H), 3.88 (s, 3H), 3.68 (m, 2H), 3.31 (s, 3H), 2.36 (s, 3H). (One NH not detected). HR-MS (ES⁺) m/z Calculated for C₂₂H₂₃N₆O₅ ([M+H]⁺): 451.1725. Found: 451.1723.

### Example 25

### 3-{[6,7-Bis-(2-methoxy-ethoxy)-2-oxo-1,2-dihydro-quinoline-3-carbonyl]-amino}-4-methyl-benzoic acid methyl ester

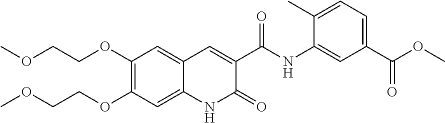

### Step A

### 3,4-Bis-(2-methoxy-ethoxy)-benzaldehyde

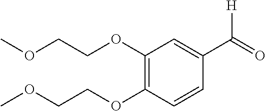

To a solution of 3,4-dihydroxybenzaldehyde (10 g, 72.5 mmol) (Aldrich) in DMF (200 mL) was added 2-chloroethyl methyl ether (16.5 mL, 181.2 mmol) (Aldrich), potassium carbonate (50 g, 362.3 mmol) and a catalytic amount of potassium iodide (Aldrich). The reaction mixture was heated at 85° C. for 14 hours. The reaction mixture was cooled down, diluted with EtOAc and washed with brine. The organic phase was dried over anhydrous sodium sulfate, evaporated to afford 3,4-bis-(2-methoxy-ethoxy)-benzaldehyde as an oil and was used in the next step without further purification. (Yield 11 g, 70%).

### Step B

### 4,5-Bis-(2-methoxy-ethoxy)-2-nitro-benzaldehyde

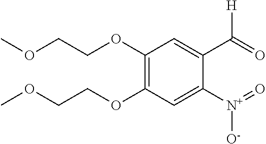

To a solution of 3,4-bis-(2-methoxy-ethoxy)-benzaldehyde (6 g, 23.6 mmol) in acetic acid (30 mL) cooled at 4° C., was added nitric acid (3 mL) (Aldrich). The reaction mixture was stirred for 1 hour. Then, sulfuric acid (3 mL) and potassium nitrate (2.62 g, 26 mmol) (Aldrich) were added to the reaction mixture which was slowly warmed to room temperature and stirred overnight. After completion of the reaction, ammonium hydroxide was added to obtain a pH=10. The reaction mixture was diluted with EtOAc and washed with brine. The combined organic phase was dried over anhydrous sodium sulfate and evaporated. The crude material was purified by column chromatography. The desired compound was eluted with 70% EtOAc. The fractions were evaporated to afford 4,5-bis-(2-methoxy-ethoxy)-2-nitro-benzaldehyde. (Yield 4.5 g, 64%).

### Step C

### 6,7-Bis-(2-methoxy-ethoxy)-2-oxo-1,2-dihydro-quinoline-3-carboxylic acid methyl ester

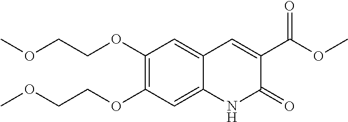

To a solution of 4,5-bis-(2-methoxy-ethoxy)-2-nitro-benzaldehyde (2.4 g, 8.03 mmol) in EtOAc (60 mL) was added Pd/C (240 mg) (Aldrich). The reaction mixture was hydrogenated using a Parr hydrogenator system at 30 PSI for 2 hours. Upon completion of the reaction, the solution was filtered over a plug of Celite then the filtrate evaporated without heating the water bath. The crude oil was then dissolved in MeOH (30 mL). Dimethyl malonate (2 mL, 16.06 mmol) (Aldrich), piperidine (2 mL, 16.06 mmol) (Aldrich) and 2 drops of acetic acid were added to the solution. The reaction mixture was heated at 65° C. for 18 hours. The reaction mixture was cooled down and the solvent was evaporated. MeOH was added to the crude paste which was filtered. The paste was washed several times with MeOH and let dried under air to afford 6,7-bis-(2-methoxy-ethoxy)-2-oxo-1,2-dihydro-quinoline-3-carboxylic acid methyl ester as a yellow solid. (Yield 0.45 g, 17%).

### Step D

### 6,7-Bis-(2-methoxy-ethoxy)-2-oxo-1,2-dihydro-quinoline-3-carboxylic acid

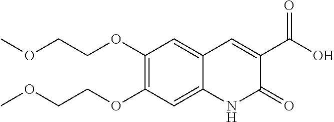

To a solution of 6,7-bis-(2-methoxy-ethoxy)-2-oxo-1,2-dihydro-quinoline-3-carboxylic acid methyl ester (0.4 g, 1.14 mmol) in MeOH (5 mL) was added a solution of NaOH (0.456 g, 11.4 mmol) in water (5 mL). The reaction mixture was heated at 60° C. for 2 hours. Once the reaction was complete, the reaction mixture was cooled down to afford a thick paste to which a 10% HCl solution was added. The resulting suspension was filtered to afford 6,7-bis-(2-methoxy-ethoxy)-2-oxo-1,2-dihydro-quinoline-3-carboxylic acid. (Yield 0.287 g, 75%).

### Step E

### 3-{[6,7-Bis-(2-methoxy-ethoxy)-2-oxo-1,2-dihydro-quinoline-3-carbonyl]-amino}-4-methyl-benzoic acid methyl ester

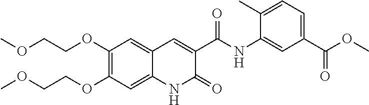

To a solution of 6,7-bis-(2-methoxy-ethoxy)-2-oxo-1,2-dihydro-quinoline-3-carboxylic acid (0.1 g, 0.30 mmol) in DMF (5 mL) was added HATU (0.137 g, 0.36 mmol) (Aldrich), triethylamine (0.1 mL, 0.72 mmol) (Fluka) and 3-amino-4-methyl-benzoic acid methyl ester (0.059 g, 0.36 mmol) (TCI). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 3-{[6,7-bis-(2-methoxy-ethoxy)-2-oxo-1,2-dihydro-quinoline-3-carbonyl]-amino}-4-methyl-benzoic acid methyl ester(1-phenyl-propyl)-amide. (Yield 0.064 g, 55%). HR-MS (ES⁺) m/z Calculated for C₂₅H₂₉N₂O₈ ([M+H]⁺): 485.1919. Found: 485.1919.

### Example 26

### 3-{[6-Methoxy-7-(2-methoxy-ethoxy)-2-oxo-1,2-dihydro-quinoline-3-carbonyl]-amino}-4-methyl-benzoic acid methyl ester

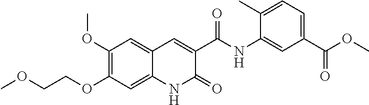

### Step A

### 3-Methoxy-4-(2-methoxy-ethoxy)-benzaldehyde

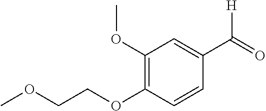

3-Methoxy-4-(2-methoxy-ethoxy)-benzaldehyde was synthesized in a manner similar to Example 23 step A with 4-hydroxy-3-methoxy-benzaldehyde (10 g, 65.72 mmol) (Aldrich) and 1-chloro-2-methoxy-ethane (6.83 g, 6.6 mL, 72.30 mmol) (Aldrich). (Yield 14.15 g, 100%).

LR-MS [M+H]⁺211.

### Step B

### 5-Methoxy-4-(2-methoxy-ethoxy)-2-nitro-benzaldehyde

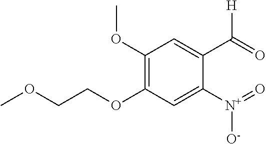

5-Methoxy-4-(2-methoxy-ethoxy)-2-nitro-benzaldehyde was synthesized in a manner similar to Example 23 step B with 3-methoxy-4-(2-methoxy-ethoxy)-benzaldehyde (5 g, 23.78 mmol), nitric acid (7.5 mL) (Aldrich) and potassium nitrate (2.35 g, 23.2 mmol) (Aldrich) and was used in next step without purification. (Yield 4.25 g, 70%).

### Step C

### 6-Methoxy-7-(2-methoxy-ethoxy)-2-oxo-1,2-dihydro-quinoline-3-carboxylic acid methyl ester

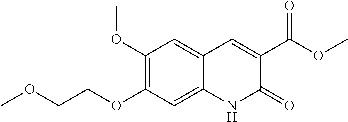

6-Methoxy-7-(2-methoxy-ethoxy)-2-oxo-1,2-dihydro-quinoline-3-carboxylic acid methyl ester was synthesized in a manner similar to Example 23 step C with 5-methoxy-4-(2-methoxy-ethoxy)-2-nitro-benzaldehyde (2.5 g, 9.80 mmol). (Yield 1.023 g, 28% for two steps).

¹H NMR (DMSO-d₆): δ 11.84 (br s, 1H), 8.45 (s, 1H), 7.35 (s, 1H), 6.81 (s, 1H), 4.11 (m, 2H), 3.77 (s, 3H), 3.75 (s, 3H), 3.69 (m, 2H), 3.30 (s, 3H).

### Step D

### 6-Methoxy-7-(2-methoxy-ethoxy)-2-oxo-1,2-dihydro-quinoline-3-carboxylic acid

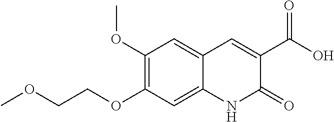

6-Methoxy-7-(2-methoxy-ethoxy)-2-oxo-1,2-dihydro-quinoline-3-carboxylic acid was synthesized in a manner similar to Example 23 step D with 6-methoxy-7-(2-methoxy-ethoxy)-2-oxo-1,2-dihydro-quinoline-3-carboxylic acid methyl ester (1 g, 3.26 mmol) and NaOH (0.261 g, 6.52 mmol). (Yield 0.911 g, 96%).

¹H NMR (DMSO-d₆): δ 8.81 (s, 1H), 7.55 (s, 1H), 6.97 (s, 1H), 4.19 (m, 2H), 3.82 (s, 3H), 3.71 (s, 3H), 3.31 (s, 3H).

### Step E

### 3-{[6-Methoxy-7-(2-methoxy-ethoxy)-2-oxo-1,2-dihydro-quinoline-3-carbonyl]-amino}-4-methyl-benzoic acid methyl ester

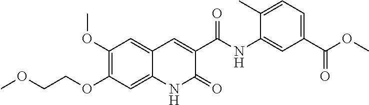

3-{[6-Methoxy-7-(2-methoxy-ethoxy)-2-oxo-1,2-dihydro-quinoline-3-carbonyl]-amino}-4-methyl-benzoic acid methyl ester was synthesized in a manner similar to Example 23 step E with 6-methoxy-7-(2-methoxy-ethoxy)-2-oxo-1,2-dihydro-quinoline-3-carboxylic acid (0.1 g, 0.34 mmol) and 3-amino-4-methyl-benzoic acid methyl ester (0.075 g, 0.45 mmol) (TCI). (Yield 0.094 g, 62%).

¹H NMR (DMSO-d₆): δ 12.46 (br s, 1H), 12.39 (br s, 1H), 9.01 (s, 1H), 8.88 (s, 1H), 7.62 (d, 1H), 7.52 (s, 1H), 7.39 (d, 1H), 6.97 (s, 1H), 4.18 (m, 2H), 3.85 (s, 3H), 3.84 (s, 3H), 3.73 (m, 2H), 3.32 (s, 3H), 2.42 (s, 3H). HR-MS (ES⁺) m/z Calculated for C₂₃H₂₅N₂O₇ ([M+H]⁺): 441.1657. Found: 441.1656.

### Example 27

### 6-Methoxy-7-(2-methoxy-ethoxy)-2-oxo-1,2-dihydro-quinoline-3-carboxylic acid [2-methyl-5-(1H-tetrazol-5-yl)-phenyl]-amide

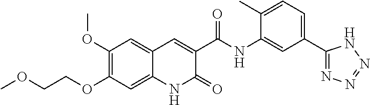

6-Methoxy-7-(2-methoxy-ethoxy)-2-oxo-1,2-dihydro-quinoline-3-carboxylic acid [2-methyl-5-(1H-tetrazol-5-yl)-phenyl]-amide was synthesized in a manner similar to Example 23 step E with 6-methoxy-7-(2-methoxy-ethoxy)-2-oxo-1,2-dihydro-quinoline-3-carboxylic acid (0.1 g, 0.34 mmol) (from Example 26 step D supra) and 2-methyl-5-(2H-tetrazol-5-yl)-phenylamine (79 mg, 0.45 mmol) (CHEMBRDG BB). (Yield 74 mg, 48%).

¹H NMR (DMSO-D6): δ 12.44 (br s, 1H), 12.21 (br s, 1H), 8.98 (s, 1H), 8.90 (s, 1H), 7.65 (d, 1H), 7.57 (s, 1H), 7.30 (d, 1H), 6.98 (s, 1H), 4.18 (m, 2H), 3.83 (s, 3H), 3.73 (m, 2H), 3.32 (s, 3H), 2.37 (s, 3H). HR-MS (ES⁺) m/z Calculated for C₂₂H₂₃N₆O₅ ([M+H]⁺): 451.1725. Found: 451.1724.

### Example 28

### 6,7-Bis-(2-methoxy-ethoxy)-2-oxo-1,2-dihydro-quinoline-3-carboxylic acid [2-methyl-5-(1H-tetrazol-5-yl)-phenyl]-amide

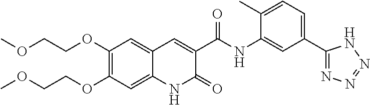

6,7-Bis-(2-methoxy-ethoxy)-2-oxo-1,2-dihydro-quinoline-3-carboxylic acid [2-methyl-5-(1H-tetrazol-5-yl)-phenyl]-amide was synthesized in a manner similar to Example 23 step E with 6,7-bis-(2-methoxy-ethoxy)-2-oxo-1,2-dihydro-quinoline-3-carboxylic acid (0.1 g, 0.297 mmol) (from Example 25 step D supra) and 2-methyl-5-(2H-tetrazol-5-yl)-phenylamine (0.069 g, 0.395 mmol) (CHEMBRDG BB). (Yield 0.116 g, 79%).

¹H NMR (DMSO-d₆): δ 12.43 (br s, 1H), 12.17 (br s, 1H), 8.95 (s, 1H), 8.88 (s, 1H), 7.65 (d, 1H), 7.58 (s, 1H), 7.27 (d, 1H), 6.99 (s, 1H), 4.21-4.15 (m, 4H), 3.75-3.67 (m, 4H), 3.35-3.32 (m, 6H), 2.36 (s, 3H). HR-MS (ES⁺) m/z Calculated for C₂₄H₂₇N₆O₆ ([M+H]⁺): 495.1987. Found: 495.1986.

### Example 29

rac-(2-{3-[(6,7-Dimethoxy-2-oxo-1,2-dihydro-quinoline-3-carbonyl)-amino]-4-methyl-benzoylamino}-2-phenyl-ethyl)-carbamic acid tert-butyl ester

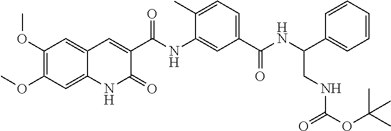

To a solution of 3-[(6,7-dimethoxy-2-oxo-1,2-dihydro-quinoline-3-carbonyl)-amino]-4-methyl-benzoic acid (0.13 g, 0.34 mmol) (from Example 18 supra) in DMF (5 mL) was added HATU (0.167 g, 0.44 mmol) (Aldrich), triethylamine (0.1 mL) (Aldrich) and (2-amino-2-phenyl-ethyl)-carbamic acid tert-butyl ester (0.104 g, 0.44 mmol) (from Example 1 supra). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide rac-(2-{3-[(6,7-dimethoxy-2-oxo-1,2-dihydro-quinoline-3-carbonyl)-amino]-4-methyl-benzoylamino}-2-phenyl-ethyl)-carbamic acid tert-butyl ester. (Yield 0.148 g, 72%). HR-MS (ES⁺) m/z Calculated for C₃₃H₃₇N₄O₇ ([M+H]⁺): 601.2657. Found: 601.2657.

### Example 30

### (2-{3-[(6,7-Dimethoxy-2-oxo-1,2-dihydro-quinoline-3-carbonyl)-amino]-4-methyl-benzoylamino}-ethyl)-carbamic acid tert-butyl ester

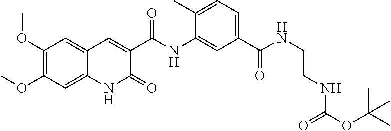

To a solution of 3-[(6,7-dimethoxy-2-oxo-1,2-dihydro-quinoline-3-carbonyl)-amino]-4-methyl-benzoic acid (0.13 g, 0.34 mmol) (from Example 18 supra) in DMF (5 mL) was added HATU (0.167 g, 0.44 mmol) (Aldrich), triethylamine (0.1 mL) (Aldrich) and (2-amino-ethyl)-carbamic acid tert-butyl ester (0.07 mL 0.44 mmol) (Aldrich). The reaction mixture was stirred at room temperature for 18 hours. Then, water (10 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide (2-{3-[(6,7-dimethoxy-2-oxo-1,2-dihydro-quinoline-3-carbonyl)-amino]-4-methyl-benzoylamino}-ethyl)-carbamic acid tert-butyl ester. (Yield 0.046 g, 26%). HR-MS (ES⁺) m/z Calculated for C₂₇H₃₂N₄NaO₇ ([M+Na]⁺): 547.2163. Found: 547.2165.

### Example 31

### 6,7-Dimethoxy-2-oxo-1,2-dihydro-quinoline-3-carboxylic acid [5-(2-amino-1-phenyl-ethylcarbamoyl)-2-methyl-phenyl]-amide

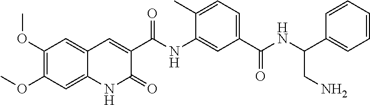

(2-{3-[(6,7-Dimethoxy-2-oxo-1,2-dihydro-quinoline-3-carbonyl)-amino]-4-methyl-benzoylamino}-2-phenyl-ethyl)-carbamic acid tert-butyl ester (0.05 g, 0.08 mmol) was dissolved in dichloromethane (2 mL) and TFA (2 mL). The reaction mixture was stirred at room temperature for 3 hours, then the solvent was evaporated. A saturated aqueous sodium bicarbonate solution and EtOAc were added and the suspension was filtered and dried under air overnight to afford 6,7-dimethoxy-2-oxo-1,2-dihydro-quinoline-3-carboxylic acid [5-(2-amino-1-phenyl-ethylcarbamoyl)-2-methyl-phenyl]-amide as a yellow solid. (Yield 0.025 g, 63%). HR-MS (ES⁺) m/z Calculated for C₂₈H₂₉N₄O₅ ([M+H]⁺): 501.2133. Found: 501.2131.

### Example 32

### 6,7-Dimethoxy-2-oxo-1,2-dihydro-quinoline-3-carboxylic acid [2-methyl-5-(3-morpholin-4-yl-propylcarbamoyl)-phenyl]-amide

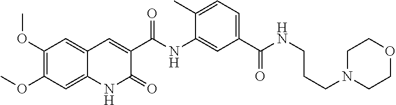

To a solution of 3-[(6,7-dimethoxy-2-oxo-1,2-dihydro-quinoline-3-carbonyl)-amino]-4-methyl-benzoic acid (0.1 g, 0.26 mmol) (from Example 18 supra) in DMF (5 mL) was added HATU (0.128 g, 0.34 mmol) (Aldrich), triethylamine (0.1 mL) (Aldrich) and N-(3-aminopropyl)-morpholine (0.05 mL 0.34 mmol) (Aldrich). The reaction mixture was stirred at room temperature for 18 hours. Then, water (10 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid [2-methyl-5-(3-morpholin-4-yl-propylcarbamoyl)-phenyl]-amide. (Yield 0.022 g, 17%).

HR-MS (ES⁺) m/z Calculated for C₂₇H₃₂N₄O₆ ([M+H]⁺): 509.2395. Found: 509.2392.

### Example 33

### 6,7-Dimethoxy-2-oxo-1,2-dihydro-quinoline-3-carboxylic acid [5-(1-hydroxymethyl-2-methyl-propylcarbamoyl)-2-methyl-phenyl]-amide

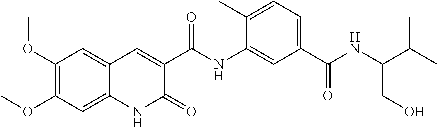

To a solution of 3-[(6,7-dimethoxy-2-oxo-1,2-dihydro-quinoline-3-carbonyl)-amino]-4-methyl-benzoic acid (0.1 g, 0.26 mmol) (from Example 18 supra) in DMF (5 mL) was added HATU (0.128 g, 0.34 mmol) (Aldrich), triethylamine (0.1 mL) (Aldrich) and 2-amino-3-methyl-butan-1-ol (0.04 mL, 0.34 mmol) (Aldrich). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid [5-(1-hydroxymethyl-2-methyl-propylcarbamoyl)-2-methyl-phenyl]-amide. (Yield 0.03 g, 25%).

HR-MS (ES⁺) m/z Calculated for C₂₅H₃₀N₃O₆ ([M+H]⁺): 468.2129. Found: 468.2128.

### Example 34

### (3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester

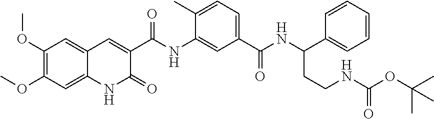

To a solution of 3-[(6,7-dimethoxy-2-oxo-1,2-dihydro-quinoline-3-carbonyl)-amino]-4-methyl-benzoic acid (0.3 g, 0.79 mmol) (from Example 18 supra) in DMF (10 mL) was added HATU (0.39 g, 0.1.03 mmol) (Aldrich), triethylamine (0.2 mL) (Aldrich) and (3-amino-3-phenyl-propyl)-carbamic acid tert-butyl ester (0.257 g, 1.03 mmol) (from Example 3 supra). The reaction mixture was stirred at room temperature for 18 hours. Then, water (10 mL), saturated aqueous sodium bicarbonate solution (10 mL) and ethyl acetate (10 mL) were added. After mixing, the precipitate was filtered under vacuum and washed with ethyl acetate. The resulting solid was dried under air to provide (3-{3-[(6,7-dimethoxy-2-oxo-1,2-dihydro-quinoline-3-carbonyl)-amino]-4-methyl-benzoylamino}-3-phenyl-propyl)-carbamic acid tert-butyl ester. (Yield 0.333 g, 69%). HR-MS (ES⁺) m/z Calculated for C₃₄H₃₈N₄NaO₇ ([M+Na]⁺): 637.2632. Found: 637.2631.

### Example 35

### 6,7-Dimethoxy-2-oxo-1,2-dihydro-quinoline-3-carboxylic acid [5-(3-amino-1-phenyl-propylcarbamoyl)-2-methyl-phenyl]-amide

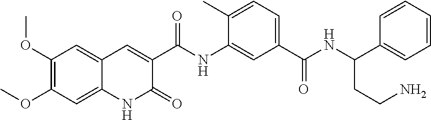

(3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester (0.09 g, 0.15 mmol) (from Example 34 supra) was dissolved in dichloromethane (2 mL) and TFA (2 mL). The reaction mixture was stirred at room temperature for 2 hours, then the solvent was evaporated. A saturated aqueous sodium bicarbonate solution and EtOAc were added and the suspension was filtered and dried under air overnight to provide 6,7-dimethoxy-2-oxo-1,2-dihydro-quinoline-3-carboxylic acid [5-(3-amino-1-phenyl-propylcarbamoyl)-2-methyl-phenyl]amide as a yellow solid. (Yield 0.02 g, 26%).

HR-MS (ES⁺) m/z Calculated for C₂₉H₃₀N₄O₅ ([M+H]⁺): 515.2289. Found: 515.2285.

### Example 36

### 6,7-Dimethoxy-2-oxo-1,2-dihydro-quinoline-3-carboxylic acid (3-chloro-pyridin-4-yl)-amide

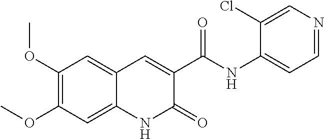

6,7-Dimethoxy-2-oxo-1,2-dihydro-quinoline-3-carboxylic acid (3-chloro-pyridin-4-yl)-amide was synthesized in a manner similar to Example 23 step E with 6,7-dimethoxy-2-oxo-1,2-dihydro-quinoline-3-carboxylic acid (0.1 g, 0.403 mmol) (from Example 15 supra) and 3-chloro-pyridin-4-ylamine (0.069 g, 0.536 mmol) (Matrix). (Yield 0.038 g, 26%).

¹H NMR (DMSO-d₆): δ 8.91 (s, 1H), 8.62 (s, 1H), 8.56 (d, 1H), 8.44 (d, 1H), 7.54 (s, 1H), 6.97 (s, 1H), 3.88 (s, 3H), 3.82 (s, 3H). (Two NH not detected). HR-MS (ES⁺) m/z Calculated for C₁₇H₁₅ClN₃O₄ ([M+H]⁺): 360.0746. Found: 360.0745.

### Example 37

### 7-Methoxy-6-(2-methoxy-ethoxy)-2-oxo-1,2-dihydro-quinoline-3-carboxylic acid [5-(2-amino-1-phenyl-ethylcarbamoyl)-2-methyl-phenyl]-amide

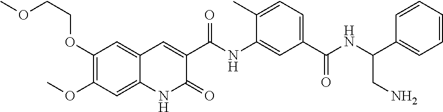

### Step A

### 3-{[7-Methoxy-6-(2-methoxy-ethoxy)-2-oxo-1,2-dihydro-quinoline-3-carbonyl]-amino}-4-methyl-benzoic acid

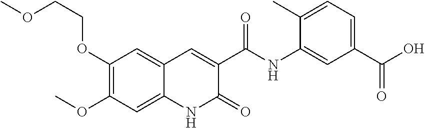

To a suspension of 3-{[7-methoxy-6-(2-methoxy-ethoxy)-2-oxo-1,2-dihydro-quinoline-3-carbonyl]-amino}-4-methyl-benzoic acid methyl ester (0.6 g, 1.36 mmol) (from Example 23 supra) in MeOH (30 mL), NaOH (0.218 g, 5.45 mmol) and H₂O (3 mL) were added and mixture heated at 60° C. for 1.5 hours. After cooling, the pH was adjusted to 3 by addition of conc. HCl (J. T. Baker). Precipitate was formed, and was filtered, washed with water (2×10 mL), MeOH (1×5 mL) and dried under reduced pressure overnight to give 3-{[7-methoxy-6-(2-methoxy-ethoxy)-2-oxo-1,2-dihydro-quinoline-3-carbonyl]-amino}-4-methyl-benzoic acid as a yellow solid. (Yield 0.314 g, 54%).

¹H NMR (DMSO-d₆): δ 12.47 (br s, 1H), 12.31 (br s, 1H), 8.96 (s, 1H), 8.86 (s, 1H), 7.61 (d, 1H), 7.54 (s, 1H), 7.35 (d, 1H), 6.98 (s, 1H), 4.14 (m, 2H), 3.89 (s, 3H), 3.69 (m, 2H), 3.32 (s, 3H), 2.40 (s, 3H). (One H not detected).

### Step B

### 7-Methoxy-6-(2-methoxy-ethoxy)-2-oxo-1,2-dihydro-quinoline-3-carboxylic acid [5-(2-amino-1-phenyl-ethylcarbamoyl)-2-methyl-phenyl]-amide

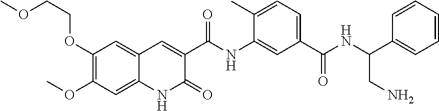

To a suspension of 3-{[7-methoxy-6-(2-methoxy-ethoxy)-2-oxo-1,2-dihydro-quinoline-3-carbonyl]-amino}-4-methyl-benzoic acid (0.1 g, 0.235 mmol) in DMF (4 mL), HATU (0.107 g, 0.282 mmol) (Aldrich) was added, followed by DIPEA (0.036 g, 0.05 mL, 0.282 mmol) (Aldrich). The mixture was stirred for 5 mins. The reaction turned into a clear solution. tert-Butyl 2-amino-2-phenylethylcarbamate (0.074 g, 0.312 mmol) (from Example 1 supra) was added and mixture stirred at room temperature overnight. The reaction was diluted with EtOAc (150 mL), washed with water (50 mL), brine, and dried with anhydrous sodium sulfate. Concentration gave an oil, which was used in next step without further purification. This oil was dissolved in dichloromethane (6 mL), TFA (3 mL) (Aldrich) was added and mixture stirred at room temperature for 3 hours. The solvent was removed under reduced pressure. The residue was dissolved in MeOH (6 mL), neutralized with sat. NaHCO₃ solution, and purified by preparative HPLC to provide 7-methoxy-6-(2-methoxy-ethoxy)-2-oxo-1,2-dihydro-quinoline-3-carboxylic acid [5-(2-amino-1-phenyl-ethylcarbamoyl)-2-methyl-phenyl]amide as a yellow solid. (Yield 0.06 g, 48% in two steps). HR-MS (ES⁺) m/z Calculated for C₃₀H₃₂N₄O₆ ([M+H]⁺): 545.2395. Found: 545.2394.

### Example 38

### 7-Methoxy-6-(2-methoxy-ethoxy)-2-oxo-1,2-dihydro-quinoline-3-carboxylic acid [5-(3-amino-1-phenyl-propylcarbamoyl)-2-methyl-phenyl]-amide

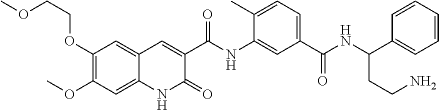

7-Methoxy-6-(2-methoxy-ethoxy)-2-oxo-1,2-dihydro-quinoline-3-carboxylic acid [5-(3-amino-1-phenyl-propylcarbamoyl)-2-methyl-phenyl]-amide was synthesized in a manner similar to Example 37 with 3-{[7-methoxy-6-(2-methoxy-ethoxy)-2-oxo-1,2-dihydro-quinoline-3-carbonyl]-amino}-4-methyl-benzoic acid (0.1 g, 0.235 mmol) (from Example 37 step A supra) and (3-amino-3-phenyl-propyl)-carbamic acid tert-butyl ester (0.078 g, 0.312 mmol) (from Example 3 supra). (Yield 0.073 g, 39%).

¹H NMR (DMSO-d₆): δ 12.23 (br s, 1H), 8.93 (m, 1H), 8.85 (s, 1H), 8.75 (s, 1H), 7.53-7.55 (m, 2H), 7.41-7.20 (m, 6H), 6.98 (s, 1H), 5.14 (m, 1H), 4.14 (m, 2H), 3.88 (s, 3H), 3.68 (m, 2H), 3.30 (s, 3H), 2.66 (m, 2H), 2.38 (s, 3H), 1.93 (m, 2H). (Three NH not detected). HR-MS (ES⁺) m/z Calculated for C₃₁H₃₅N₄O₆ ([M+H]⁺): 559.2551. Found: 559.2547.

### Example 39

### 3-{[7-Methoxy-6-(2-morpholin-4-yl-ethoxy)-2-oxo-1,2-dihydro-quinoline-3-carbonyl]-amino}-4-methyl-benzoic acid methyl ester

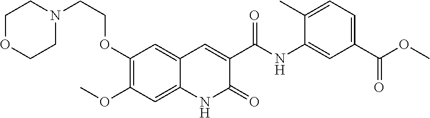

### Step A

### 4-Methoxy-3-(2-morpholin-4-yl-ethoxy)-benzaldehyde

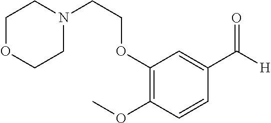

4-Methoxy-3-(2-morpholin-4-yl-ethoxy)-benzaldehyde was synthesized in a manner similar to Example 23 step A with 3-hydroxy-4-methoxy-benzaldehyde (10 g, 65.73 mmol) (Aldrich) and 4-(2-chloro-ethyl)-morpholine (13.45 g, 72.30 mmol) (Princeton). (Yield 15.6 g, 78%). ¹H NMR (DMSO-d₆): δ 10.17 (s, 1H), 7.90 (m, 1H), 7.78 (m, 1H), 7.16 (d, 1H), 4.17 (t, 2H), 3.89 (s, 3H), 3.55 (m, 4H), 2.70 (m, 2H), 2.48 (m, 4H).

### Step B

### 4-Methoxy-5-(2-morpholin-4-yl-ethoxy)-2-nitro-benzaldehyde

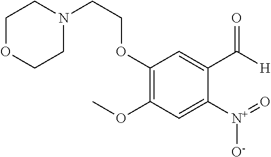

4-Methoxy-5-(2-morpholin-4-yl-ethoxy)-2-nitro-benzaldehyde was synthesized in a manner similar to Example 23 step B with 4-methoxy-3-(2-morpholin-4-yl-ethoxy)-benzaldehyde (5 g, 18.86 mmol), nitric acid (7.5 mL) (Aldrich) and potassium nitrate (2.35 g, 23.2 mmol) (Aldrich). (Yield 4.60 g, 78%).

¹H NMR (DMSO-d₆): δ 10.17 (s, 1H), 7.68 (s, 1H), 7.41 (s, 1H), 4.26 (t, 2H), 3.94 (s, 3H), 3.55 (m, 4H), 2.70 (m, 2H), 2.48 (m, 4H).

### Step C

### 7-Methoxy-6-(2-morpholin-4-yl-ethoxy)-2-oxo-1,2-dihydro-quinoline-3-carboxylic acid methyl ester

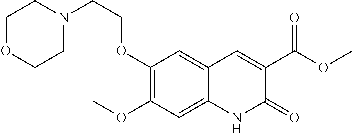

7-Methoxy-6-(2-morpholin-4-yl-ethoxy)-2-oxo-1,2-dihydro-quinoline-3-carboxylic acid methyl ester was synthesized in a manner similar to Example 23 step C with 4-methoxy-5-(2-morpholin-4-yl-ethoxy)-2-nitro-benzaldehyde (3.04 g, 9.80 mmol). (Yield 0.814 g, 18% for two steps). ¹H NMR (CDCl₃): δ 9.85 (s, 1H), 8.51 (s, 1H), 7.01 (s, 1H), 4.15 (m, 2H), 3.94 (s, 3H), 3.72 (m, 4H), 3.49 (s, 3H), 2.87 (m, 2H), 2.61 (m, 4H). (One NH not detected)

### Step D

### 7-Methoxy-6-(2-morpholin-4-yl-ethoxy)-2-oxo-1,2-dihydro-quinoline-3-carboxylic acid

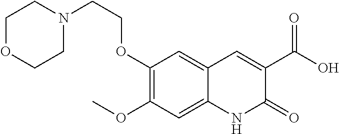

To a solution of 7-methoxy-6-(2-morpholin-4-yl-ethoxy)-2-oxo-1,2-dihydro-quinoline-3-carboxylic acid methyl ester (0.373 g, 1.03 mmol) in MeOH (10 mL) was added NaOH (0.150 g, 3.75 mmol) and H₂O (2 mL). The mixture was heated at 60° C. for 2 hours. The mixture was acidified to pH=3 by addition of conc. HCl (J. T. Baker). The solvent was removed under reduced pressure. The residue was purified by preparative HPLC to give 7-methoxy-6-(2-morpholin-4-yl-ethoxy)-2-oxo-1,2-dihydro-quinoline-3-carboxylic acid a yellow solid. (Yield 0.162 g, 45%).

LR-MS [M+H]⁺349.

### Step E

### 3-{[7-Methoxy-6-(2-morpholin-4-yl-ethoxy)-2-oxo-1,2-dihydro-quinoline-3-carbonyl]-amino}-4-methyl-benzoic acid methyl ester

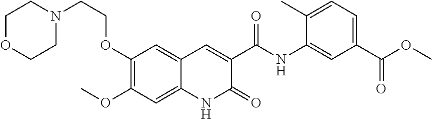

3-{[7-Methoxy-6-(2-morpholin-4-yl-ethoxy)-2-oxo-1,2-dihydro-quinoline-3-carbonyl]-amino}-4-methyl-benzoic acid methyl ester was synthesized in a manner similar to Example 23 step E with 7-methoxy-6-(2-morpholin-4-yl-ethoxy)-2-oxo-1,2-dihydro-quinoline-3-carboxylic acid (0.162 g, 0.466 mmol) and 3-amino-4-methyl-benzoic acid methyl ester (0.115 g, 0.698 mmol) (TCI). (Yield 0.106 g, 46%).

¹H NMR (DMSO-d₆): δ 12.39 (br s, 1H), 9.1 (s, 1H), 8.83 (s, 1H), 7.60 (m, 1H), 7.52 (s, 1H), 7.37 (d, 1H), 6.96 (s, 1H), 4.13 (m, 2H), 3.87 (s, 3H), 3.84 (s, 3H), 3.57 (m, 4H), 2.69 (m, 2H), 2.58 (m, 4H), 2.41 (3H). (One NH not detected). HR-MS (ES⁺) m/z Calculated for C₂₆H₃₀N₃O₇ ([M+H]⁺): 496.2079. Found: 496.2076.

### Example 40

### 6,7-Dimethoxy-2-oxo-1,2-dihydro-quinoline-3-carboxylic acid [5-(3-chloro-benzylcarbamoyl)-2-methyl-phenyl]-amide

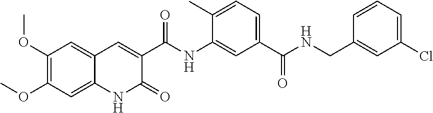

To a solution of 3-[(6,7-dimethoxy-2-oxo-1,2-dihydro-quinoline-3-carbonyl)-amino]-4-methyl-benzoic acid (0.1 g, 0.26 mmol) (from Example 18 supra) in DMF (5 mL) was added HATU (0.128 g, 0.34 mmol) (Aldrich), triethylamine (0.1 mL) (Aldrich) and 3-chlorobenzylamine (0.041 mL, 0.34 mmol) (Aldrich). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid [5-(3-chloro-benzylcarbamoyl)-2-methyl-phenyl]-amide. (Yield 0.05 g, 38%).

HR-MS (ES⁺) m/z Calculated for C₂₇H₂₅ClN₃O₅ ([M+H]⁺): 506.1477. Found: 506.1473.

### Example 41

### 6,7-Dimethoxy-2-oxo-1,2-dihydro-quinoline-3-carboxylic acid [5-(cyclohexylmethyl-carbamoyl)-2-methyl-phenyl]-amide

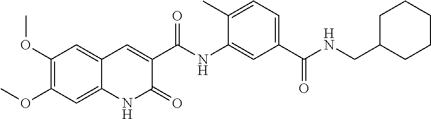

To a solution of 3-[(6,7-Dimethoxy-2-oxo-1,2-dihydro-quinoline-3-carbonyl)-amino]-4-methyl-benzoic acid (0.1 g, 0.26 mmol) (from Example 18 supra) in DMF (5 mL) was added HATU (0.128 g, 0.34 mmol) (Aldrich), triethylamine (0.1 mL) (Aldrich) and cyclohexane methyl amine (0.044 mL, 0.34 mmol) (Aldrich). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid [5-(cyclohexylmethyl-carbamoyl)-2-methyl-phenyl]-amide. (Yield 0.073 g, 60%).

HR-MS (ES⁺) m/z Calculated for C₂₇H₃₂N₃O₅ ([M+H]⁺): 478.2237. Found: 478.2235.

### Example 42

### 6,7-Dimethoxy-2-oxo-1,2-dihydro-quinoline-3-carboxylic acid (5-benzylcarbamoyl-2-methyl-phenyl)-amide

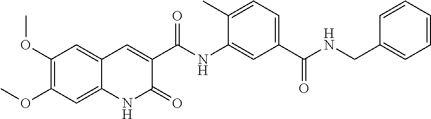

To a solution of 3-[(6,7-dimethoxy-2-oxo-1,2-dihydro-quinoline-3-carbonyl)-amino]-4-methyl-benzoic acid (0.1 g, 0.26 mmol) (from Example 18 supra) in DMF (5 mL) was added HATU (0.128 g, 0.34 mmol) (Aldrich), triethylamine (0.1 mL) (Aldrich) and benzyl amine (0.037 mL, 0.34 mmol) (Aldrich). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid (5-benzylcarbamoyl-2-methyl-phenyl)-amide. (Yield 0.088 g, 72%). HR-MS (ES⁺) m/z Calculated for C₂₇H₂₆N₃O₅ ([M+H]⁺): 472.1867. Found: 472.1866.

### Example 43

### 7-Methoxy-6-(2-morpholin-4-yl-ethoxy)-2-oxo-1,2-dihydro-quinoline-3-carboxylic acid [5-(3-chloro-benzylcarbamoyl)-2-methyl-phenyl]-amide

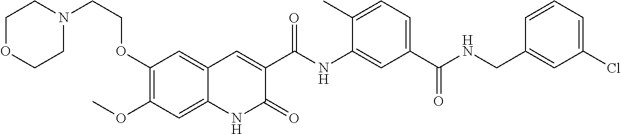

### Step A

### 3-{[7-Methoxy-6-(2-morpholin-4-yl-ethoxy)-2-oxo-1,2-dihydro-quinoline-3-carbonyl]-amino}-4-methyl-benzoic acid

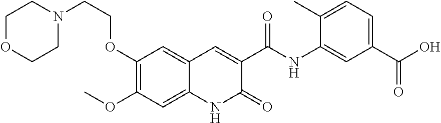

3-{[7-Methoxy-6-(2-morpholin-4-yl-ethoxy)-2-oxo-1,2-dihydro-quinoline-3-carbonyl]-amino}-4-methyl-benzoic acid was synthesized in a manner similar to Example 23 step D with 3-{[7-methoxy-6-(2-morpholin-4-yl-ethoxy)-2-oxo-1,2-dihydro-quinoline-3-carbonyl]-amino}-4-methyl-benzoic acid methyl ester (0.1 g, 0.2 mmol) (from Example 39 supra). (Yield 0.060 g, 62%).

¹H NMR (DMSO-d₆): δ 12.3 (br s, 1H), 8.96 (s, 1H), 8.88 (s, 1H), 7.67 (s, 1H), 7.59 (d, 1H), 7.36 (d, 1H), 7.03 (s, 1H), 4.45 (m, 2H), 3.97 (m, 2H), 3.90 (s, 3H), 3.76 (m, 2H), 3.59 (m, 4H), 3.24 (m, 2H), 2.41 (s, 3H). (Two NH not detected)

### Step B

### 7-Methoxy-6-(2-morpholin-4-yl-ethoxy)-2-oxo-1,2-dihydro-quinoline-3-carboxylic acid [5-(3-chloro-benzylcarbamoyl)-2-methyl-phenyl]-amide

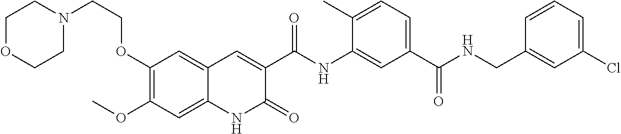

In a 10 mL round-bottomed flask, 3-{[7-methoxy-6-(2-morpholin-4-yl-ethoxy)-2-oxo-1,2-dihydro-quinoline-3-carbonyl]-amino}-4-methyl-benzoic acid (0.028 g, 0.06 mmol) was combined with DMF (2 mL) to give a yellow solution. HATU (0.034 g, 0.09 mmol) (Aldrich) and DIPEA (38 mg, 0.3 mmol) (Aldrich) were added and mixture stirred for 5 mins. 3-Chlorobenzylamine (0.013 mg, 0.09 mmol) (Aldrich) was added and mixture stirred at room temperature overnight. Mixture was purified by preparative HPLC to provide 7-methoxy-6-(2-morpholin-4-yl-ethoxy)-2-oxo-1,2-dihydro-quinoline-3-carboxylic acid [5-(3-chloro-benzylcarbamoyl)-2-methyl-phenyl]-amide as a light yellow solid. (Yield 0.026 g, 74%).

¹H NMR (DMSO-d₆): δ 12.36 (br s, 1H), 9.03 (m, 1H), 8.84 (s, 1H), 8.81 (s, 1H), 7.53 (m, 2H), 7.38-7.26 (m, 5H), 6.96 (s, 1H), 4.45 (d, 2H), 4.13 (m, 2H), 3.87 (s, 3H), 3.57 (m, 4H), 2.72 (m, 2H), 2.48 (m, 4H), 2.39 (3H). HR-MS (ES⁺) m/z Calculated for C₃₂H₃₄ClN₄O₆ ([M+H]⁺): 605.2162. Found: 605.2164.

### Example 44

### 7-Methoxy-6-(2-morpholin-4-yl-ethoxy)-2-oxo-1,2-dihydro-quinoline-3-carboxylic acid [5-(3-amino-1-phenyl-propylcarbamoyl)-2-methyl-phenyl]-amide

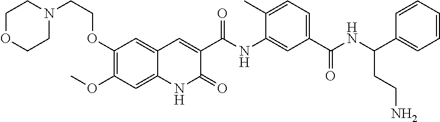

7-Methoxy-6-(2-morpholin-4-yl-ethoxy)-2-oxo-1,2-dihydro-quinoline-3-carboxylic acid [5-(3-amino-1-phenyl-propylcarbamoyl)-2-methyl-phenyl]amide was synthesized in a manner similar to Example 37 step B with 3-{[7-methoxy-6-(2-morpholin-4-yl-ethoxy)-2-oxo-1,2-dihydro-quinoline-3-carbonyl]-amino}-4-methyl-benzoic acid (0.030 g, 0.062 mmol) (from Example 43 step A supra) and (3-amino-3-phenyl-propyl)-carbamic acid tert-butyl ester (0.023 g, 0.094 mmol) (from Example 3 supra). (Yield 0.017 g, 44% for two steps). HR-MS (ES⁺) m/z Calculated for C₃₄H₄₀N₅O₆ ([M+H]⁺): 614.2973. Found: 614.2971.

### Example 45

### ((R)-3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester

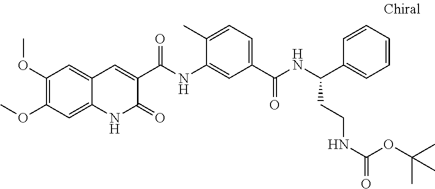

(3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester (from Example 34 supra) was separated by supercritical fluid chromatography using Kromasil OD 5-Cellucoat column with 45% MeOH in carbon dioxide as solvent. The first eluted peak (R_{T}: 6.76 min) gave ((R)-3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester.

HR-MS (ES⁺) m/z Calculated for C₃₄H₃₈N₄NaO₇ ([M+Na]⁺): 637.2632. Found: 637.2635.

### Example 46

### ((S)-3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester

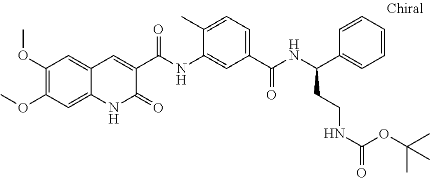

(3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester (from Example 34 supra) was separated by supercritical fluid chromatography (from Example 45 supra). The second eluted peak (R_{T}: 8.18 min) gave ((S)-3-{3-[(6,7-dimethoxy-2-oxo-1,2-dihydro-quinoline-3-carbonyl)-amino]-4-methyl-benzoylamino}-3-phenyl-propyl)-carbamic acid tert-butyl ester. HR-MS (ES⁺) m/z Calculated for C₃₄H₃₈N₄NaO₇ ([M+Na]⁺): 637.2632. Found: 637.2633.

### Example 47

### 6,7-Dimethoxy-2-oxo-1,2-dihydro-quinoline-3-carboxylic acid [5-((R)-3-amino-1-phenyl-propylcarbamoyl)-2-methyl-phenyl]-amide

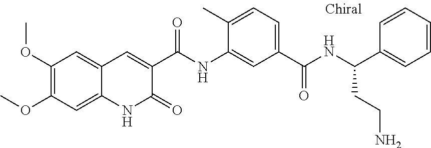

((R)-3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester (0.085 g, 0.138 mmol) (from Example 45 supra) was dissolved in dichloromethane (2 mL) and TFA (2 mL). The reaction mixture was stirred at room temperature for 2 hours, then the solvent was evaporated. A 2M NaOH solution and EtOAc were added and the suspension was filtered and dried under air overnight to provide 6,7-dimethoxy-2-oxo-1,2-dihydro-quinoline-3-carboxylic acid [5-((R)-3-amino-1-phenyl-propylcarbamoyl)-2-methyl-phenyl]-amide. (Yield 0.060 g, 86%).

HR-MS (ES⁺) m/z Calculated for C₂₉H₃₁N₄O₅ ([M+H]⁺): 515.2289. Found: 515.2292.

### Example 48

### 6,7-Dimethoxy-2-oxo-1,2-dihydro-quinoline-3-carboxylic acid [5-(S)-3-amino-1-phenyl-propylcarbamoyl)-2-methyl-phenyl]-amide

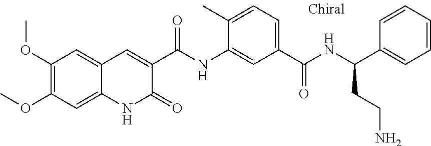

((S)-3-{3-[(6,7-Dimethoxy-2-oxo-1,2-dihydro-quinoline-3-carbonyl)-amino]-4-methyl-benzoylamino}-3-phenyl-propyl)-carbamic acid tert-butyl ester (0.090 g, 0.146 mmol) (from Example 46 supra) was dissolved in dichloromethane (2 mL) and TFA (2 mL). The reaction mixture was stirred at room temperature for 2 hours, then the solvent was evaporated. 2M NaOH solution and EtOAc were added and the suspension was filtered and dried under air overnight to provide 6,7-dimethoxy-2-oxo-1,2-dihydro-quinoline-3-carboxylic acid [5-(S)-3-amino-1-phenyl-propylcarbamoyl)-2-methyl-phenyl]-amide. (Yield 0.060 g, 80%). HR-MS (ES⁺) m/z Calculated for C₂₉H₃₁N₄O₅ ([M+H]⁺): 515.2289. Found: 515.2286.

### Example 49

### (2-(3-Chloro-phenyl)-2-{3-[(6,7-dimethoxy-2-oxo-1,2-dihydro-quinoline-3-carbonyl)-amino]-4-methyl-benzoylamino}-ethyl)-carbamic acid tert-butyl ester

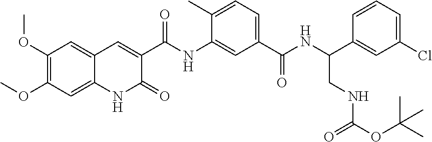

To a solution of 3-[(6,7-dimethoxy-2-oxo-1,2-dihydro-quinoline-3-carbonyl)-amino]-4-methyl-benzoic acid (0.2 g, 0.52 mmol) (from Example 18 supra) in DMF (7 mL) was added HATU (0.258 g, 0.68 mmol) (Aldrich), triethylamine (0.2 mL) (Aldrich) and [2-amino-2-(3-chloro-phenyl)-ethyl]-carbamic acid tert-butyl ester (0.182 g, 0.68 mmol) (from Example 8 supra). The reaction mixture was stirred at room temperature for 18 hours. Then, water (10 mL), saturated aqueous sodium bicarbonate solution (10 mL) and ethyl acetate (10 mL) were added. After mixing, the precipitate was filtered under vacuum and washed with ethyl acetate. The resulting solid was dried under air to provide (2-(3-chloro-phenyl)-2-{3-[(6,7-dimethoxy-2-oxo-1,2-dihydro-quinoline-3-carbonyl)-amino]-4-methyl-benzoylamino}-ethyl)-carbamic acid tert-butyl ester. (Yield 0.173 g, 52%). HR-MS (ES⁺) m/z Calculated for C₃₃H₃₅ClN₄NaO₇ ([M+Na]⁺): 657.2086. Found: 657.2088.

### Example 50

### 6,7-Dimethoxy-2-oxo-1,2-dihydro-quinoline-3-carboxylic acid {5-[2-amino-1-(3-chloro-phenyl)-ethylcarbamoyl]-2-methyl-phenyl}-amide

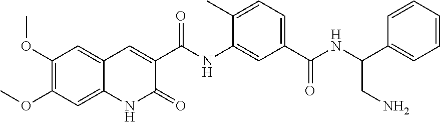

(2-(3-Chloro-phenyl)-2-{3-[(6,7-dimethoxy-2-oxo-1,2-dihydro-quinoline-3-carbonyl)-amino]-4-methyl-benzoylamino}-ethyl)-carbamic acid tert-butyl ester (0.17 g, 0.27 mmol) (from Example 49 supra) was dissolved in dichloromethane (2 mL) and TFA (2 mL). The reaction mixture was stirred at room temperature for 2 hours, then the solvent was evaporated. 2M NaOH solution and EtOAc were added and the solution was filtered and dried under air overnight to provide 6,7-dimethoxy-2-oxo-1,2-dihydro-quinoline-3-carboxylic acid {5-[2-amino-1-(3-chloro-phenyl)-ethylcarbamoyl]-2-methyl-phenyl}-amide. (Yield 0.11 g, 77%). HR-MS (ES⁺) m/z Calculated for C₂₈H₂₈ClN₄O₅ ([M+H]⁺): 535.1743. Found: 535.1745.

### Example 51

### 6,7-Dimethoxy-2-oxo-1,2-dihydro-quinoline-3-carboxylic acid (2-methyl-5-oxazol-2-yl-phenyl)-amide

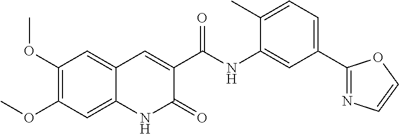

### Step A

### 2-Methyl-5-oxazol-2-yl-phenylamine

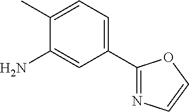

To a solution of 5-iodo-2-methyl-phenylamine (0.5 g, 1.62 mmol) (Aldrich) in toluene (5 mL) were added 2-(tri-n-butylstannyl-oxazole (0.5 mL, 2.43 mmol) (Aldrich) and tetrakis-(triphenyl-phosphine)palladium(0) (0.018 g, 0.01 mmol) (Strem Chemicals). The reaction mixture was heated at 100° C. for 14 hours. The reaction mixture was cooled down and the solvent was evaporated. The crude material was then purified by column chromatography. The desired product was eluted with 40% EtOAc. The desired fractions were evaporated to afford 2-methyl-5-oxazol-2-yl-phenylamine. (Yield 0.03 g, 11%).

HR-MS (ES⁺) m/z Calculated for C₁₀H₁₁N₂O ([M+H]⁺): 175.0866. Found: 175.0865.

### Step B

### 6,7-Dimethoxy-2-oxo-1,2-dihydro-quinoline-3-carboxylic acid (2-methyl-5-oxazol-2-yl-phenyl)-amide

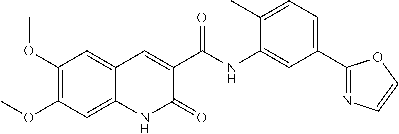

To a solution of 6,7-dimethoxy-2-oxo-1,2-dihydro-quinoline-3-carboxylic acid (0.021 g, 0.08 mmol) (from Example 15 supra) in DMF (3 mL) was added HATU (0.042 g, 0.11 mmol) (Aldrich), triethylamine (0.050 mL) and 2-methyl-5-oxazol-2-yl-phenylamine (0.03 g, 0.11 mmol). The reaction mixture was stirred at room temperature for 18 hours. Then, water (5 mL), saturated aqueous sodium bicarbonate solution (5 mL) and ethyl acetate (10 mL) were added. After mixing, the precipitate was filtered under vacuum and washed with ethyl acetate. The resulting solid was dried under air to provide 6,7-dimethoxy-2-oxo-1,2-dihydro-quinoline-3-carboxylic acid (2-methyl-5-oxazol-2-yl-phenyl)-amide. (Yield 0.012 g, 37%). HR-MS (ES⁺) m/z Calculated for C₂₂H₂₀N₃O₅ ([M+H]⁺): 406.1398. Found: 406.1397.

### Example 52

### 7-Methoxy-6-(2-methoxy-ethoxy)-2-oxo-1,2-dihydro-quinoline-3-carboxylic acid [5-(3-chloro-benzylcarbamoyl)-2-methyl-phenyl]-amide

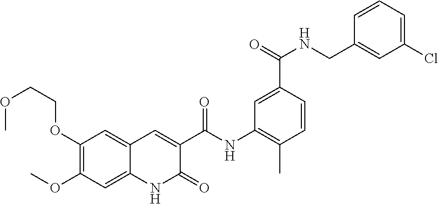

In a 10 mL round-bottomed flask, 3-(7-methoxy-6-(2-methoxyethoxy)-2-oxo-1,2-dihydro-quinoline-3-carboxamido)-4-methylbenzoic acid (0.063 g, 0.148 mmol) (from Example 37 step A supra) was combined with DMF (4 mL) to give a yellow solution. HATU (0.068 g, 0.180 mmol) (Aldrich) and DIPEA (0.023 g, 0.180 mmol) (Aldrich) were added and stirred for 5 mins. 3-Chlorobenzylamine (0.028 mg, 0.196 mmol) (Aldrich) was added and stirred at room temperature overnight. The reaction was poured into water (25 mL), and sat. NaHCO₃ (5 mL) was added, followed by EtOAc (13 mL). After thorough mixing, the mixture was filtered, washed with water (2×5 mL), EtOAc (2×5 mL), dried under reduced pressure for 5 hours to give 7-methoxy-6-(2-methoxy-ethoxy)-2-oxo-1,2-dihydro-quinoline-3-carboxylic acid [5-(3-chloro-benzylcarbamoyl)-2-methyl-phenyl]-amide. (Yield 0.056 g, 69%).

¹H NMR (DMSO-d₆): δ 12.37 (br s, 1H), 9.04 (m, 1H), 8.86 (s, 1H), 8.83 (s, 1H), 7.57-7.54 (m, 2H), 7.41-7.28 (m, 6H), 6.99 (s, 1H), 4.47 (d, 2H), 4.15 (m, 2H), 3.90 (s, 3H), 3.70 (m, 2H), 3.32 (s, 2H), 2.40 (s, 3H). (One NH not detected). HR-MS (ES⁺) m/z Calculated for C₂₉H₂₉ClN₃O₆ ([M+H]⁺): 550.1740. Found: 550.1740.

### Example 53

### Methyl 4-chloro-3-(7-methoxy-6-(2-methoxyethoxy)-2-oxo-1,2-dihydroquinoline-3-carboxamido)benzoate

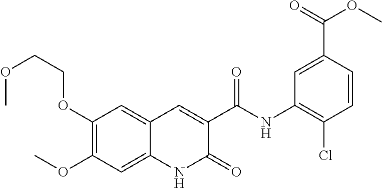

To a solution of 7-methoxy-6-(2-methoxy-ethoxy)-2-oxo-1,2-dihydro-quinoline-3-carboxylic acid (0.23 g, 0.78 mmol) (from Example 23 step D supra) in DMF (10 mL) was added HATU (0.388 g, 1.02 mmol) (Aldrich), triethylamine (0.2 mL) (Aldrich) and methyl-3-amino-4-chloro-benzoate (0.189 g, 1.02 mmol) (TCI). The reaction mixture was stirred at room temperature for 18 hours. Then, water (10 mL), saturated aqueous sodium bicarbonate solution (10 mL) and ethyl acetate (10 mL) were added. After mixing, the precipitate was filtered under vacuum and washed with ethyl acetate. The resulting solid was dried under air to provide methyl 4-chloro-3-(7-methoxy-6-(2-methoxyethoxy)-2-oxo-1,2-dihydroquinoline-3-carboxamido)benzoate. (Yield 0.211 g, 58%).

¹H NMR (DMSO-d₆): δ 12.84 (br s, 1H), 12.44 (br s, 1H), 9.21 (s, 1H), 8.84 (s, 1H), 7.67 (m, 2H), 7.50 (s, 1H), 6.96 (s, 1H), 4.13 (m, 2H), 3.88 (s, 3H), 3.86 (s, 3H), 3.68 (m, 2H), 3.31 (s, 3H). HR-MS (ES⁺) m/z Calculated for C₂₂H₂₂ClN₂O₇ ([M+H]⁺): 461.1110. Found: 461.1110.

### Example 54

### 4-Chloro-3-(7-methoxy-6-(2-methoxyethoxy)-2-oxo-1,2-dihydroquinoline-3-carboxamido)benzoic acid

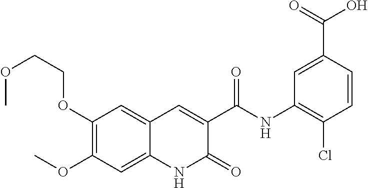

In a 200 mL round-bottomed flask, methyl 4-chloro-3-(7-methoxy-6-(2-methoxyethoxy)-2-oxo-1,2-dihydroquinoline-3-carboxamido)benzoate (0.756 g, 1.64 mmol) (from Example 53 supra) was combined with MeOH (50 mL) to give a light yellow suspension. NaOH (328 mg, 8.2 mmol) and H₂O (5.00 g, 278 mmol) were added and mixture heated at 65° C. for 3 hours. The pH was adjusted to 2. Mixture was filtered, washed with water (20 mL), MeOH (10 mL), dried under reduced pressure overnight to give 4-chloro-3-(7-methoxy-6-(2-methoxyethoxy)-2-oxo-1,2-dihydroquinoline-3-carboxamido)benzoic acid. (Yield 0.698 g, 95%).

¹H NMR (DMSO-d₆): δ 12.81 (br s, 1H), 12.46 (br s, 1H), 9.17 (s, 1H), 8.85 (s, 1H), 7.65 (m, 2H), 7.52 (s, 1H), 6.97 (s, 1H), 4.13 (m, 2H), 3.88 (s, 3H), 3.68 (m, 2H), 3.31 (s, 3H). (One H not detected). HR-MS (ES⁺) m/z Calculated for C₂₁H₂₀ClN₂O₇ ([M+H]⁺): 447.0954. Found: 447.0954.

### Example 55

### N-(2-Chloro-5-(3-chlorobenzylcarbamoyl)phenyl)-7-methoxy-6-(2-methoxyethoxy)-2-oxo-1,2-dihydroquinoline-3-carboxamide

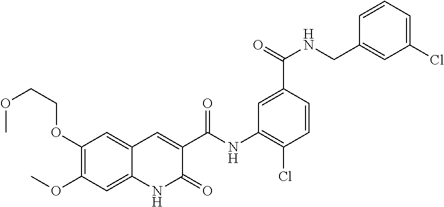

4-Chloro-3-(7-methoxy-6-(2-methoxyethoxy)-2-oxo-1,2-dihydroquinoline-3-carboxamido)-benzoic acid (0.08 g, 0.179 mmol) (from Example 54 supra) was combined with DMF (5 mL) to give a light yellow suspension. HATU (0.0886 g, 0.233 mmol) (Aldrich) and triethylamine (0.1 mL) were added. Then, 3-chlorobenzylamine (0.033 g, 0.233 mmol) (Aldrich) was added. The reaction mixture was stirred for 14 hours. The reaction mixture was diluted with water and a saturated aqueous sodium bicarbonate solution. The reaction mixture was filtered and the yellow solid obtained was dried under air overnight to provide N-(2-chloro-5-(3-chlorobenzylcarbamoyl)phenyl)-7-methoxy-6-(2-methoxyethoxy)-2-oxo-1,2-dihydroquinoline-3-carboxamide. (Yield 0.06 g, 45%).

HR-MS (ES⁺) m/z Calculated for C₂₈H₂₆Cl₂N₃O₆ ([M+H]⁺): 570.1193. Found: 570.1196.

### Example 56

### (R)-N-(5-(2-Hydroxy-1-phenylethylcarbamoyl)-2-methylphenyl)-6,7-dimethoxy-2-oxo-1,2-dihydroquinoline-3-carboxamide

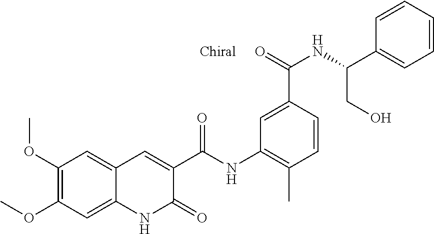

3-(6,7-Dimethoxy-2-oxo-1,2-dihydroquinoline-3-carboxamido)-4-methylbenzoic acid (0.1 g, 0.262 mmol) (from Example 18 supra) was combined with DMF (5 mL) to give a light yellow suspension. HATU (0.129 g, 0.340 mmol) (Aldrich) and triethylamine (0.1 mL) were added. (R)-2-Amino-2-phenylethanol (0.047 g, 0.340 mmol) (Aldrich) was added and the reaction mixture was stirred for 14 hours. The reaction mixture was diluted with water and saturated aqueous sodium bicarbonate solution. The reaction mixture was filtered and the yellow solid obtained was washed with EtOAc followed by methanol and dried under air overnight to provide (R)-N-(5-(2-hydroxy-1-phenylethylcarbamoyl)-2-methylphenyl)-6,7-dimethoxy-2-oxo-1,2-dihydroquinoline-3-carboxamide as a yellow solid. (Yield 0.127 g, 97%). HR-MS (ES⁺) m/z Calculated for C₂₈H₂₈N₃O₆ ([M+H]⁺): 502.1973. Found: 502.1972.

### Example 57

### (S)-N-(5-(2-Hydroxy-1-phenylethylcarbamoyl)-2-methylphenyl)-6,7-dimethoxy-2-oxo-1,2-dihydroquinoline-3-carboxamide

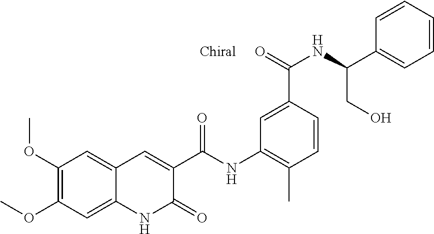

3-(6,7-Dimethoxy-2-oxo-1,2-dihydroquinoline-3-carboxamido)-4-methylbenzoic acid (0.1 g, 0.262 mmol) (from Example 18 supra) was combined with DMF (5 mL) to give a light yellow suspension. HATU (0.129 g, 0.340 mmol) (Aldrich) and triethylamine (0.1 mL) (Aldrich) were added. (S)-2-Amino-2-phenylethanol (0.047 g, 0.340 mmol) (Aldrich) was added and the reaction mixture was stirred for 14 hours. The reaction mixture was diluted with water and saturated aqueous sodium bicarbonate solution. The reaction mixture was filtered and the yellow solid obtained was washed with EtOAc followed by methanol and dried under air overnight to provide (S)-N-(5-(2-hydroxy-1-phenylethylcarbamoyl)-2-methylphenyl)-6,7-dimethoxy-2-oxo-1,2-dihydroquinoline-3-carboxamide as a yellow solid. (Yield 0.105 g, 81%).

HR-MS (ES⁺) m/z Calculated for C₂₈H₂₈N₃O₆ ([M+H]⁺): 502.1973. Found: 502.1973.

### Example 58

### 7-Methoxy-6-(2-methoxy-ethoxy)-2-oxo-1,2-dihydro-quinoline-3-carboxylic acid [5-((R)-3-amino-1-phenyl-propylcarbamoyl)-2-methyl-phenyl]-amide trifluoro-acetic acid salt

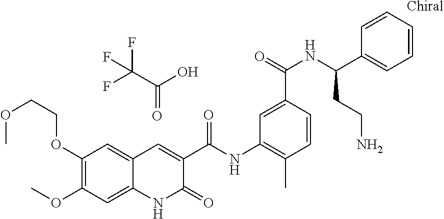

### Step A

### (R)-tert-Butyl 3-(3-(7-methoxy-6-(2-methoxyethoxy)-2-oxo-1,2-dihydroquinoline-3-carboxamido)-4-methylbenzamido)-3-phenylpropylcarbamate

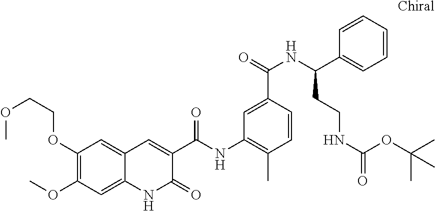

In a 25 mL round-bottomed flask, 3-(7-methoxy-6-(2-methoxyethoxy)-2-oxo-1,2-dihydroquinoline-3-carboxamido)-4-methylbenzoic acid (0.115 g, 0.270 mmol) (from Example 37 step A supra) and HATU (0.123 g, 0.324 mmol) (Aldrich) were combined with DMF (4 mL) to give a light brown suspension. DIPEA (0.042 g, 0.324 mmol) (Aldrich) was added and stirred at room temperature for 5 min to give a clear solution. tert-Butyl 3-amino-3-phenylpropyl-carbamate (0.09 g, 0.359 mmol) (from Example 3 supra) was added and mixture was stirred at room temperature overnight. The reaction mixture was poured into EtOAc (200 mL) and washed with H₂O (2×50 mL) and brine (50 mL). The organic layers were dried over anhydrous sodium sulfate and concentrated under reduced pressure to give an oil.

Chiral separation by supercritical fluid chromatography (Kromasil OD 5-Cellucoat column with 45% MeOH in carbon dioxide) provided the two pure enantiomers as Peak 1 and Peak 2. Peak 1 provided (R)-tert-butyl 3-(3-(7-methoxy-6-(2-methoxyethoxy)-2-oxo-1,2-dihydroquinoline-3-carboxamido)-4-methylbenzamido)-3-phenylpropylcarbamate. (Yield 0.071 g, 80%).

### Step B

### 7-Methoxy-6-(2-methoxy-ethoxy)-2-oxo-1,2-dihydro-quinoline-3-carboxylic acid [5-((R)-3-amino-1-phenyl-propylcarbamoyl)-2-methyl-phenyl]-amide trifluoro-acetic acid salt

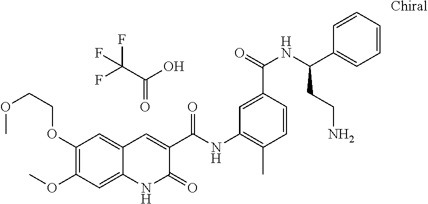

In a 50 mL round-bottomed flask, (R)-tert-butyl 3-(3-(7-methoxy-6-(2-methoxyethoxy)-2-oxo-1,2-dihydroquinoline-3-carboxamido)-4-methylbenzamido)-3-phenylpropylcarbamate (0.071 g, 0.108 mmol) was combined with dichloromethane (10 mL) to give a light yellow solution. TFA (7.4 g, 64.9 mmol) (Aldrich) was added and stirred at room temperature for 2 hours. LC/MS indicated the starting material was consumed. The solvent was removed under reduced pressure. Preparative HPLC provided 7-methoxy-6-(2-methoxy-ethoxy)-2-oxo-1,2-dihydro-quinoline-3-carboxylic acid [5-((R)-3-amino-1-phenyl-propylcarbamoyl)-2-methyl-phenyl]-amide trifluoro-acetic acid salt as a light yellow solid. (Yield 0.053 g, 88%).

HR-MS (ES⁺) m/z Calculated for C₃₁H₃₅N₄O₆ ([M+H]⁺): 559.2551. Found 559.2549.

### Example 59

### 7-Methoxy-6-(2-methoxy-ethoxy)-2-oxo-1,2-dihydro-quinoline-3-carboxylic acid [5-((S)-3-amino-1-phenyl-propylcarbamoyl)-2-methyl-phenyl]-amide trifluoro-acetic acid salt

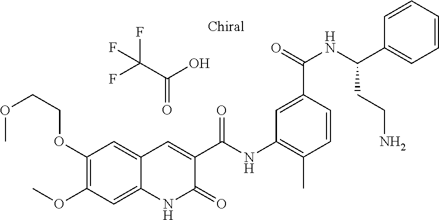

7-Methoxy-6-(2-methoxy-ethoxy)-2-oxo-1,2-dihydro-quinoline-3-carboxylic acid [5-((S)-3-amino-1-phenyl-propylcarbamoyl)-2-methyl-phenyl]-amide trifluoro-acetic acid salt was synthesized in a manner similar to Example 58 with Peak 2 of the chiral separation. (Yield 0.051 g, 81%). HR-MS (ES⁺) m/z Calculated for C₃₁H₃₅N₄O₆ ([M+H]⁺): 559.2551. Found 559.2551.

### Example 60

### N-(5-(2-Amino-1-(3-chlorophenyl)ethylcarbamoyl)-2-chlorophenyl)-7-methoxy-6-(2-methoxyethoxy)-2-oxo-1,2-dihydroquinoline-3-carboxamide

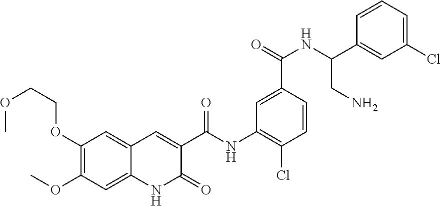

### Step A

### 4-Chloro-3-{[7-methoxy-6-(2-methoxy-ethoxy)-2-oxo-1,2-dihydro-quinoline-3-carbonyl]-amino}-benzoic acid methyl ester

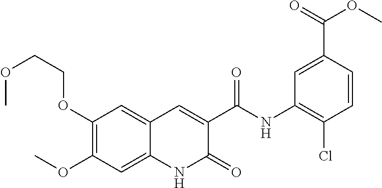

4-Chloro-3-{[7-methoxy-6-(2-methoxy-ethoxy)-2-oxo-1,2-dihydro-quinoline-3-carbonyl]-amino-benzoic acid methyl ester was synthesized in a manner similar to Example 17 with 7-methoxy-6-(2-methoxy-ethoxy)-2-oxo-1,2-dihydro-quinoline-3-carboxylic acid (1 g, 3.41 mmol) (from Example 23 step D supra) and methyl 3-amino-4-chlorobenzoate (0.842 g, 4.54 mmol) (TCI). (Yield 0.756 g, 48%).

¹H NMR (DMSO-d₆): δ 12.84 (br s, 1H), 12.44 (br s, 1H), 9.21 (s, 1H), 8.84 (s, 1H), 7.67 (m, 2H), 7.50 (s, 1H), 6.96 (s, 1H), 4.13 (m, 2H), 3.88 (s, 3H), 3.86 (s, 3H), 3.68 (m, 2H), 3.31 (s, 3H).

### Step B

### 4-Chloro-3-{[7-methoxy-6-(2-methoxy-ethoxy)-2-oxo-1,2-dihydro-quinoline-3-carbonyl]-amino}-benzoic acid

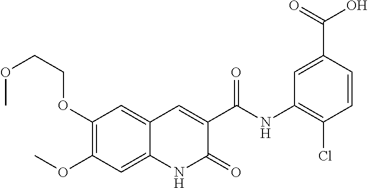

In a 200 mL round-bottomed flask, methyl 4-chloro-3-(7-methoxy-6-(2-methoxyethoxy)-2-oxo-1,2-dihydroquinoline-3-carboxamido)benzoate (0.756 g, 1.64 mmol) was combined with MeOH (50 mL) to give a light yellow suspension. NaOH (0.328 g, 8.2 mmol) and H₂O (5.00 g, 278 mmol) were added and the mixture was heated at 65° C. for 3 hours. The pH was adjusted to 2. Mixture was then filtered, washed with water (20 mL), MeOH (10 mL), dried under reduced pressure overnight to give 4-chloro-3-{[7-methoxy-6-(2-methoxy-ethoxy)-2-oxo-1,2-dihydro-quinoline-3-carbonyl]-amino}-benzoic acid. (Yield 0.698 g, 95%).

¹H NMR (DMSO-d₆): δ 12.81 (br s, 1H), 12.46 (br s, 1H), 9.17 (s, 1H), 8.85 (s, 1H), 7.65 (m, 2H), 7.52 (s, 1H), 6.97 (s, 1H), 4.13 (m, 2H), 3.88 (s, 3H), 3.68 (m, 2H), 3.31 (s, 3H).

### Step C

### N-(5-(2-Amino-1-(3-chlorophenyl)ethylcarbamoyl)-2-chlorophenyl)-7-methoxy-6-(2-methoxyethoxy)-2-oxo-1,2-dihydroquinoline-3-carboxamide

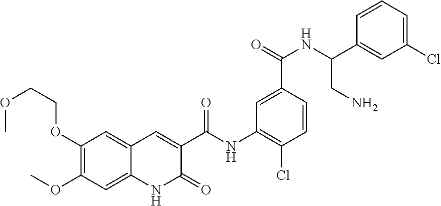

N-(5-(2-Amino-1-(3-chlorophenyl)ethylcarbamoyl)-2-chlorophenyl)-7-methoxy-6-(2-methoxyethoxy)-2-oxo-1,2-dihydroquinoline-3-carboxamide was synthesized in a manner similar to Example 37 step B with 4-chloro-3-{[7-methoxy-6-(2-methoxy-ethoxy)-2-oxo-1,2-dihydro-quinoline-3-carbonyl]-amino}-benzoic acid (0.1 g, 0.224 mmol) and tert-butyl 2-amino-2-(3-chlorophenyl)ethylcarbamate (0.081 g, 0.298 mmol) (from Example 8 supra). (Yield 0.063 g, 74% for two steps).

HR-MS (ES⁺) m/z Calculated for C₂₉H₂₉Cl₂N₄O₆ ([M+H]⁺): 599.1459. Found 599.1460.

### Example 61

### N-(5-(3-Amino-1-phenylpropylcarbamoyl)-2-chlorophenyl)-7-methoxy-6-(2-methoxyethoxy)-2-oxo-1,2-dihydroquinoline-3-carboxamide

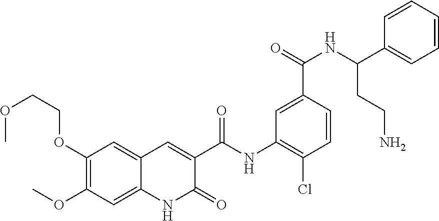

N-(5-(3-Amino-1-phenylpropylcarbamoyl)-2-chlorophenyl)-7-methoxy-6-(2-methoxyethoxy)-2-oxo-1,2-dihydroquinoline-3-carboxamide was synthesized in a manner similar to Example 37 step B with 4-chloro-3-{[7-methoxy-6-(2-methoxy-ethoxy)-2-oxo-1,2-dihydro-quinoline-3-carbonyl]-amino}-benzoic acid (0.1 g, 0.224 mmol) and tert-butyl 3-amino-3-phenylpropylcarbamate (0.149 g, 0.595 mmol) (from Example 3 supra). (Yield 0.063 g, 75% for two steps). HR-MS (ES⁺) m/z Calculated for C₃₀H₃₂ClN₄O₆ ([M+H]⁺): 579.2005. Found 579.2004.

### Example 62

### N-(5-(2-Amino-4-phenylethylcarbamoyl)-2-chlorophenyl)-7-methoxy-6-(2-methoxyethoxy)-2-oxo-1,2-dihydroquinoline-3-carboxamide

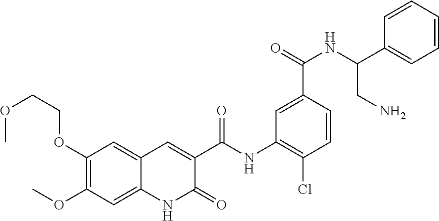

N-(5-(2-Amino-1-phenylethylcarbamoyl)-2-chlorophenyl)-7-methoxy-6-(2-methoxyethoxy)-2-oxo-1,2-dihydroquinoline-3-carboxamide was synthesized in a manner similar to Example 37 step B with 4-chloro-3-{[7-methoxy-6-(2-methoxy-ethoxy)-2-oxo-1,2-dihydro-quinoline-3-carbonyl]-amino}-benzoic acid (0.1 g, 0.224 mmol) and tert-butyl 2-amino-2-phenylethylcarbamate (0.07 g, 0.298 mmol) (from Example 1 supra). (Yield 0.058 g, 85% for two steps). HR-MS (ES⁺) m/z Calculated for C₂₉H₃₀ClN₄O₆ ([M+H]⁺): 565.1849. Found 565.1846.

### Example 63

### (R)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide

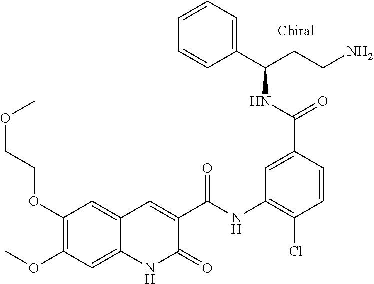

(R)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide was synthesized in a manner similar to Example 58 with 4-chloro-3-{[7-methoxy-6-(2-methoxy-ethoxy)-2-oxo-1,2-dihydro-quinoline-3-carbonyl]-amino}-benzoic acid (0.2 g, 0.448 mmol) with tert-butyl 3-amino-3-phenylpropylcarbamate (0.149 g, 0.595 mmol). (Yield 0.060 g, 46% for two steps). HR-MS (ES⁺) m/z Calculated for C₃₀H₃₂ClN₄O₆ ([M+H]⁺): 579.2005. Found 579.2005.

### Example 64

### (S)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide

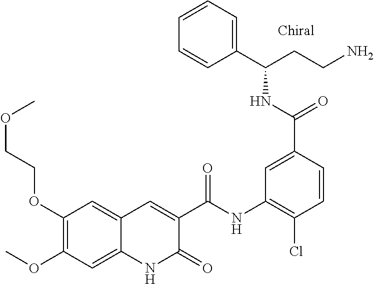

(S)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide was synthesized in a manner similar to Example 58 with 4-chloro-3-{[7-methoxy-6-(2-methoxy-ethoxy)-2-oxo-1,2-dihydro-quinoline-3-carbonyl]-amino}-benzoic acid (0.2 g, 0.448 mmol) with tert-butyl 3-amino-3-phenylpropylcarbamate (0.149 g, 0.595 mmol). (Yield 0.060 g, 43% for two steps). HR-MS (ES⁺) m/z Calculated for C₃₀H₃₂ClN₄O₆ ([M+H]⁺): 579.2005. Found 579.2006.

### Example 65

### (S)-N-(5-(1-Hydroxy-4-methylpentan-2-ylcarbamoyl)-2-methylphenyl)-6,7-dimethoxy-2-oxo-1,2-dihydroquinoline-3-carboxamide

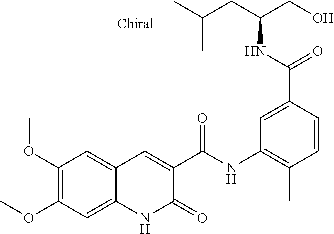

3-(6,7-Dimethoxy-2-oxo-1,2-dihydroquinoline-3-carboxamido)-4-methylbenzoic acid (0.1 g, 0.262 mmol) (from Example 37 step A supra) was combined with DMF (5 mL) to give a light yellow suspension. HATU (0.129 g, 0.340 mmol) (Aldrich) and triethylamine (0.1 mL) were added. (S)-2-Amino-4-methylpentan-1-ol (L-leucinol) (0.04 g, 0.340 mmol) (Aldrich) was added and the reaction mixture was stirred for 14 hours. The reaction mixture was diluted with water and saturated aqueous sodium bicarbonate solution. The reaction mixture was filtered and the yellow solid obtained was washed with EtOAc and dried under air overnight to provide (S)-N-(5-(1-hydroxy-4-methylpentan-2-ylcarbamoyl)-2-methylphenyl)-6,7-dimethoxy-2-oxo-1,2-dihydroquinoline-3-carboxamide as a yellow solid. (Yield 0.082 g, 65%). HR-MS (ES⁺) m/z Calculated for C₂₆H₃₂N₃O₆ ([M+H]⁺): 482.2286. Found: 482.2287.

### Example 66

### (R)-N-(5-(1-Hydroxy-4-methylpentan-2-ylcarbamoyl)-2-methylphenyl)-6,7-dimethoxy-2-oxo-1,2-dihydroquinoline-3-carboxamide

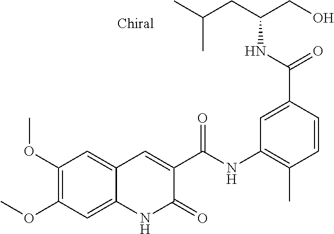

3-(6,7-Dimethoxy-2-oxo-1,2-dihydroquinoline-3-carboxamido)-4-methylbenzoic acid (0.1 g, 0.262 mmol) (from Example 37 step A supra) was combined with DMF (5 mL) to give a light yellow suspension. HATU (0.129 g, 0.340 mmol) (Aldrich) and triethylamine (0.1 mL) (Aldrich) were added. (R)-2-Amino-4-methylpentan-1-ol (D-leucinol) (0.04 g, 0.340 mmol) (Aldrich) was added and the reaction mixture was stirred for 14 hours. The reaction mixture was diluted with water and saturated aqueous sodium bicarbonate solution. The reaction mixture was filtered and the yellow solid obtained was washed with EtOAc and dried under air overnight to provide (R)-N-(5-(1-hydroxy-4-methylpentan-2-ylcarbamoyl)-2-methylphenyl)-6,7-dimethoxy-2-oxo-1,2-dihydroquinoline-3-carboxamide as a yellow solid. (Yield 0.03 g, 22%). HR-MS (ES⁺) m/z Calculated for C₂₆H₃₂N₃O₆ ([M+H]⁺): 482.2286. Found: 482.2286.

### Example 67

### 6-Benzyloxy-7-methoxy-2-oxo-1,2-dihydro-quinoline-3-carboxylic acid [5-(3-chloro-benzylcarbamoyl)-2-methyl-phenyl]-amide

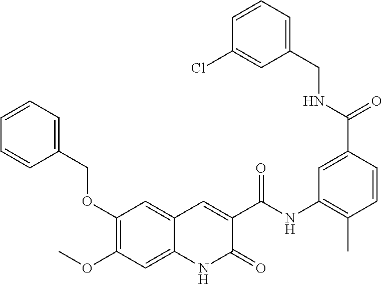

### Step A

### 3-Benzyloxy-4-methoxy-benzaldehyde

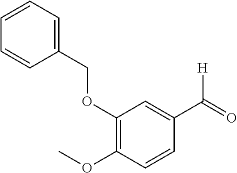

To a stirred solution 3-hydroxy-4-methoxy-benzaldehyde (20.0 g, 131 mmol) (Aldrich) in ethanol (300 mL) was added potassium carbonate (12.89 g, 93.40 mmol) at room temperature. After 30 minutes of stirring at the same temperature, benzyl chloride (32.0 mL, 2.76 mmol) was added into the reaction mixture and the resulting solution was heated at reflux at 100° C. for 5 hours (monitored by silica TLC; mobile phase: ethyl acetate-hexanes 3:7). After cooling to room temperature, reaction mixture was concentrated under reduced pressure and the obtained crude material was diluted with ethyl acetate (600 mL). The organic layer was washed with an aqueous saturated solution of potassium carbonate (200 mL), dried over anhydrous Na₂SO₄, filtered and was concentrated under reduced pressure to obtain a crude mixture, which was purified over silica gel (100-200 mesh) column chromatography, eluted with ethyl acetate-hexanes (1:19), to give 3-benzyloxy-4-methoxy-benzaldehyde as white solid. (Yield 28.0 g, 87.93%).

### Step B

### 5-Benzyloxy-4-methoxy-2-nitro-benzaldehyde

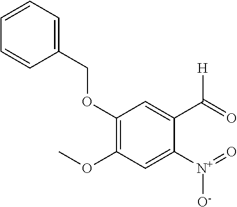

To a stirred solution of 3-benzyloxy-4-methoxy-benzaldehyde (25 g, 103.30 mmol) in dichloromethane (250 mL) was added dilute nitric acid (125 mL) at 0° C. After 30 minutes of stirring at the same temperature, another portion of dilute nitric acid (125 mL) was added at room temperature and the resulting reaction mixture was stirred for another 30 minutes (monitored by silica TLC; mobile phase: ethyl acetate-hexanes 1:4). Reaction mixture was then diluted with ice-water (250 mL) and was extracted with dichloromethane (2×250 mL). Collected organic parts were dried over anhydrous sodium sulfate and concentrated under reduced pressure to give crude 5-benzyloxy-4-methoxy-2-nitro-benzaldehyde as yellow solid, which was pure enough to proceed for the next step without further purification. (Yield 65 g, 73%).

### Step C

### 2-Amino-5-benzyloxy-4-methoxy-benzaldehyde

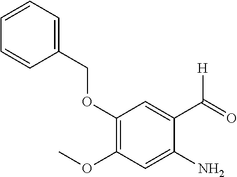

To a stirred solution of 5-benzyloxy-4-methoxy-2-nitro-benzaldehyde (15 g, 52.26 mmol) in a mixture of ethanol-acetic acid-water (2:2:1; 200 mL) was added iron (23.35 g, 418.18 mmol) and hydrochloric acid (10 mL) at room temperature and the resulting reaction mixture was heated at reflux at 100° C. for 30 minutes (monitored by silica TLC; mobile phase: ethyl acetate-hexanes 3:7). After cooling to room temperature, the reaction mixture was filtered through a Celite bed and the filtrate was concentrated under reduced pressure to give crude 2-amino-5-benzyloxy-4-methoxy-benzaldehyde as a black solid, which was use for next step reaction without further purification. (Yield 35 g, 65%).

### Step D

### 6-Benzyloxy-7-methoxy-2-oxo-1,2-dihydro-quinoline-3-carboxylic acid methyl ester

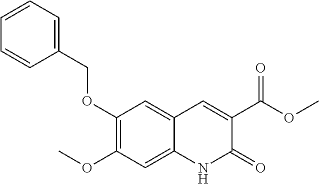

To a stirred solution of 2-amino-5-benzyloxy-4-methoxy-benzaldehyde (1.0 g, 3.89 mmol) in methanol (20 mL) were added dimethyl malonate (0.89 mL, 7.78 mmol) (Aldrich), piperidine (0.77 mL, 7.78 mmol) (Aldrich) and acetic acid (0.1 mL) sequentially at 0° C. and the resulting reaction mixture was slowly heated at reflux at 100° C. for 12 hours (monitored by silica TLC; mobile phase: methanol-dichloromethane, 1:9). After cooling to room temperature, the reaction mixture was concentrated under reduced pressure and the obtained crude material was purified over silica gel (100-200 mesh) column chromatography, using methanol-dichloromethane, 1:19 as eluent, to give 6-benzyloxy-7-methoxy-2-oxo-1,2-dihydro-quinoline-3-carboxylic acid methyl ester as a brown solid. (Yield 0.156 g, 11.7%).

### Step E

### 6-Benzyloxy-7-methoxy-2-oxo-1,2-dihydro-quinoline-3-carboxylic acid

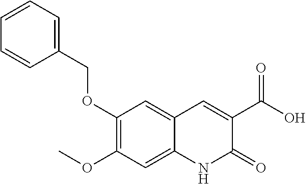

To a stirred solution of 6-benzyloxy-7-methoxy-2-oxo-1,2-dihydro-quinoline-3-carboxylic acid methyl ester (0.350 g, 1.03 mmol) in tetrahydrofuran (10 mL) was added a solution of lithium hydroxide (0.216 g, 5.16 mmol) (Aldrich) in water (2.5 mL) and the reaction mixture was stirred for 12 hours at room temperature (monitored by silica TLC; methanol-dichloromethane, 1:9). Solvents were distilled off under reduced pressure; the obtained lithiated salt was acidified with citric acid [1% (w/v)] solution at 0° C. and was extracted with (2×20 mL). Collected organic parts were dried over anhydrous sodium sulfate, filtered, and concentrated under reduced pressure to give 6-benzyloxy-7-methoxy-2-oxo-1,2-dihydro-quinoline-3-carboxylic acid as brown solid. (Yield 0.220 g, 65.8%).

### Step F

### 3-tert-Butoxycarbonylamino-4-methyl-benzoic acid methyl ester

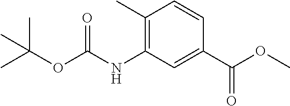

To a stirred solution of 3-amino-4-methyl-benzoic acid methyl ester (1.00 g, 6.06 mmol) (Aldrich) in tetrahydrofuran (20 mL) was added boc anhydride (1.4 mL, 6.42 mmol) and the resulting reaction mixture was heated at reflux for 36 hours. (The reaction was monitored by silica TLC; ethyl acetate-hexanes, 3:7). Tetrahydrofuran was distilled off under reduced pressure to give crude 3-tert-butoxycarbonylamino-4-methyl-benzoic acid methyl ester as white solid. (Yield 1.20 g, 74.7%).

### Step G

### 3-tert-Butoxycarbonylamino-4-methyl-benzoic acid

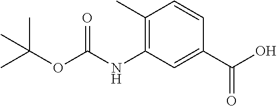

To a stirred solution of 3-tert-butoxycarbonylamino-4-methyl-benzoic acid methyl ester (0.35 g, 1.32 mmol) in 1,4-dioxane (10 mL) was added an aqueous solution of sodium hydroxide (1N, 2.5 mL) and reaction mixture was stirred at room temperature for 12 hours (monitored by silica TLC; ethyl acetate-hexanes, 1:1). Water was distilled off under reduced pressure; obtained crude sodium salt was acidified with acetic acid solution at 0° C. and was extracted with (2×20 mL). Collected organic parts were dried over anhydrous sodium sulfate, filtered, and concentrated under reduced pressure to give 3-tert-butoxycarbonylamino-4-methyl-benzoic acid as off white solid, which was taken to the next step without further purification. (Yield 1.20 g, 74.7%).

### Step H

### [5-(3-Chloro-benzylcarbamoyl)-2-methyl-phenyl]carbamic acid tert-butyl ester

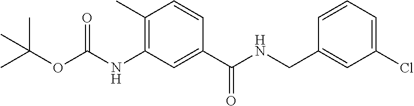

To a stirred solution of 3-tert-butoxycarbonylamino-4-methyl-benzoic acid (0.160 g, 0.64 mmol) in dichloromethane (5 mL) were added 1-hydroxybenzotriazole (0.172 g, 1.27 mmol) (Aldrich), N-(3-dimethylaminopropyl)-N′-ethylcarbodiimide hydrochloride (0.244 g, 1.27 mmol) (Aldrich), triethyl amine (0.17 mL, 1.91 mmol) (Aldrich) and 3-chloro-benzylamine (0.07 mL, 1.00 mmol) (Aldrich) sequentially at room temperature under nitrogen and the resulting mixture was stirred for 1 hour (monitored by silica TLC; ethyl acetate-hexanes, 1:1). Solvent was distilled off under reduced pressure; obtained crude material was diluted with ice-water (10 mL) and was extracted with dichloromethane (3×25 mL). Collected organic parts were dried over anhydrous sodium sulfate, filtered, and concentrated under reduced pressure to give crude [5-(3-chloro-benzyl-carbamoyl)-2-methyl-phenyl]carbamic acid tert-butyl ester as off white solid, which was used for the next step reaction without further purification. (Yield 0.223 g, 93%).

### Step I

### 3-Amino-N-(3-chloro-benzyl)-4-methyl-benzamide

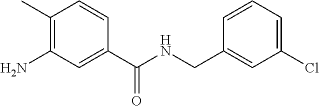

To a stirred solution of [5-(3-chloro-benzyl-carbamoyl)-2-methyl-phenyl]carbamic acid tert-butyl ester (0.220 g, 0.59 mmol) in dichloromethane (10 mL) was added trifluoroacetic acid (0.65 mL, 8.8 mmol) at 0° C. and the resulting solution was stirred for 8 hours at room temperature (monitored by silica TLC; ethyl acetate-hexanes, 1:1). Dichloromethane was distilled off under reduced pressure, obtained residue was diluted with water (15 mL), basified with ammonia solution (to pH about 8) and was extracted with ethyl acetate (3×20 mL). Collected organic parts were dried over anhydrous sodium sulfate, filtered, and concentrated under reduced pressure to obtain a crude material, which was purified over silica gel (100-200 mesh) column chromatography, eluted with ethyl acetate-hexanes, 1:1, to give 3-amino-N-(3-chloro-benzyl)-4-methyl-benzamide as off white solid. (Yield 0.110 g, 68.5%).

### Step J

### 6-Benzyloxy-7-methoxy-2-oxo-1,2-dihydro-quinoline-3-carboxylic acid [5-(3-chloro-benzylcarbamoyl)-2-methyl-phenyl]-amide

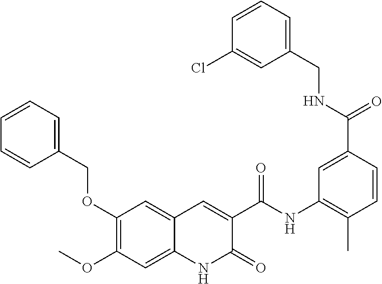

To a stirred solution of 6-benzyloxy-7-methoxy-2-oxo-1,2-dihydro-quinoline-3-carboxylic acid (0.400 g, 1.2 mmol) in THF (20 mL) was added HATU (1.4 g, 3.69 mmol) at room temperature under nitrogen. After 30 minutes of stirring at the same temperature, N-methyl morpholine (0.51 mL, 3.69 mmol) was added drop-wise into the reaction mixture followed by the addition of 3-amino-N-(3-chloro-benzyl)-4-methyl-benzamide (0.438 g, 1.59 mmol) and the resulting solution was heated at reflux at 80° C. for 12 hours (monitored by silica TLC; ethyl acetate-hexanes, 9:1). After cooling to room temperature, reaction mixture was concentrated under reduced pressure, obtained crude residue was diluted with ice water (20 mL) and was extracted with dichloromethane (3×25 mL). Collected organic parts were dried over anhydrous sodium sulfate, filtered, and concentrated under reduced pressure to obtain a crude material, which was purified over silica gel (100-200 mesh) column chromatography, eluted with ethyl acetate-hexanes, 4:1, to give 6-benzyloxy-7-methoxy-2-oxo-1,2-dihydro-quinoline-3-carboxylic acid [5-(3-chloro-benzylcarbamoyl)-2-methyl-phenyl]-amide as off white solid. (Yield 0.150 g, 21.0%). LR-MS [M+H]⁺582.

### Example 68

### 6-Hydroxy-7-methoxy-2-oxo-1,2-dihydro-quinoline-3-carboxylic acid [5-(3-chloro-benzylcarbamoyl)-2-methyl-phenyl]-amide

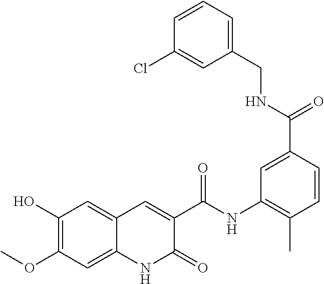

To a stirred solution of 6-benzyloxy-7-methoxy-2-oxo-1,2-dihydro-quinoline-3-carboxylic acid [5-(3-chloro-benzylcarbamoyl)-2-methyl-phenyl]amide (0.160 g, 0.27 mmol) (from Example 67 supra) in dichloromethane (5 mL) was added boron trichloride (0.03 mL, 0.33 mmol) drop-wise at 0° C. and the resulting solution was stirred for 4 hours at the same temperature (monitored by silica TLC; ethyl acetate-hexanes, 9:1). Reaction was quenched with methanol-water mixture (1:1; 2 mL) and was concentrated under reduced pressure. Obtained crude material was diluted with ice-water (15 mL) and was extracted with dichloromethane (3×25 mL). Collected organic extracts were dried over anhydrous sodium sulfate, filtered, and concentrated under reduced pressure to obtain a crude material, which was purified over silica gel (100-200 mesh) column chromatography, eluted with ethyl acetate-hexanes, 4:1, to give 6-hydroxy-7-methoxy-2-oxo-1,2-dihydro-quinoline-3-carboxylic acid [5-(3-chloro-benzylcarbamoyl)-2-methyl-phenyl]-amide as light yellow solid. (Yield 0.060 g, 44.5%). LR-MS [M+H]⁺492.

### Example 69

### 6-Benzyloxy-7-methoxy-2-oxo-1,2-dihydro-quinoline-3-carboxylic acid [2-chloro-5-(3-chloro-benzylcarbamoyl)-phenyl]-amide

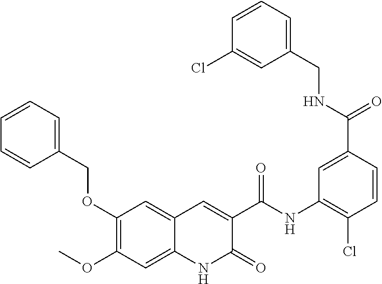

### Step A

### 3-Amino-4-chloro-benzoic acid

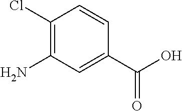

To a stirred solution of 3-amino-4-chloro-benzoic acid methyl ester (0.300 g, 1.62 mmol) in tetrahydrofuran (20 mL) was added a solution of lithium hydroxide (0.340 g, 8.10 mmol) in water (5 mL) and reaction was stirred at room temperature for 12 hours (monitored by silica TLC; ethyl acetate-hexanes, 1:1). Solvent was distilled off under reduced pressure; the obtained lithium salt was acidified with citric acid [1% (w/v)] solution at 0° C. and was extracted (2×20 mL). Collected organic parts were dried over anhydrous sodium sulfate, filtered and concentrated under reduced pressure to give 3-amino-4-chloro-benzoic acid as white solid. (Yield 0.200 g, 72.01%).

### Step B

### 3-Amino-4-chloro-N-(3-chloro-benzyl)-benzamide

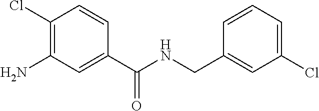

To a stirred solution of 3-amino-4-chloro-benzoic acid (0.200 g, 1.16 mmol) in DMF (10 mL) was added HBTU (0.487 g, 1.27 mmol), DIPEA (0.58 mL, 3.50 mmol) and 3-chlorobenzylamine (0.14 mL, 1.00 mmol) and the resulting mixture was stirred for 4 hours at room temperature under nitrogen (monitored by silica TLC; ethyl acetate-hexanes, 1:1). Solvent was distilled off under reduced pressure, obtained crude material was diluted with ice-water (10 mL) and was extracted with dichloromethane (3×25 mL). Collected organic parts were dried over anhydrous sodium sulfate, filtered and concentrated under reduced pressure to give 3-amino-4-chloro-N-(3-chloro-benzyl)-benzamide as off white solid, which was used in the next step reaction without further purification. (Yield 0.300 g, 72%).

### Step C

### 6-Benzyloxy-7-methoxy-2-oxo-1,2-dihydro-quinoline-3-carboxylic acid [2-chloro-5-(3-chloro-benzylcarbamoyl)-phenyl]-amide

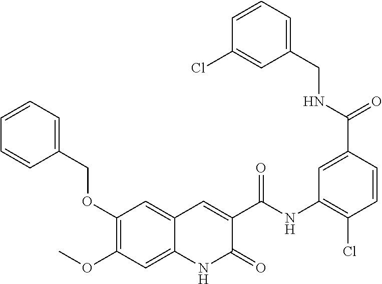

To a stirred solution of 6-benzyloxy-7-methoxy-2-oxo-1,2-dihydro-quinoline-3-carboxylic acid (0.500 g, 1.5 mmol) (from Example 67 Step E supra) in tetrahydrofuran (10 mL) was added HATU (1.75 g, 4.61 mmol) at room temperature under nitrogen. After 30 minutes of stirring at the same temperature, N-methyl morpholine (0.51 mL, 4.61 mmol) was added drop-wise followed by 3-amino-4-chloro-N-(3-chloro-benzyl)-benzamide (0.635 g, 2.15 mmol) and the resulting reaction mixture was heated at reflux at 80° C. for 12 hours (monitored by silica TLC; ethyl acetate-hexanes, 4:1). After cooling to room temperature, reaction mixture was concentrated under reduced pressure, obtained crude residue was diluted with ice-water (20 mL) and was extracted with dichloromethane (3×25 mL). Collected organic parts were dried over anhydrous sodium sulfate, filtered, and concentrated under reduced pressure to obtain a crude material, which was purified over silica gel (100-200 mesh) column chromatography, eluted with ethyl acetate-hexanes, 4:1, to give 6-benzyloxy-7-methoxy-2-oxo-1,2-dihydro-quinoline-3-carboxylic acid [2-chloro-5-(3-chloro-benzylcarbamoyl)-phenyl]amide as light yellow solid. (Yield 0.200 g, 21.5%). LR-MS [M+H]⁺602.

### Example 70

### 7-Methoxy-6-[2-(2-methoxy-ethoxy)-ethoxy]-2-oxo-1,2-dihydro-quinoline-3-carboxylic acid [5-(3-chloro-benzylcarbamoyl)-2-methyl-phenyl]-amide

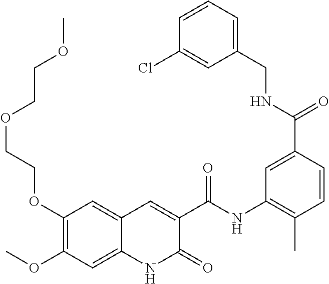

### Step A

### Toluene-4-sulfonic acid 2-(2-methoxy-ethoxy)-ethyl ester

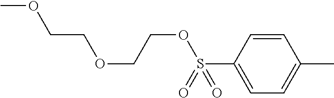

To a stirred solution of 2-(2-methoxy-ethoxy)-ethanol (2.0 g, 16.64 mmol) in diethyl ether (20 mL) was added sodium hydroxide (2.0 g, 49.93 mmol) at room temperature. After 10 minutes of stirring at the same temperature, p-tosyl chloride (3.16 g, 16.64 mmol) was added and reaction mixture was stirred for 5 hours at room temperature (monitored by silica TLC; ethyl acetate-hexanes, 1:1). Reaction mass was diluted with diethyl ether (50 mL), washed with water (20 mL), dried over anhydrous sodium sulfate, filtered, and concentrated under reduced pressure to give toluene-4-sulfonic acid 2-(2-methoxy-ethoxy)-ethyl ester as colorless liquid. (Yield 3.0 g, 66%).

### Step B

### 5-Hydroxy-4-methoxy-2-nitro-benzaldehyde

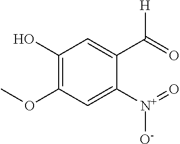

5-Benzyloxy-4-methoxy-2-nitro-benzaldehyde (2.00 g, 6.97 mmol) (from Example 67 Step B supra) was mixed with trifluoroacetic acid (10 mL) at 0° C. and the resulting solution was stirred at 60° C. for 16 hours (monitored by silica TLC; ethyl acetate-hexanes, 3:7). TFA was distilled off under reduced pressure and the obtained residue was triturated with diethyl ether and dry under reduced pressure to give crude 5-hydroxy-4-methoxy-2-nitro-benzaldehyde as brown solid, which was used for the next step reaction without further purification. (Yield 1.2 g, 87%).

### Step C

### 4-Methoxy-5-[2-(2-methoxy-ethoxy)-ethoxy]-2-nitro-benzaldehyde

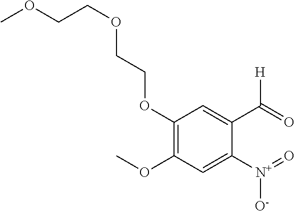

Cesium carbonate (6.90 g, 21.31 mmol) was added to a stirred solution of 5-hydroxy-4-methoxy-2-nitro-benzaldehyde (1.40 g, 7.10 mmol) in DMF (20 mL) at room temperature under nitrogen. After 30 minutes of stirring at the same temperature, toluene-4-sulfonic acid 2-(2-methoxy-ethoxy)-ethyl ester (5.86 g, 21.31 mmol) was added at room temperature and the resulting reaction mixture was stirred at 60° C. for 16 hours (monitored by silica TLC experiment; mobile phase: ethyl acetate-hexanes, 1:4). DMF was distilled off under reduced pressure and the crude material was diluted with ice-water (20 mL) and was extracted with dichloromethane (2×25 mL). Collected organic parts were dried over anhydrous sodium sulfate, filtered and concentrated under reduced pressure to give crude 4-methoxy-5-[2-(2-methoxy-ethoxy)-ethoxy]-2-nitro-benzaldehyde as black solid, which was pure enough to proceed for the next step without further purification. (Yield 1.6, 75.3%).

### Step D

### 2-Amino-4-methoxy-5-[2-(2-methoxy-ethoxy)-ethoxy]-benzaldehyde

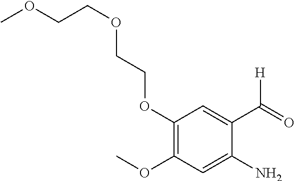

To a stirred solution of 4-methoxy-5-[2-(2-methoxy-ethoxy)-ethoxy]-2-nitro-benzaldehyde (1.60 g, 5.35 mmol) in a mixture of ethanol-acetic acid-water (2:2:1; 10 mL) was added iron (2.39 g, 42.80 mmol) at room temperature and the resulting reaction mixture was heated at reflux at 100° C. for 30 minutes (monitored by silica TLC; mobile phase: ethyl acetate-hexanes, 2:3). After cooling to room temperature, the reaction mixture was filtered through a Celite bed and the filtrate was concentrated under reduced pressure to give crude 2-amino-4-methoxy-5-[2-(2-methoxy-ethoxy)-ethoxy]-benzaldehyde as black solid, which was use for next step reaction without further purification. (Yield 1.4 g, 97.2%).

### Step E

### 7-Methoxy-6-[2-(2-methoxy-ethoxy)-ethoxy]-2-oxo-1,2-dihydro-quinoline-3-carboxylic acid methyl ester

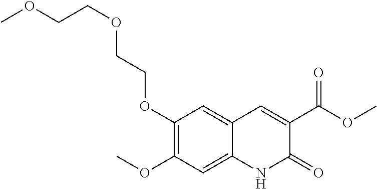

To a stirred solution of 2-amino-4-methoxy-5-[2-(2-methoxy-ethoxy)-ethoxy]-benzaldehyde (1.4 g, 5.20 mmol) in methanol (20 mL) was added dimethyl malonate (1.37 g, 10.40 mmol), piperidine (1.0 mL, 10.40 mmol) and acetic acid (0.3 mL) sequentially at 0° C. and the resulting reaction mixture was slowly heated at reflux at 100° C. for 12 hours (monitored by silica TLC; mobile phase: methanol-dichloromethane, 1:9). After cooling to room temperature, the reaction mixture was concentrated under reduced pressure to obtain 7-methoxy-6-[2-(2-methoxy-ethoxy)-ethoxy]-2-oxo-1,2-dihydro-quinoline-3-carboxylic acid methyl ester as black solid, which was use for next step reaction without further purification. (Yield 0.09 g, 5%).

### Step F

### 7-Methoxy-6-[2-(2-methoxy-ethoxy)-ethoxy]-2-oxo-1,2-dihydro-quinoline-3-carboxylic acid

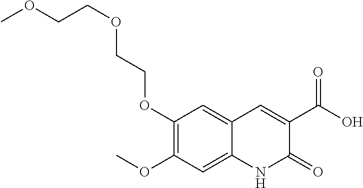

To a stirred solution of 7-methoxy-6-[2-(2-methoxy-ethoxy)-ethoxy]-2-oxo-1,2-dihydro-quinoline-3-carboxylic acid methyl ester (0.090 g, 0.25 mmol) in tetrahydrofuran (5 mL) was added a solution of lithium hydroxide (0.032 g, 0.77 mmol) in water (1.2 mL) and reaction mixture was stirred for 12 hours at room temperature (monitored by silica TLC; methanol-dichloromethane, 1:9). Solvents were distilled off under reduced pressure, the obtained lithium salt was acidified with citric acid [1% (w/v)] solution at 0° C. and was extracted (2×20 mL). Collected organic parts were dried over anhydrous sodium sulfate, filtered and concentrated under reduced pressure to give 7-methoxy-6-[2-(2-methoxy-ethoxy)-ethoxy]-2-oxo-1,2-dihydro-quinoline-3-carboxylic acid as brown solid. (Yield 0.05 g, 57.9%).

### Step G

### 7-Methoxy-6-[2-(2-methoxy-ethoxy)-ethoxy]-2-oxo-1,2-dihydro-quinoline-3-carboxylic acid [5-(3-chloro-benzylcarbamoyl)-2-methyl-phenyl]-amide

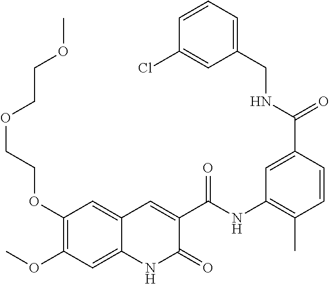

To a stirred solution of 7-methoxy-6-[2-(2-methoxy-ethoxy)-ethoxy]-2-oxo-1,2-dihydro-quinoline-3-carboxylic acid (0.05 g, 0.15 mmol) in tetrahydrofuran (5 mL) was added HATU (0.170 g, 0.44 mmol) at room temperature under nitrogen. After 30 minutes of stirring at the same temperature, N-methyl morpholine (0.06 mL, 0.44 mmol) was added drop-wise into the reaction mixture followed by the addition of 3-amino-N-(3-chloro-benzyl)-4-methyl-benzamide (0.050 g, 0.20 mmol) (from Example 67 Step I supra) and the resulting solution was heated at refluxed at 80° C. for 12 hours (monitored by silica TLC; ethyl acetate-hexanes, 9:1). After cooling to room temperature, reaction mixture was concentrated under reduced pressure, obtained crude residue was diluted with ice water (20 mL) and was extracted with dichloromethane (3×25 mL). Collected organic parts were dried over anhydrous sodium sulfate, filtered, and concentrated under reduced pressure to obtain a crude material, which was purified over silica gel (100-200 mesh) column chromatography, eluted with ethyl acetate-hexanes, 4:1, to give 7-methoxy-6-[2-(2-methoxy-ethoxy)-ethoxy]-2-oxo-1,2-dihydro-quinoline-3-carboxylic acid [5-(3-chloro-benzylcarbamoyl)-2-methyl-phenyl]amide as light yellow solid. (Yield 0.012 g, 13.4%). LR-MS [M+H]⁺594.

### Example 71

### 4-Chloro-3-[(6,7-dimethoxy-2-oxo-1,2,3,4-tetrahydro-quinoline-3-carbonyl)-amino]-benzoic acid methyl ester

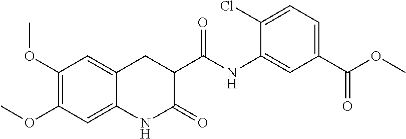

### Step A

### Diethyl 2-(4,5-dimethoxy-2-nitrobenzylidene)malonate

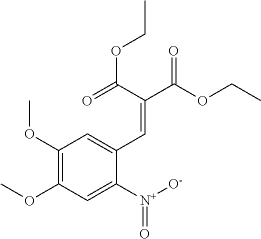

In a 100 mL pear-shaped flask, 4,5-dimethoxy-2-nitro-benzaldehyde (4.02 g, 19.0 mmol, 1.00 eq) (Aldrich) was suspended in acetic anhydride (10.0 mL) (Aldrich). Diethyl malonate (4.22 g, 4.0 mL, 26.3 mmol, 1.38 eq) (Aldrich) and sodium bicarbonate (3.35 g, 39.9 mmol, 2.09 eq) were added and the mixture was heated at 100° C. and stirred for 6 hours. After cooling to room temperature, the reaction mixture was partitioned between water and EtOAc. The organic phase was removed, washed with saturated sodium bicarbonate and brine, dried over Na₂SO₄ and concentrated. The crude material was purified by flash chromatography (Analogix; SF65-300, Si50; 30-100% EtOAc in hexanes). The product-containing fractions were combined, concentrated and re-crystallized to give diethyl 2-(4,5-dimethoxy-2-nitrobenzylidene)malonate. (Yield 3.476 g, 9.84 mmol, 51.7%).

### Step B

### Diethyl 2-(4,5-dimethoxy-2-nitrobenzyl)malonate

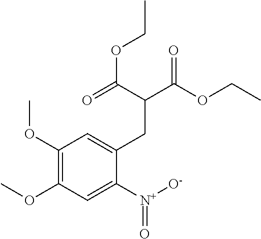

In a 25 mL pear-shaped flask, diethyl 2-(4,5-dimethoxy-2-nitrobenzylidene)malonate (707 mg, 2 mmol, 1.00 eq) was suspended in ethanol (5 mL). Sodium cyanoborohydride (137 mg. 2.18 mmol, 1.09 eq) (Aldrich) was added. Bromocresol indicator was added and then concentrated hydrochloric acid was added as needed to maintain the yellow color. After stirring overnight, the reaction was judged to be complete and the reaction was concentrated to remove the ethanol. The aqueous residue was extracted with ethyl acetate, dried over sodium sulfate and concentrated to give crude diethyl 2-(4,5-dimethoxy-2-nitrobenzyl)malonate. (Yield 0.746 g; 2.1 mmol, 105%).

### Step C

### Ethyl 6,7-dimethoxy-2-oxo-1,2,3,4-tetrahydroquinoline-3-carboxylate

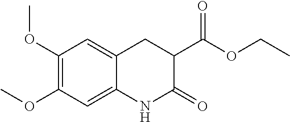

Diethyl 2-(4,5-dimethoxy-2-nitrobenzyl)malonate (0.385 g; 0.975 mmol, 1.00 eq) was combined with acetic acid (40 mL) and heated in an oil bath to 90-100° C. Iron (0.500 g, 8.95 mmol, 9.18 eq) was added portionwise to the hot mixture over 30 minutes. Stirring continued for another 60 minutes and then the hot, dark red mixture was filtered through a bed of Celite. The filtrate was concentrated and resulting residue neutralized with saturated sodium bicarbonate. The solid was collected and washed with water. The crude material was purified by flash chromatography (Analogix, SF25-60 g Si; dichloromethane containing 2-3% methanol) to give ethyl 6,7-dimethoxy-2-oxo-1,2,3,4-tetrahydroquinoline-3-carboxylate. (Yield 0.223 g, 0.77 mmol, 78%).

### Step D

### 6,7-Dimethoxy-2-oxo-1,2,3,4-tetrahydroquinoline-3-carboxylic acid

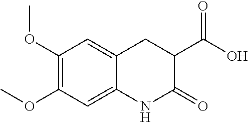

In a 25 mL pear-shaped flask, ethyl 6,7-dimethoxy-2-oxo-1,2,3,4-tetrahydroquinoline-3-carboxylate (211 mg, 755 mmol, 1.00 eq) was suspended in tetrahydrofuran, (1.6 mL), methanol (0.40 mL) and water (1.6 mL). Lithium hydroxide (36.2 mg, 1.51 mmol, 2.00 eq) was added. A clear solution resulted which was stirred for 90 minutes at room temperature. The solution was concentrated and the residue was dissolved in water and acidified. The solid which precipitated out of solution was collected by filtration, washed with water followed by ether and then dried to give 6,7-dimethoxy-2-oxo-1,2,3,4-tetrahydroquinoline-3-carboxylic acid. (Yield 188.7 mg, 0.751 mmol, 99.4%).

### Step E

### 4-Chloro-3-[(6,7-dimethoxy-2-oxo-1,2,3,4-tetrahydro-quinoline-3-carbonyl)-amino]-benzoic acid methyl ester

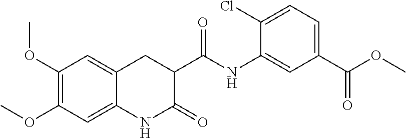

In a 10 mL pear-shaped flask, 6,7-dimethoxy-2-oxo-1,2,3,4-tetrahydroquinoline-3-carboxylic acid (91.0 mg, 0.362 mmol, 1.00 eq) and methyl 3-amino-4-chlorobenzoate (101 mg, 0.543 mmol, 1.50 eq) (TCI) were combined in DMF (1.1 mL). Triethylamine (0.25 mL, 1.79 mmol, 4.95 eq) and then HATU (207 mg, 0.543 mmol, 1.50 eq) (Aldrich) were added resulting in a yellow solution. After stirring for 90 minutes at room temperature the reaction was complete and was diluted with EtOAc. When no precipitation of the product occurred, the organic solution was extracted twice with water and once with brine and then concentrated. The solid residue was triturated first with water and then with hot ethyl acetate to give 4-chloro-3-[(6,7-dimethoxy-2-oxo-1,2,3,4-tetrahydro-quinoline-3-carbonyl)-amino]-benzoic acid methyl ester. (Yield 73.2 mg, 0.171 mmol, 47.3%).

HR-MS (ES⁺) m/z Calculated for C₂₀H₁₉ClN₂NaO₆ ([M+Na]⁺): 441.0824. Found: 441.0825

### Example 72

### 3-[(6,7-Dimethoxy-2-oxo-1,2,3,4-tetrahydro-quinoline-3-carbonyl)-amino]-4-methyl-benzoic acid methyl ester

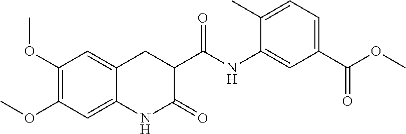

In a 10 mL pear-shaped flask, 6,7-dimethoxy-2-oxo-1,2,3,4-tetrahydroquinoline-3-carboxylic acid (92.0 mg, 0.366 mmol, 1.00 eq) (from Example 71, Step D supra) and methyl 3-amino-4-methylbenzoate (90.7 mg, 0.549 mmol, 1.50 eq) (TCI) were combined in DMF (1.1 mL). Triethylamine (0.255 mL, 1.83 mmol, 5.00 eq) and then HATU (209 mg, 0.540 mmol, 1.50 eq) (Aldrich) were added resulting in a yellow solution. After stirring for 3 hours at room temperature the reaction was diluted with EtOAc, extracted twice with water and once with brine and then concentrated. The solid residue was triturated with water and then with hot EtOAc to give 3-[(6,7-dimethoxy-2-oxo-1,2,3,4-tetrahydro-quinoline-3-carbonyl)-amino]-4-methyl-benzoic acid methyl ester. (Yield 97.2 mg, 0.244 mmol, 66.6%).

HR-MS (ES⁺) m/z Calculated for C₂₁H₂₂N₂NaO₆ ([M+Na]⁺): 421.1370. Found: 421.1371.

### Example 73

### 6,7-Dimethoxy-2-oxo-1,2,3,4-tetrahydro-quinoline-3-carboxylic acid (5-benzylcarbamoyl-2-chloro-phenyl)-amide

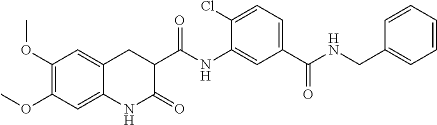

### Step A

### 4-Chloro-3-(6,7-dimethoxy-2-oxo-1,2,3,4-tetrahydroquinoline-3-carboxamido)benzoic acid

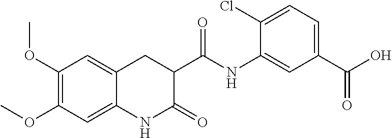

4-Chloro-3-[(6,7-dimethoxy-2-oxo-1,2,3,4-tetrahydro-quinoline-3-carbonyl)-amino]-benzoic acid methyl ester (0.652 g, 1.56 mmol, 1.00 eq) (from Example 71 supra) was suspended in tetrahydrofuran (8 mL), water (2 mL) and methanol (8 mL). Lithium hydroxide (76.7 mg, 3.2 mmol, 2.05 eq) was added and additional methanol and DMSO were added to dissolve all of the material. After stirring for 72 hours the reaction was concentrated. The residue was taken up in water and acidified with 6N HCl. The yellow solid was collected by filtration, washed with water and dried under house vacuum giving crude 4-chloro-3-(6,7-dimethoxy-2-oxo-1,2,3,4-tetrahydroquinoline-3-carboxamido)benzoic acid. (Yield 0.657 g). The material was very impure but was used in the next step without further purification.

### Step B

### N-(5-(Benzylcarbamoyl)-2-chlorophenyl)-6,7-dimethoxy-2-oxo-1,2,3,4-tetrahydroquinoline-3-carboxamide

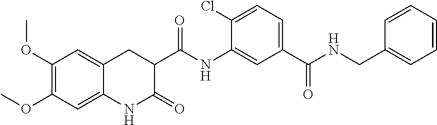

In a 10 mL pear-shaped flask, crude 4-chloro-3-(6,7-dimethoxy-2-oxo-1,2,3,4-tetrahydro-quinoline-3-carboxamido)benzoic acid (93.4 mg, 231 μmol, eq: 1.00) was suspended in DMF (1 mL). Triethylamine (116 mg, 1.15 mmol, eq: 4.98) was added followed by HATU (123 mg, 323 μmol, eq: 1.40) (Aldrich). Material initially went into solution and then a yellow solid precipitated out. Benzylamine (34.3 mg, 320 μmol, eq: 1.39) (Aldrich)) was added. Solid went back into solution. After stirring at room temperature for 4 hours, the reaction was complete but complex because of the impurities that were carried through from the previous step. The crude material was purified by reverse phase chromatography in multiple runs (Gilson and ISCO systems). The pure fractions were combined and concentrated. The residue was then triturated with water. The solid was collected by filtration, washed with water and dried to give N-(5-(benzylcarbamoyl)-2-chlorophenyl)-6,7-dimethoxy-2-oxo-1,2,3,4-tetrahydroquinoline-3-carboxamide. (Yield 12.3 mg, 24.9 μmol, 10.8%). HR-MS (ES⁺) m/z Calculated for C₂₆H₂₅ClN₃O₅ ([M+H]⁺): 494.1477. Found: 494.1477.

### Example 74

### 6,7-Dimethoxy-2-oxo-1,2,3,4-tetrahydro-quinoline-3-carboxylic acid (5-benzylcarbamoyl-2-methyl-phenyl)-amide

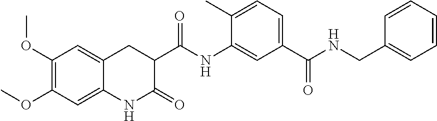

In a 10 mL pear-shaped flask, 6,7-dimethoxy-2-oxo-1,2,3,4-tetrahydroquinoline-3-carboxylic acid (58.7 mg, 234 μmol, eq: 1.00) (from Example 71 step D supra) was combined with DMF (9 mL) to give a colorless solution. HATU (124 mg, 327 μmol, eq: 1.40) (Aldrich) was added, followed by triethylamine (94.6 mg, 935 μmol, eq: 4.00) and finally 3-amino-N-benzyl-4-methylbenzamide (78.6 mg, 327 μmol, eq: 1.40) (from Example 13 supra) dissolved in DMF (0.3 mL). After stirring overnight at room temperature the reaction was still incomplete. Additional HATU (94 mg) was added and stirring continued at room temperature for additional 24 hours. The crude reaction mixture was purified by reverse phase HPLC (Gilson; multiple injections) without any workup. The pure fractions from each run were combined and concentrated to a small volume (˜2 mL). The solid which had precipitated out of solution was collected by filtration and washed with water. The solid was then suspended in water/acetonitrile and freeze-dried to give 6,7-dimethoxy-2-oxo-1,2,3,4-tetrahydro-quinoline-3-carboxylic acid (5-benzylcarbamoyl-2-methyl-phenyl)-amide. (Yield 29.5 mg, 62.3 μmol, 26.7%). HR-MS (ES⁺) m/z Calculated for C₂₇H₂₈N₃O₅ ([M+H]⁺): 474.2024. Found: 474.2023.

The pharmacological properties of the compounds of this invention may be confirmed by a number of pharmacological assays. The exemplified enzyme and antiproliferative activity assays which follow have been carried out with the compounds according to the invention.

If test compounds were assessed in multiple runs of the same assay, the activities reported in Table I are the averages of the results obtained from the multiple runs of the assay.

### Example 75

### DYRK1B Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement (or % inhibition at 10 μM), is a measure of a test compound's ability to interfere with the phosphorylation of the peptide substrate, that is it inhibits the phosphorylation of the substrate peptide by DYRK1B, and is thus a measure of the test compound's ability to inhibit the activity of DYRK1B. IC₅₀ is the amount of test compound that inhibits 50% of the activity of DYRK1B in this assay. In some cases where the IC₅₀ was not determined, then the % inhibition at 10 μM test compound concentration may be reported instead. The results of this assay for sample compounds of the invention are provided in Table I below.

### Materials and Reagents

1. Human DYRK1B: from Invitrogen. Part # PR8350B (former PV4649)

### 2. Substrate Peptide: RRRFRPASPLRGPPK

3. IMAP TR-FRET IPP Explorer Kit: from Molecular Devices. Part # R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat#23490-102
6. Detection Plate: Costa black 384-well microplate. Cat #3710
Assay Procedure: This assay was performed as follows:
1. Transfer 1.5 μL of 20× compound solution to each well of an assay plate.
2. Add to each well 22.5 μL of KAB Buffer.
3. Add to each well 3 μL of the solution of DYRK1B and ATP. The final concentration of DYRK1B is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.

### 10. Calculation:

TR-FRET Signal=(Reading at 520 nM/Reading at 490 nM)×2000000

### Example 76

### DYRK1A Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement, is a measure of a test compound's ability to interfere with the phosphorylation of the peptide substrate, that is it inhibits the phosphorylation of the substrate peptide by DYRK1A, and is thus a measure of the test compound's ability to inhibit the activity of DYRK1A. IC₅₀ is the amount of test compound that inhibits 50% of the activity of DYRK1A in this assay. In some cases where the IC₅₀ was not determined, then the % inhibition at 10 μM test compound concentration may be reported instead. The results of this assay for sample compounds of the invention are provided in Table I below.

### Materials and Reagents

1. Human DYRK1A: from Invitrogen. Part # PV3997

### 2. Substrate Peptide: RRRFRPASPLRGPPK

3. IMAP TR-FRET IPP Explorer Kit: from Molecular Devices. Part # R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat#23490-102
6. Detection Plate: Costa black 384-well microplate. Cat #3710
Assay Procedure: This assay was performed as follows:
1. Transfer 1.5 μL of 20× compound solution to each well of an assay plate.
2. Add to each well 22.5 μL of KAB Buffer.
3. Add to each well 3 μL of the solution of DYRK1A and ATP. The final concentration of DYRK1A is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.

### 10. Calculation:

TR-FRET Signal=(Reading at 520 nM/Reading at 490 nM)×2000000

### Example 77

### SW620 Cell Viability Assay

1. Cell Plate Preparation: SW620 human colon cancer cells (known to express DYRK1B), obtained from ATCC, were seeded into 96-well plates at 3×10³ cells/well in 50 μL of media.
Harvested the required number of cells (counts & viability determined by Guava Viacount).
Centrifuge cells to pellet and removed supernant.
Resuspended in growth media (50 μL/well) & pipetted thoroughly to break up clumps.
Setup for ˜100 wells/plate, therefore, V_{T}=5 mL/plate @ 1.2×10⁵ cells/mL.
   1A. Some cells (S-) were allowed to attach for 24 hrs, serum starved for 48 hrs, and then followed with treatments of test compound.

Test Compound solution was prepared in regular corresponding media supplemented with serum

2. Test Compound Preparation: Test compounds were solubilized in either DMSO or media and prepared at various stock concentrations.
All compounds were incubated at 37° C. for 30 minutes and vortexed.
   (5 mM stocks of test compound were prepared for non-soluble drugs)

### 3. Test Compound Plate Preparation:

The 10 mM test compound stock was diluted to a concentration of 100× the final Cₘₐₓ concentration.
Then the test compound stock is diluted 50-fold in media and/or second test compound for a final Cₘₐₓ concentration in the test compound plate.
The Cₘₐₓ in the test compound plate (2% DMSO) is 2-fold higher than the final Cₘₐₓ in the cell plate (60 uL titrations were 1:3).
   4. Viability Assay: This assay was performed as follows:
Transfer 50 μL of test compound solution per well from the test compound plate onto the cell plate prepared in Step #1.
Mix the plate with treated cells by pipetting up and down three times with 200 μL multi-channel pipet.
Incubate the cells in 5% CO₂ incubator @ 37° C. for 4 days.
Run the CellTiter-Glo™ Luminescent Cell Viability Assay.
   The results of this assay, given as EC₅₀ values, indicate the concentration of test compound that inhibits tumor cell proliferation by 50%. The results of this assay for sample compounds of the invention are provided in Table I below.

TABLE 1
Kinase enzyme and cellular activity

| | Enzyme | Enzyme | Cellular |
|---|---|---|---|
| | IC₅₀ | IC₅₀ | EC₅₀ |
| | (μM)* | (μM)* | (μM) |
| Example | DYRK1B | DYRK1A | SW620 |
| 17 | 0.56 | 0.32 | >10 |
| 19 | 47% | 44% | >10 |
| 20 | 5.085 | 1.128 | |
| 21 | 5.477 | >10 | |
| 22 | 0.719 | >10 | |
| 23 | 0.44 | 0.262 | >10 |
| 24 | 2.666 | 1.762 | |
| 25 | 0.911 | 0.604 | |
| 26 | 6.421 | 5.136 | |
| 27 | 6.588 | 41% | |
| 28 | 5.045 | 49% | |
| 31 | 0.141 | 0.0598 | 1.92 |
| 32 | >10 | 3.625 | |
| 33 | 36% | 36% | |
| 35 | 0.0595 | 0.154 | 2.05 |
| 36 | 28% | 0.239 | |
| 37 | 0.0325 | 0.0191 | 1.82 |
| 38 | 0.0448 | 0.0347 | 2.76 |
| 39 | 0.19 | 0.15 | 3.41 |
| 40 | 34% | 0.182 | |
| 41 | 2.593 | 4.585 | |
| 42 | 0.32 | 0.226 | >10 |
| 43 | 0.178 | 0.006 | 0.306 |
| 44 | 0.063 | 0.0334 | >3.0 |
| 47 | 0.014 | 0.0855 | >3.0 |
| 48 | 39% | 1.016 | |
| 50 | 0.0732 | 0.008 | 1.13 |
| 51 | 42% | 0.592 | |
| 52 | 0.181 | 0.118 | 0.78 |
| 53 | 0.115 | 0.245 | >10 |
| 55 | 0.0373 | 0.0489 | 0.129 |
| 56 | >10 | 31% | |
| 57 | 0.883 | 0.186 | 6.5 |
| 58 | 0.029 | 0.009 | 4.04 |
| 59 | 4.91 | 2.38 | |
| 60 | 0.0217 | 0.0143 | 0.2 |
| 61 | 0.0115 | 0.0157 | 0.47 |
| 62 | 0.0291 | 0.0114 | 0.44 |
| 63 | 0.008 | 0.005 | 0.64 |
| 64 | 0.253 | 0.186 | 9.2 |
| 65 | 26% | 4.31 | |
| 66 | 13% | >10 | |
| 68 | 0.189 | 0.058 | |
| 70 | 1.69 | 0.067 | |
| 71 | 0.86 | 0.69 | |
| 72 | 1.8 | 5.59 | |
| 73 | 2.34 | 0.88 | |
| 74 | >10 | 30% | |

*% measurements reflect % inhibition at 10 μM test compound concentration.

What is claimed:

1. A compound of formula I 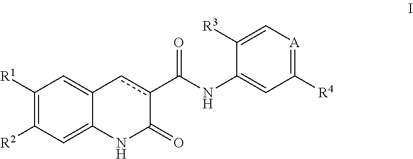
wherein [symbol]
means the presence of a bond (that is, a double bond is present in the above structure) or no bond (meaning a single bond is present);
A is CH, CF or N;
R¹ is selected from the group consisting of
(a) OH,
(b) OR⁵, and
(c) NR⁶R⁷;
R² is selected from the group consisting of
(a) OR⁵, and
(b) NR⁶R⁷;
R³ is selected from the group consisting of
(a) C₁₋₄ alkyl,
(b) F,
(c) Cl, and
(d) Br;
R⁴ is selected from the group consisting of
(a) COR^{S},
(b) Tetrazol-5-yl, and
(c) Oxazol-2-yl,
and when A is N, R⁴ can also be H;
R⁵ is selected from the group consisting of
(a) C₁₋₄ alkyl,
(b) C₁₋₄ alkyl substituted with up to 3 groups selected from
heterocycle,
OH,
OC₁₋₄ alkyl, wherein the alkyl optionally may be substituted with one or more alkoxy groups,
NR⁹R¹⁰, and
CN;
R⁶ and R⁷ are each independently selected from the group consisting of
(a) H,
(b) C₁₋₄ alkyl, and
(c) C₂₋₄ alkyl substituted with up to 3 groups selected from
heterocycle,
OH,
OC₁₋₄ alkyl,
NR⁹R¹⁰, and
CN;
R⁸ is selected from the group consisting of
(a) OR¹¹, and
(b) NR¹²R¹³;
R⁹ and R¹⁹ are independently selected from the group consisting of
(a) H, and
(b) C₁₋₄ alkyl;
R¹¹ is selected from the group consisting of
(a) C₁₋₆ alkyl,
(b) C₁₋₆ alkyl substituted with up to 3 groups selected from
aryl,
aryl optionally substituted with Cl, F, CH₃,
heteroaryl,
cycloalkyl,
heterocycle,
OH,
OC₁₋₄ alkyl,
NR⁹R¹⁰,
CN, and
CONR⁹R¹⁰, and
(c) aryl optionally substituted with Cl, F, CH₃;
R¹² and R¹³ are independently selected from the group consisting of
(a) H,
(b) C₁₋₆ alkyl,
(c) C₁₋₆ alkyl substituted with up to 3 groups selected from
aryl,
aryl optionally substituted with Cl, F, CH₃,
heteroaryl,
cycloalkyl,
heterocycle,
OH,
OC₁₋₄ alkyl,
NR⁹R¹⁰,
CN,
CONR⁹R¹⁰, and
(d) Aryl optionally substituted with Cl, F, CH₃,
or, alternately, NR¹²R¹³ together can form a heterocycle, optionally substituted with
(a) Cl,
(b) F,
(c) CH₃,
(d) aryl optionally substituted with Cl, F, CH₃, and
(e) heteroaryl optionally substituted with Cl, F, CH₃;
or a pharmaceutically acceptable salt thereof.

2. A compound of claim 1, wherein—is a bond, having the formula 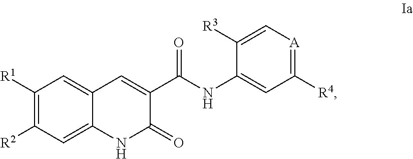
or a pharmaceutically acceptable salt thereof.

3. A compound of claim 1, wherein—is not a bond, having the formula 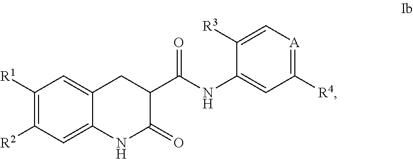
or a pharmaceutically acceptable salt thereof.

4. The compound of claim 1, wherein A is CH, or a pharmaceutically acceptable salt thereof.

5. The compound of claim 1, wherein A is N, or a pharmaceutically acceptable salt thereof.

6. The compound of claim 1, wherein R¹ is OR⁵, and R⁵ is C₁₋₄ alkyl or C₁₋₄ alkyl substituted with heterocycle, OC₁₋₄alkyl or NR⁹R¹⁰, or a pharmaceutically acceptable salt thereof.

7. The compound of claim 6, wherein R¹ is OR⁵ and R⁵ selected from CH₃, CH₂CH₂OCH₃ and CH₂CH₂-heterocycle, or a pharmaceutically acceptable salt thereof.

8. The compound of claim 7, wherein R² is OR⁵ and R⁵ is C₁₋₄ alkyl, or a pharmaceutically acceptable salt thereof.

9. The compound of claim 8, wherein R² is OCH₃, or a pharmaceutically acceptable salt thereof.

10. The compound of claim 8, wherein R³ is C₁₋₄ alkyl or Cl, or a pharmaceutically acceptable salt thereof.

11. The compound of claim 10, wherein R³ is CH₃, or a pharmaceutically acceptable salt thereof.

12. The compound of claim 10, wherein R⁴ is COR⁸, and R⁸ is OR¹¹, or a pharmaceutically acceptable salt thereof.

13. The compound of claim 10, wherein R⁴ is COR⁸, and R⁸ is OR¹¹ and R¹¹ is C₁₋₆ alkyl, or a pharmaceutically acceptable salt thereof.

14. The compound of claim 13, wherein R¹¹ is CH₃, or a pharmaceutically acceptable salt thereof.

15. The compound of claim 12, wherein R⁴ is COR⁸ and R⁸ is NR¹²R¹³.

16. The compound of claim 15, wherein R⁴ is COR⁸, R⁸ is NR¹²R¹³ and R¹² and R¹³ are independently selected from H and C₁₋₆ alkyl, or a pharmaceutically acceptable salt thereof.

17. The compound of claim 16, wherein R¹² and R¹³ are each CH₃, or a pharmaceutically acceptable salt thereof.

18. The compound of claim 15, wherein R⁴ is COR⁸, R⁸ is NR¹²R¹³ and R¹² and R¹³ are independently selected from H, C₁₋₆ alkyl, and C₁₋₆ alkyl that is substituted with up to 3 groups selected from aryl, heteroaryl, OH, C₁₋₄ alkyl, heterocycle, cycloalkyl, and NR⁹R¹⁰, or a pharmaceutically acceptable salt thereof.

19. The compound of claim 18, wherein the group NR⁹R¹⁰ is NH₂.

20. The compound of claim 1, wherein A is N and R⁴ is H.

21. The compound of claim 1, wherein R⁴ is tetrazol-5-yl or oxazol-2-yl.

22. A compound of formula 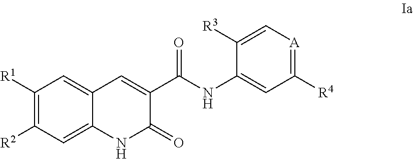
wherein
A is CH or N;
R¹ is selected from the group consisting of OH, OCH₃, OCH₂CH₂OCH₃, OCH₂CH₂-4-morpholinyl and O(CH₂)₂O(CH₂)₂OCH₃;
R² is selected from the group consisting of OCH₃, OCH₂CH₂OCH₃;
R³ is selected from the group consisting of CH₃ and Cl;
R⁴ is selected from the group consisting of C(O)OCH₃, C(O)N(CH3)₂, tetrazol-5-yl and COR⁸;
R⁸ is NR¹²R¹³; and
R¹² and R¹³ are each independently selected from H, and
C₁₋₆ alkyl substituted with NH₂, OH, morpholin-4-yl, cyclohexyl and phenyl optionally substituted with Cl;
or a pharmaceutically acceptable salt thereof.

23. A compound of formula Ib 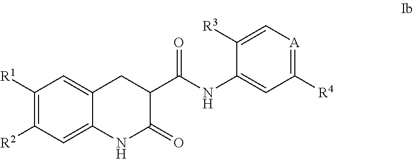
wherein
A is CH;
R¹ and R² are OCH₃;
R³ is C₁ or CH₃; and
R⁴ is C(O)OCH₃ or C(O)NHCH₂-phenyl;
or a pharmaceutically acceptable salt thereof.

24. A compound selected from the group consisting of
3-[(6,7-Dimethoxy-2-oxo-1,2-dihydro-quinoline-3-carbonyl)-amino]-4-methyl-benzoic acid methyl ester;
4-Chloro-3-[(6,7-dimethoxy-2-oxo-1,2-dihydro-quinoline-3-carbonyl)-amino]-benzoic acid methyl ester;
6,7-Dimethoxy-2-oxo-1,2-dihydro-quinoline-3-carboxylic acid (2-chloro-5-dimethylcarbamoyl-phenyl)-amide;
6,7-Dimethoxy-2-oxo-1,2-dihydro-quinoline-3-carboxylic acid (3-methyl-pyridin-4-yl)-amide;
6,7-Dimethoxy-2-oxo-1,2-dihydro-quinoline-3-carboxylic acid [2-methyl-5-(1H-tetrazol-5-yl)-phenyl]-amide;
3-{[7-Methoxy-6-(2-methoxy-ethoxy)-2-oxo-1,2-dihydro-quinoline-3-carbonyl]-amino}-4-methyl-benzoic acid methyl ester;
7-Methoxy-6-(2-methoxy-ethoxy)-2-oxo-1,2-dihydro-quinoline-3-carboxylic acid [2-methyl-5-(1H-tetrazol-5-yl)-phenyl]-amide;
3-{[6,7-Bis-(2-methoxy-ethoxy)-2-oxo-1,2-dihydro-quinoline-3-carbonyl]-amino}-4-methyl-benzoic acid methyl ester;
3-{[6-Methoxy-7-(2-methoxy-ethoxy)-2-oxo-1,2-dihydro-quinoline-3-carbonyl]-amino}-4-methyl-benzoic acid methyl ester; and
6-Methoxy-7-(2-methoxy-ethoxy)-2-oxo-1,2-dihydro-quinoline-3-carboxylic acid [2-methyl-5-(1H-tetrazol-5-yl)-phenyl]-amide;
or a pharmaceutically acceptable salt of any of the foregoing compounds.

25. A compound selected from the group consisting of
6,7-Bis-(2-methoxy-ethoxy)-2-oxo-1,2-dihydro-quinoline-3-carboxylic acid [2-methyl-5-(1-tetrazol-5-yl)-phenyl]-amide;
6,7-Dimethoxy-2-oxo-1,2-dihydro-quinoline-3-carboxylic acid [5-(2-amino-1-phenyl-ethylcarbamoyl)-2-methyl-phenyl]-amide;
6,7-Dimethoxy-2-oxo-1,2-dihydro-quinoline-3-carboxylic acid [2-methyl-5-(3-morpholin-4-yl-propylcarbamoyl)-phenyl]-amide;
6,7-Dimethoxy-2-oxo-1,2-dihydro-quinoline-3-carboxylic acid [5-(1-hydroxymethyl-2-methyl-propylcarbamoyl)-2-methyl-phenyl]-amide;
6,7-Dimethoxy-2-oxo-1,2-dihydro-quinoline-3-carboxylic acid [5-(3-amino-1-phenyl-propylcarbamoyl)-2-methyl-phenyl]-amide;
6,7-Dimethoxy-2-oxo-1,2-dihydro-quinoline-3-carboxylic acid (3-chloro-pyridin-4-yl)-amide;
7-Methoxy-6-(2-methoxy-ethoxy)-2-oxo-1,2-dihydro-quinoline-3-carboxylic acid [5-(2-amino-1-phenyl-ethylcarbamoyl)-2-methyl-phenyl]-amide;
7-Methoxy-6-(2-methoxy-ethoxy)-2-oxo-1,2-dihydro-quinoline-3-carboxylic acid [5-(3-amino-1-phenyl-propylcarbamoyl)-2-methyl-phenyl]amide;
3-{[7-Methoxy-6-(2-morpholin-4-yl-ethoxy)-2-oxo-1,2-dihydro-quinoline-3-carbonyl]-amino}-4-methyl-benzoic acid methyl ester; and
6,7-Dimethoxy-2-oxo-1,2-dihydro-quinoline-3-carboxylic acid [5-(3-chloro-benzylcarbamoyl)-2-methyl-phenyl]-amide;
or a pharmaceutically acceptable salt of any of the foregoing compounds.

26. A compound from the selected group consisting of
6,7-Dimethoxy-2-oxo-1,2-dihydro-quinoline-3-carboxylic acid [5-(cyclohexylmethyl-carbamoyl)-2-methyl-phenyl]-amide;
6,7-Dimethoxy-2-oxo-1,2-dihydro-quinoline-3-carboxylic acid (5-benzylcarbamoyl-2-methyl-phenyl)-amide;
7-Methoxy-6-(2-morpholin-4-yl-ethoxy)-2-oxo-1,2-dihydro-quinoline-3-carboxylic acid [5-(3-chloro-benzylcarbamoyl)-2-methyl-phenyl]-amide;
7-Methoxy-6-(2-morpholin-4-yl-ethoxy)-2-oxo-1,2-dihydro-quinoline-3-carboxylic acid [5-(3-amino-1-phenyl-propylcarbamoyl)-2-methyl-phenyl]-amide;
6,7-Dimethoxy-2-oxo-1,2-dihydro-quinoline-3-carboxylic acid [5-((R)-3-amino-1-phenyl-propylcarbamoyl)-2-methyl-phenyl]-amide;
6,7-Dimethoxy-2-oxo-1,2-dihydro-quinoline-3-carboxylic acid [5-((S)-3-amino-1-phenyl-propylcarbamoyl)-2-methyl-phenyl]-amide;
6,7-Dimethoxy-2-oxo-1,2-dihydro-quinoline-3-carboxylic acid {5-[2-amino-1-(3-chloro-phenyl)-ethylcarbamoyl]-2-methyl-phenyl}-amide;
6,7-Dimethoxy-2-oxo-1,2-dihydro-quinoline-3-carboxylic acid (2-methyl-5-oxazol-2-yl-phenyl)-amide;
7-Methoxy-6-(2-methoxy-ethoxy)-2-oxo-1,2-dihydro-quinoline-3-carboxylic acid [5-(3-chloro-benzylcarbamoyl)-2-methyl-phenyl]-amide; and
Methyl 4-chloro-3-(7-methoxy-6-(2-methoxyethoxy)-2-oxo-1,2-dihydroquinoline-3-carboxamido)benzoate;
or a pharmaceutically acceptable salt of any of the foregoing compounds.

27. A compound selected from the group consisting of
N-(2-Chloro-5-(3-chlorobenzylcarbamoyl)phenyl)-7-methoxy-6-(2-methoxyethoxy)-2-oxo-1,2-dihydroquinoline-3-carboxamide;
(R)-N-(5-(2-Hydroxy-1-phenylethylcarbamoyl)-2-methylphenyl)-6,7-dimethoxy-2-oxo-1,2-dihydroquinoline-3-carboxamide;
(S)-N-(5-(2-Hydroxy-1-phenylethylcarbamoyl)-2-methylphenyl)-6,7-dimethoxy-2-oxo-1,2-dihydroquinoline-3-carboxamide;
7-Methoxy-6-(2-methoxy-ethoxy)-2-oxo-1,2-dihydro-quinoline-3-carboxylic acid [5-((R)-3-amino-1-phenyl-propylcarbamoyl)-2-methyl-phenyl]-amide trifluoro-acetic acid salt;
7-Methoxy-6-(2-methoxy-ethoxy)-2-oxo-1,2-dihydro-quinoline-3-carboxylic acid [5-((S)-3-amino-1-phenyl-propylcarbamoyl)-2-methyl-phenyl]-amide trifluoro-acetic acid salt;
N-(5-(2-Amino-1-(3-chlorophenyl)ethylcarbamoyl)-2-chlorophenyl)-7-methoxy-6-(2-methoxyethoxy)-2-oxo-1,2-dihydroquinoline-3-carboxamide;
N-(5-(3-Amino-1-phenylpropylcarbamoyl)-2-chlorophenyl)-7-methoxy-6-(2-methoxyethoxy)-2-oxo-1,2-dihydroquinoline-3-carboxamide;
N-(5-(2-Amino-1-phenylethylcarbamoyl)-2-chlorophenyl)-7-methoxy-6-(2-methoxyethoxy)-2-oxo-1,2-dihydroquinoline-3-carboxamide;
(R)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide;
(S)-N-(5-(3-Amino-1-phenylpropylcarbamoyl)-2-chlorophenyl)-7-methoxy-6-(2-methoxyethoxy)-2-oxo-1,2-dihydroquinoline-3-carboxamide;
(S)-N-(5-(1-Hydroxy-4-methylpentan-2-ylcarbamoyl)-2-methylphenyl)-6,7-dimethoxy-2-oxo-1,2-dihydroquinoline-3-carboxamide;
(R)-N-(5-(1-Hydroxy-4-methylpentan-2-ylcarbamoyl)-2-methylphenyl)-6,7-dimethoxy-2-oxo-1,2-dihydroquinoline-3-carboxamide;
6-Hydroxy-7-methoxy-2-oxo-1,2-dihydro-quinoline-3-carboxylic acid [5-(3-chloro-benzylcarbamoyl)-2-methyl-phenyl]-amide; and
7-Methoxy-6-[2-(2-methoxy-ethoxy)-ethoxy]-2-oxo-1,2-dihydro-quinoline-3-carboxylic acid [5-(3-chloro-benzylcarbamoyl)-2-methyl-phenyl]-amide;
or a pharmaceutically acceptable salt of any of the foregoing compounds.

28. A compound selected from the group consisting of
4-Chloro-3-[(6,7-dimethoxy-2-oxo-1,2,3,4-tetrahydro-quinoline-3-carbonyl)-amino]-benzoic acid methyl ester;
3-[(6,7-Dimethoxy-2-oxo-1,2,3,4-tetrahydro-quinoline-3-carbonyl)-amino]-4-methyl-benzoic acid methyl ester;
6,7-Dimethoxy-2-oxo-1,2,3,4-tetrahydro-quinoline-3-carboxylic acid (5-benzylcarbamoyl-2-chloro-phenyl)-amide; and
6,7-Dimethoxy-2-oxo-1,2,3,4-tetrahydro-quinoline-3-carboxylic acid (5-benzylcarbamoyl-2-methyl-phenyl)-amide;
or a pharmaceutically acceptable salt of any of the foregoing compounds.

29. A pharmaceutical composition comprising a compound of claim 1, or a pharmaceutically acceptable salt thereof, as an active ingredient and a pharmaceutically acceptable carrier or excipient.
